SEQUENCE LISTING (56 sequences)

SEQ ID NO: 1 - DNA, 2628 bp, Neisseria meningitidis (group B)
atgcccgccggccgactgccccgccgatgcccgatgatgacgaaatttacagactgtacgcggtcaaaccgtattcagccgccaacccacaggggatacatcttgaaaaacaacagacaaatcaaactgattgccgcctccgtcgcagttgccgcatcctttcaggcacatgctggactgggcggactgaatatccagtccaaccttgacgaacccttttccggcagcattaccgtaaccggcgaagaagccaaagccctgctaggcggcggcagcgttaccgtttccgaaaaaggcctgaccgccaaagtccacaagttgggcgacaaagccgtcattgccgtttcttccgaacaggcagtccgcgatcccgtcctggtgttccgcatcggcgcaggcgcacaggtacgcgaatacaccgccatcctcgatcctgtcggctactcgcccaaaaccaaatctgcactttcagacggcaagacacaccgcaaaaccgctccgacagcagagtcccaagaaaatcaaaacgccaaagccctccgcaaaaccgataaaaaagacagcgcgaacgcagccgtcaaaccggcatacaacggcaaaacccataccgtccgcaaaggcgaaacggtcaaacagattgccgccgccatccgcccgaaacacctgacgctcgaacaggttgccgatgcgctgctgaaggcaaacccaaatgtttccgcacacggcagactgcgtgcgggcagcgtgcttcacattccgaatctgaacaggatcaaagcggaacaacccaaaccgcaaacggcgaaacccaaagccgaaaccgcatccatgccgtccgaaccgtccaaacaggcaacggtagagaaaccggttgaaaaacctgaagcaaaagttgccgcgcccgaagcaaaagcggaaaaaccggccgttcgacccgaacctgtacccgctgcaaatactgccgcatcggaaaccgctgccgaatccgccccccaagaagccgccgcttctgccatcgacacgccgaccgacgaaaccggtaacgccgtttccgaacctgtcgaacaggtttctgccgaagaagaaaccgaaagcggactgtttgacggtctgttcggcggttcgtacaccttgctgcttgccggcggaggcgcggcattaatcgccctgctgctgcttttgcgccttgcccaatccaaacgcgcgcgccgtaccgaagaatccgtccctgaggaagagcctgaccttgacgacgcggcagacgacggcatagaaatcacctttgccgaagtcgaaactccggcaacgcccgaacccgctccgaaaaacgatgtaaacgacacacttgccttagatggggaatctgaagaagagttatcggcaaaacaaacgttcgatgtcgaaaccgatacgccttccaaccgcatcgacttggatttcgacagcctggcagccgcgcaaaacggcattttatccggcgcacttacgcaggatgaagaaacccaaaaacgcgcggatgccgattggaacgccatcgaatccacagacagcgtgtacgagcccgagaccttcaacccgtacaaccctgtcgaaatcgtcatcgacacgcccgaaccggaatctgtcgcccaaactgccgaaaacaaaccggaaaccgtcgataccgatttctccgacaacctgccctcaaacaaccatatcggcacagaagaaacagcttccgcaaaacctgcctcaccctccggactggcaggcttcctgaaggcttcctcgcccgaaaccatcttggaaaaaacagttgccgaagtccaaacaccggaagagttgcacgatttcctgaaagtgtacgaaaccgatgccgtcgcggaaactgcgcctgaaacgcccgatttcaacgccgccgcagacgatttgtccgcattgcttcaacctgccgaagcaccgtccgttgaggaaaatataacggaaaccgttgccgaaacacccgacttcaacgccaccgcagacgatttgtccgcattacttcaaccttctaaagtacctgccgttgaggaaaatgcagcggaaaccgttgccgatgatttgtccgcactgttgcaacctgctgaagcaccggccgttgaggaaaatgtaacggaaaccgttgccgaaacacccgatttcaacgccaccgcagacgatttgtccgcattacttcaaccttctgaagcacctgccgttgaggaaaatgcagcggaaaccgttgccgatgatttgtccgcactgttgcaacctgctgaagcaccggccgttgaggaaaatgcagcggaaatcactttggaaacgcctgattccaacacctctgaggcagacgctttgcccgacttcctgaaagacggcgaggaggaaacggtagattggagcatctacctctcggaagaaaatatcccaaataatgcagataccagtttcccttcggaatctgtaggttctgacgcgccttccgaagcgaaatacgaccttgccgaaatgtatctcgaaatcggcgaccgcgatgccgctgccgagacagtgcagaaattgctggaagaagcggaaggcgacgtactcaaacgtgcccaagcattggcgcaggaattgggtatttga

SEQ ID NO: 2 - protein, 875 aa, Neisseria meningitidis (group B)
MPAGRLPRRCPMMTKFTDCTRSNRIQPPTHRGYILKNNRQIKLIAASVAVAASFQAHAGLGGLNIQSNLDEPFSGSITVTGEEAKALLGGGSVTVSEKGLTAKVHKLGDKAVIAVSSEQAVRDPVLVFRIGAGAQVREYTAILDPVGYSPKTKSALSDGKTHRKTAPTAESQENQNAKALRKTDKKDSANAAVKPAYNGKTHTVRKGETVKQIAAAIRPKHLTLEQVADALLKANPNVSAHGRLRAGSVLHIPNLNRIKAEQPKPQTAKPKAETASMPSEPSKQATVEKPVEKPEAKVAAPEAKAEKPAVRPEPVPAANTAASETAAESAPQEAAASAIDTPTDETGNAVSEPVEQVSAEEETESGLFDGLFGGSYTLLLAGGGAALIALLLLLRLAQSKRARRTEESVPEEEPDLDDAADDGIEITFAEVETPATPEPAPKNDVNDTLALDGESEEELSAKQTFDVETDTPSNRIDLDFDSLAAAQNGILSGALTQDEETQKRADADWNAIESTDSVYEPETFNPYNPVEIVIDTPEPESVAQTAENKPETVDTDFSDNLPSNNHIGTEETASAKPASPSGLAGFLKASSPETILEKTVAEVQTPEELHDFLKVYETDAVAETAPETPDFNAAADDLSALLQPAEAPSVEENITETVAETPDFNATADDLSALLQPSKVPAVEENAAETVADDLSALLQPAEAPAVEENVTETVAETPDFNATADDLSALLQPSEAPAVEENAAETVADDLSALLQPAEAPAVEENAAEITLETPDSNTSEADALPDFLKDGEEETVDWSIYLSEENIPNNADTSFPSESVGSDAPSEAKYDLAEMYLEIGDRDAAAETVQKLLEEAEGDVLKRAQALAQELGI

SEQ ID NO: 3 - DNA, 483 bp, Neisseria meningitidis (group B)
atggaaaggaacggtgtatttggtaaaattgtcggcaatcgcatactccgtatgtcgtccgaacacgctgccgcatcctatccgaaaccgtgcaaatcgtttaaactagcgcaatcttggttcagagtgcgaagctgtctgggcggcgtttttatttacggagcaaacatgaaacttatctataccgtcatcaaaatcattatcctgctgctcttcctgctgcttgccgtcattaatacggatgccgttaccttttcctacctgccggggcaaaaattcgatttgccgctgattgtcgtattgttcggcgcatttgtagtcggtattatttttggaatgtttgccttgttcggacggttgttgtcgttacgtggcgagaacggcaggttgcgtgccgaagtaaagaaaaatgcgcgtttgacggggaaggagctgaccgcaccaccggcgcaaaatgcgcccgaatctaccaaacagccttaa

SEQ ID NO: 4 - protein, 160 aa, Neisseria meningitidis (group B)
MERNGVFGKIVGNRILRMSSEHAAASYPKPCKSFKLAQSWFRVRSCLGGVFIYGANMKLIYTVIKIIILLLFLLLAVINTDAVTFSYLPGQKFDLPLIVVLFGAFVVGIIFGMFALFGRLLSLRGENGRLRAEVKKNARLTGKELTAPPAQNAPESTKQP

SEQ ID NO: 5 - DNA, 1551 bp, Neisseria meningitidis (group B)
atgaaaaaacctttgatttcggttgcggcagcattgctcggcgttgctttgggcacgccttattatttgggtgtcaaagccgaagaaagcttgacgcagcagcaaaaaatattgcaggaaacgggcttcttgaccgtcgaatcgcaccaatatgagcgcggctggtttacctctatggaaacgacggtcatccgtctgaaacccgagttgctgaataatgcccgaaaatacctgccggataacctgaaaacagtgttggaacagccggttacgctggttaaccatatcacgcacggccctttcgccggcggattcggcacgcaggcgtacattgaaaccgagttcaaatacgcgcctgaaacggaaaaagttctggaacgcttttttggaaaacaagtcccggcttcccttgccaataccgtttattttaacggcagcggtaaaatggaagtcagtgttcccgccttcgattatgaagagctgtcgggcatcaggctgcactgggaaggcctgacgggagaaacggtttatcaaaaaggtttcaaaagctaccggaacggctatgatgcccccttgtttaaaatcaagctggcagacaaaggcgatgccgcgtttgaaaaagtgcatttcgattcggaaacttcagacggcatcaatccgcttgctttgggcagcagcaatctgaccttggaaaaattctccctagaatggaaagagggtgtcgattacaacgtcaagttaaacgaactggtcaatcttgttaccgatttgcagattggcgcgtttatcaatcccaacggcagcatcgcaccttccaaaatcgaagtcggcaaactggctttttcaaccaagaccggggaatcaggcgcgtttatcaacagtgaagggcagttccgtttcgatacactggtgtacggcgatgaaaaatacggcccgctggacatccatatcgctgccgaacacctcgatgcttctgccttaaccgtattgaaacgcaagtttgcacaaatttccgccaaaaaaatgaccgaggaacaaatccgcaatgatttgattgccgccgtcaaaggagaggcttccggactgttcaccaacaatcccgtattggacattaaaactttccgattcacgctgccatcgggaaaaatcgatgtgggcggaaaaatcatgtttaaagacatgaagaaggaagatttgaatcaattgggtttgatgctgaagaaaaccgaagccgacatcagaatgagtattccccaaaaaatgctggaagacttggcggtcagtcaagcaggcaatattttcagcgtcaatgccgaagatgaggcggaaggcagggcaagtcttgacgacatcaacgagaccttgcgcctgatggtggacagtacggttcagagtatggcaagggaaaaatatctgactttgaacggcgaccagattgatactgccatttctctgaaaaacaatcagttgaaattgaacggtaaaacgttgcaaaacgaaccggagccggattttgatgaaggcggtatggtttcagagccgcagcagtaa

SEQ ID NO: 6 - protein, 516 aa, Neisseria meningitidis (group B)
MKKPLISVAAALLGVALGTPYYLGVKAEESLTQQQKILQETGFLTVESHQYERGWFTSMETTVIRLKPELLNNARKYLPDNLKTVLEQPVTLVNHITHGPFAGGFGTQAYIETEFKYAPETEKVLERFFGKQVPASLANTVYFNGSGKMEVSVPAFDYEELSGIRLHWEGLTGETVYQKGFKSYRNGYDAPLFKIKLADKGDAAFEKVHFDSETSDGINPLALGSSNLTLEKFSLEWKEGVDYNVKLNELVNLVTDLQIGAFINPNGSIAPSKIEVGKLAFSTKTGESGAFINSEGQFRFDTLVYGDEKYGPLDIHIAAEHLDASALTVLKRKFAQISAKKMTEEQIRNDLIAAVKGEASGLFTNNPVLDIKTFRFTLPSGKIDVGGKIMFKDMKKEDLNQLGLMLKKTEADIRMSIPQKMLEDLAVSQAGNIFSVNAEDEAEGRASLDDINETLRLMVDSTVQSMAREKYLTLNGDQIDTAISLKNNQLKLNGKTLQNEPEPDFDEGGMVSEPQQ

SEQ ID NO: 7 - DNA, 855 bp, Neisseria meningitidis (group B)
atgaagatcgtcctgattagcggcctgtccggttcgggcaagtccgtcgcactgcgccaaatggaagattcgggttatttctgcgtggacaatttgcctttggaaatgttgcccgcgctggtgtcgtatcatatcgaacgtgcggacgaaaccgaattggcggtcagcgtcgatgtgcgttccggcattgacatcggacaggcgcgggaacagattgcctctctgcgcagactggggcacagggttgaagttttgtttgtcgaggcggaagaaagcgtgttggtccgccggttttccgaaaccaggcgaggacatcctctgagcaatcaggatatgaccttgttggaaagcttaaagaaagaacgggaatggctgttcccgcttaaagaaatcgcctattgtatcgacacttccaagatgaatgcccaacagctccgccatgcagtccggcagtggctgaaggtcgaacgtaccgggctgctggtgattttggagtccttcgggttcaaatacggtgtgccgaacaacgcggattttatgttcgatatgcgcagcctgcccaacccgtattacgatcccgagttgaggccttacaccggtatggacaagcccgtttgggattatttggacggacagccgcttgtgcaggaaatggttgacgacatcgaaaggtttgttacgcattggttaccgcgtttggaggatgaaagcaggagctacgttaccgtcgccatcggttgcacgggaggacagcaccgttcggtctatattgtcgaaaaactcgcccgaaggttgaaagggcgttatgaattgctgatacggcacagacaggcgcaaaacctgtcagaccgctaa

SEQ ID NO: 8 - protein, 284 aa, Neisseria meningitidis (group B)
MKIVLISGLSGSGKSVALRQMEDSGYFCVDNLPLEMLPALVSYHIERADETELAVSVDVRSGIDIGQAREQIASLRRLGHRVEVLFVEAEESVLVRRFSETRRGHPLSNQDMTLLESLKKEREWLFPLKEIAYCIDTSKMNAQQLRHAVRQWLKVERTGLLVILESFGFKYGVPNNADFMFDMRSLPNPYYDPELRPYTGMDKPVWDYLDGQPLVQEMVDDIERFVTHWLPRLEDESRSYVTVAIGCTGGQHRSVYIVEKLARRLKGRYELLIRHRQAQNLSDR

SEQ ID NO: 9 - DNA, 1416 bp, Neisseria meningitidis (group B)
atgaacctgcatgcaaaggacaaaacccagcatcccgaaaacgtcgagctgctcagtgcgcagaagccgattaccgactttaagggcctgctgaccaccattatttccgccgtcgtctgtttcggcatttaccacatcctgccttacagccccgatgccaataaaggtatcgcgctgctgattttcgttgccgcactttggtttaccgaggccgtccacattaccgtaaccgcactgatggtgccgattctcgccgtcgtactcggtttccccgacatggacatcaaaaaggcgatggctgatttttccaacccgattatctacatttttttcggcggcttcgcgcttgccaccgccctgcatatgcagcggctggaccgtaaaatcgccgtcagcctgttgcgcctgtcgcgcggcaatatgaaagtggcggttttgatgttgttcctcgttaccgcctttctgtccatgtggatcagcaacaccgccaccgccgcgatgatgctgcctctagcaatgggtatgctgagccacctcgaccaggaaaaagaacacaaaacctacgtcttcctcctgctcggcatcgcctattgcgccagcatcggcggcttgggcacgctcgtcggctcgccgcccaacctgattgccgccaaagccctaaatctggacttcgtcggctggatgaagctcggcctgccgatgatgctgttgattctgcccttgatgctgctctccctgtacgtcatcctcaaacctaatttgaacgaacgcgtggaaatcaaagccgaatccatcccttggacgctgcaccgcgtgatcgcgctgttgattttccttgccacagccgccgcgtggatattcagctccaaaatcaaaaccgccttcggcatttccaatcccgacaccgttatcgccctgagtgccgccgtcgccgtcgtcgtcttcggcgtggcgcaatggaaggaagtcgcccgcaataccgactggggcgtgttgatgctcttcggcggcggcatcagcctgagcacgctgttgaaaacatccggcgcgtccgaagccttgggacagcaggttgccgccaccttttccggcgcgcccgcatttttggtgatactcatcgtcgccgccttcattatttttctgaccgagttcaccagcaacaccgcctccgccgcattgcttgtaccgattttctccggcatcgctatgcagatggggctgcccgaacaagtcttggtattcgtcatcggcatcggcgcatcttgtgccttcatgctgccggttgccacaccgcctaacgcgattgtgttcggcacgggcttaatcaagcaacgcgaaatgatgaatgtcggcatactgctgaacatcctctgcgtagtattggttgctctgtgggcttatgctgtactgatgtaa

SEQ ID NO: 10 - protein, 471 aa, Neisseria meningitidis (group B)
MNLHAKDKTQHPENVELLSAQKPITDFKGLLTTIISAVVCFGIYHILPYSPDANKGIALLIFVAALWFTEAVHITVTALMVPILAVVLGFPDMDIKKAMADFSNPIIYIFFGGFALATALHMQRLDRKIAVSLLRLSRGNMKVAVLMLFLVTAFLSMWISNTATAAMMLPLAMGMLSHLDQEKEHKTYVFLLLGIAYCASIGGLGTLVGSPPNLIAAKALNLDFVGWMKLGLPMMLLILPLMLLSLYVILKPNLNERVEIKAESIPWTLHRVIALLIFLATAAAWIFSSKIKTAFGISNPDTVIALSAAVAVVVFGVAQWKEVARNTDWGVLMLFGGGISLSTLLKTSGASEALGQQVAATFSGAPAFLVILIVAAFIIFLTEFTSNTASAALLVPIFSGIAMQMGLPEQVLVFVIGIGASCAFMLPVATPPNAIVFGTGLIKQREMMNVGILLNILCVVLVALWAYAVLM

SEQ ID NO: 11 - DNA, 1005 bp, Neisseria meningitidis (group B)
atgcaacgacaaatcaaactgaaaaattggcttcagaccgtttatcccgaacgggacttcgatctgacttttgcggcggcggatgctgatttccgccgctatttccgtgcaacgttttcagacggcagcagtgtcgtctgcatggatgcaccgcccgacaagatgagtgtcgcaccttatttgaaagtgcagaaactgtttgacatggtcaatgtgccgcaggtattgcacgcggacacggatctggggtttgtggtattgaacgacttgggcaatacgacgtttttgaccgcaatgcttcaggaacagggcgaaacggcgcacaaagccctgcttttggaggcaatcggcgagttggtcgaattgcagaaggcgagccgtgaaggggttttgcccgaatatgaccgtgaaacgatgttgcgcgaaatcaacctgttcccggaatggtttgtcgcaaaagaattggggcgcgaattaacattcaaacaacgccaactttggcagcaaaccgtcgatacgctgctgccgcccctgttggcgcagcccaaagtctatgtgcaccgcgactttatcgtccgcaacctgatgctgacgcgcggcaggccgggcgttttagacttccaagacgcgctttacggcccgatttcctacgatttggtgtcgctgttgcgcgatgcctttatcgaatgggaagaagaatttgtcttggacttggttatccgctactgggaaaaggcgcgggctgccggcttgcccgtccccgaagcgtttgacgagttttaccgctggttcgaatggatgggcgtgcagcggcacttgaaggttgcaggcatcttcgcacgcctgtactaccgcgacggcaaagacaaataccgtccggaaatcccgcgtttcttaaactatctgcgccgcgtatcgcgccgttatgccgaactcgccccgctctacgcgctcttggtcgaactggtcggcgatgaagaactggaaacgggctttacgttttaa

SEQ ID NO: 12 - protein, 334 aa, Neisseria meningitidis (group B)
MQRQIKLKNWLQTVYPERDFDLTFAAADADFRRYFRATFSDGSSVVCMDAPPDKMSVAPYLKVQKLFDMVNVPQVLHADTDLGFVVLNDLGNTTFLTAMLQEQGETAHKALLLEAIGELVELQKASREGVLPEYDRETMLREINLFPEWFVAKELGRELTFKQRQLWQQTVDTLLPPLLAQPKVYVHRDFIVRNLMLTRGRPGVLDFQDALYGPISYDLVSLLRDAFIEWEEEFVLDLVIRYWEKARAAGLPVPEAFDEFYRWFEWMGVQRHLKVAGIFARLYYRDGKDKYRPEIPRFLNYLRRVSRRYAELAPLYALLVELVGDEELETGFTF

SEQ ID NO: 13 - DNA, 1785 bp, Neisseria meningitidis (group B)
atggaactgatgactgttttgctgcctttggcggcgttggtgtcgggcgtgttgtttacatggttgctgatgaagggccggtttcagggcgagtttgccggtttgaacgcgcacctggcggaaaaggcggcaagatgtgattttgtcgaacaggcacacggcaaaaccgtgtcggaattggcggtgttggacgggaaataccggcatttgcaggacgaaaattatgctttgggcaaccgtttttccgcagccgaaaagcagattgcccatttgcaggaaaaagaggcggagtcggcgcggctgaagcagtcgtatatcgagttgcaggaaaaggcacagggtttggcggttgaaaacgaacgtttggcaacgcagctcggacaggaacggaaggcgtttgccgaccaatatgccttggaacgccaaatccgccaaagaatcgaaaccgatttggaagaaagccgccaaactgtccgcgacgtgcaaaacgacctttccgatgtcggcaaccgttttgccgcagccgaaaaacagattgcccatttgcaggaaaaagaggcggaagcggagcggttgaggcagtcgcataccgagttgcaggaaaaggcacagggtttggcggttgaaaacgaacgtttggcaacgcaaatcgaacaggaacgccttgcttctgaagagaagctgtccttgctgggcgaggcgcgcaaaagtttgagcgatcagtttcaaaatcttgccaacacgattttggaagaaaaaagccgccgttttaccgagcagaaccgcgagcagctccatcaggttttgaacccgctaaacgaacgcatccacggtttcggcgagttggtcaagcaaacctatgataaagaatcgcgcgagcggctgacgttggaaaacgaattgaaacggcttcaggggttgaacgcgcagctgcacagcgaggcaaaggccctgaccaacgcgctgaccggtacgcagaataaggttcagggcaattggggcgagatgattctggaaacggttttggaaaattccggccttcagaaagggcgggaatatgtggttcaggcggcatccgtccgaaaagaggaagacggcggcacgcgccgcctccagcccgacgttttggtcaacctgcccgacaacaagcagattgtgattgattccaaggtctcgctgacagcttatgtgcgctacacgcaggcggcggatgcggatacggcggcacgcgaactggcggcacacgttgccagcatccgtgcacacatgaaaggcttgtcgctgaaggattacaccgatttggaaggtgtgaacacattggatttcgtctttatgtttatccctgtcgaaccggcctacctgttggcgttgcagaatgacgcgggcttgttccaagagtgtttcgacaaacggattatgctggtcggccccagtacgctgctggcgactttgaggacggtggcgaatatttggcgcaacgaacagcaaaatcagaacgcactggcgattgcggacgaaggcggcaagctgtacgacaagtttgtcggcttcgtacagacgctcgaaagcgtcggcaaaggcatcgatcaggcgcaaagcagttttcagacggcattcaagcaacttgccgaagggcgcgggaatctggtcggacgcgccgagaaactgcgtctgttgggcgtgaaggcaggcaaacaacttcaacgggatttggtcgagcgttccaatgaaacaacggcgttgtcggaatctttggaatacgcggcagaagatgaagcagtctga

SEQ ID NO: 14 - protein, 594 aa, Neisseria meningitidis (group B)
MELMTVLLPLAALVSGVLFTWLLMKGRFQGEFAGLNAHLAEKAARCDFVEQAHGKTVSELAVLDGKYRHLQDENYALGNRFSAAEKQIAHLQEKEAESARLKQSYIELQEKAQGLAVENERLATQLGQERKAFADQYALERQIRQRIETDLEESRQTVRDVQNDLSDVGNRFAAAEKQIAHLQEKEAEAERLRQSHTELQEKAQGLAVENERLATQIEQERLASEEKLSLLGEARKSLSDQFQNLANTILEEKSRRFTEQNREQLHQVLNPLNERIHGFGELVKQTYDKESRERLTLENELKRLQGLNAQLHSEAKALTNALTGTQNKVQGNWGEMILETVLENSGLQKGREYVVQAASVRKEEDGGTRRLQPDVLVNLPDNKQIVIDSKVSLTAYVRYTQAADADTAARELAAHVASIRAHMKGLSLKDYTDLEGVNTLDFVFMFIPVEPAYLLALQNDAGLFQECFDKRIMLVGPSTLLATLRTVANIWRNEQQNQNALAIADEGGKLYDKFVGFVQTLESVGKGIDQAQSSFQTAFKQLAEGRGNLVGRAEKLRLLGVKAGKQLQRDLVERSNETTALSESLEYAAEDEAV

SEQ ID NO: 15 - DNA, 561 bp, Neisseria meningitidis (group B)
atgaacagactgctactgctgtctgccgccgtcctgctgactgcctgcggcagcggcgaaaccgataaaatcggacgggcaagtaccgttttcaacatactgggcaaaaacgaccgtatcgaagtggaaggattcgacgatcccgacgttcaaggggttgcctgttatatttcgtatgcaaaaaaaggcggcttgaaggaaatggtcaatttggaagaggacgcgtccgacgcatcggtttcgtgcgttcagacggcatcttcgatttcttttgacgaaaccgccgtgcgcaaaccgaaagaagttttcaaacacggtgcgagcttcgcgttcaagagccggcagattgtccgttattacgaccccaaacgcaaaaccttcgcctatttggtgtacagcgataaaatcatccaaggctcgccgaaaaattccttaagcgcggtttcctgtttcggcggcggcataccgcaaaccgatggggtgcaagccgatacttccggcaacctgcttgccggcgcctgcatgatttccaacccgatagaaaatctcgacaaacgctga

SEQ ID NO: 16 - protein, 186 aa, Neisseria meningitidis (group B)
MNRLLLLSAAVLLTACGSGETDKIGRASTVFNILGKNDRIEVEGFDDPDVQGVACYISYAKKGGLKEMVNLEEDASDASVSCVQTASSISFDETAVRKPKEVFKHGASFAFKSRQIVRYYDPKRKTFAYLVYSDKIIQGSPKNSLSAVSCFGGGIPQTDGVQADTSGNLLAGACMISNPIENLDKR

SEQ ID NO: 17 - DNA, 693 bp, Neisseria meningitidis (group B)
atgaccgcctttgtccacaccctttcagacggcatggaactgaccgtcgaaatcaagcgccgtgccaagaaaaacctgattatccgccccgccggcacacataccgtccgcatcagcgtcccaccctgcttctccgtctccgctctaaaccgctggctgtatgaaaacgaagccgtcctgcggcaaacactggcgaaaacaccgccgccgcaaactgccgaaaaccggctgcccgaatccatcctcttccacggcagacagcttgccctcaccgcccatcaagacacgcaaatcctgctgatgccgtctgaaatccgtgttcccgaaggcgcacccgaaaaacagcttgcgctgctgcgggactttttggaacggcaggcgcacagttacctgattccccgcctcgaacgccacgcccgcaccacacaactgttccccgcctcctcctcgctgacctctgccaaaaccttctggggcgtgtgccgcaaaaccacaggcatacgcttcaactggcggctggtcggcgcaccggaatacgttgccgactatgtctgcatacacgaactctgccacctcgcccatcccgaccacagccccgccttttgggaactgacccgccgcttcgccccctacacgcccaaagcgaaacagtggctcaaaatccacggcagggaacttttcgccttaggctga

SEQ ID NO: 18 - protein, 230 aa, Neisseria meningitidis (group B)
MTAFVHTLSDGMELTVEIKRRAKKNLIIRPAGTHTVRISVPPCFSVSALNRWLYENEAVLRQTLAKTPPPQTAENRLPESILFHGRQLALTAHQDTQILLMPSEIRVPEGAPEKQLALLRDFLERQAHSYLIPRLERHARTTQLFPASSSLTSAKTFWGVCRKTTGIRFNWRLVGAPEYVADYVCIHELCHLAHPDHSPAFWELTRRFAPYTPKAKQWLKIHGRELFALG

SEQ ID NO: 19 - DNA, 1257 bp, Neisseria meningitidis (group B)
atgaacgccgcacaactcgaccataccgccaaagttttggctgaaatgctgactttcaaacagcctgccgatgccgtcctctccgcctatttccgcgaacacaaaaagctcggcagtcaagatcgccacgaaatcgccgaaaccgcctttgccgcgctgcgccactatcaaaaaatcagtaccgccctacgccgtccgcacgcgcagccgcgcaaagccgctctcgccgcactggttctcggcagaagcaccaacatcagccaaatcaaagacctgcttgatgaagaagaaacagcgttcctcggcaatttgaaagcccgtaaaaccgagttttcagacagcctgaataccgccgcagaattgccgcaatggctggtggaacaactgaaacagcattggcgcgaagaagaaatcctcgctttcggccgcagcatcaaccagcctgccccgctcgacatccgcgtcaacactttgaaaggcaaacgcgataaagtgctgccgctgttgcaagccgaaagtgccgatgcagaggcaacgccttattcgccttggggcatccgcctgaaaaacaaaatcgcgcttaacaaacacgaactgtttttagacggcacactggaagtccaagacgaaggcagccagctgcttgccttattggtgggcgcaaaacgaggcgaaatcattgtcgatttctgtgccggtgccggcggtaaaaccttggctgtcggtgcgcaaatggcgaacaaaggcagaatctacgccttcgatatcgccgaaaaacgccttgccaacctcaaaccgcgtatgacccgcgccggactgaccaatatccaccccgaacgcatcggcagcgaacacgatgcccgtatcgcccgactggcaggcaaagccgaccgtgtgttggtggacgcgccctgctccggtttgggcactttacgccgcaatcccgacctcaaataccgccaatccgccgaaaccgtcgccaaccttttggaacagcaacacagcatcctcgatgccgcctccaaactggtaaaaccgcaaggacgtttggtgtacgccacttgcagcatcctgcccgaagaaaacgagctgcaagtcgaacgtttcctgtccgaacatcccgaatttgaacccgtcaactgcgccgaactgcttgccggtttgaaaatcgatttggataccggcaaatacctgcgcctcaactccgcccgacaccaaaccgacggcttcttcgccgccgtattgcaacgcaaataa

SEQ ID NO: 20 - protein, 418 aa, Neisseria meningitidis (group B)
MNAAQLDHTAKVLAEMLTFKQPADAVLSAYFREHKKLGSQDRHEIAETAFAALRHYQKISTALRRPHAQPRKAALAALVLGRSTNISQIKDLLDEEETAFLGNLKARKTEFSDSLNTAAELPQWLVEQLKQHWREEEILAFGRSINQPAPLDIRVNTLKGKRDKVLPLLQAESADAEATPYSPWGIRLKNKIALNKHELFLDGTLEVQDEGSQLLALLVGAKRGEIIVDFCAGAGGKTLAVGAQMANKGRIYAFDIAEKRLANLKPRMTRAGLTNIHPERIGSEHDARIARLAGKADRVLVDAPCSGLGTLRRNPDLKYRQSAETVANLLEQQHSILDAASKLVKPQGRLVYATCSILPEENELQVERFLSEHPEFEPVNCAELLAGLKIDLDTGKYLRLNSARHQTDGFFAAVLQRK

SEQ ID NO: 21 - DNA, 1014 bp, Neisseria meningitidis (group B)
atgcaagtctattacgataaagatgccgatctgtccctaatcaaaggcaaaaccgttgccatcatcggttacggttcgcaaggtcatgcccatgccgccaacctgaaagattcgggtgtaaacgtggtgattggtctgcgccaaggttcttcttggaaaaaagccgaagcagccggtcatgtcgtcaaaaccgttgctgaagcgaccaaagaagccgatgtcgttatgctgctgctgcctgacgaaaccatgcctgccgtctatcacgccgaagttacagccaatttgaaagaaggcgcaacgctggcatttgcacacggcttcaacgtgcactacaaccaaatcgttccgcgtgccgacttggacgtgattatggttgcccccaaaggtccgggccataccgtacgcagtgaatacaaacgcggcggcggcgtgccttctctgattgccgtttaccaagacaattccggcaaagccaaagacatcgccctgtcttatgcggctgccaacggcggcaccaaaggcggtgtgattgaaaccactttccgcgaagaaaccgaaaccgatctgttcggcgaacaagccgtattgtgcggcggcgtggtcgagttgatcaaggcgggttttgaaaccctgaccgaagccggttacgcgcctgaaatggcttacttcgaatgtctgcacgaaatgaaactgatcgttgacctgattttcgaaggcggtattgccaatatgaactactccatttccaacaatgcggagtacggcgaatacgttaccggccctgaagtggtcaatgcttccagcaaagaagccatgcgcaatgccctgaaacgcattcaaaccggcgaatacgcaaaaatgtttatccaagagggtaatgtcaactatgcgtctatgactgcccgccgccgtctgaatgccgaccaccaagttgaaaaagtcggcgcacaactgcgtgccatgatgccttggattactgccaacaaattggttgaccaagacaaaaactga

SEQ ID NO: 22 - protein, 337 aa, Neisseria meningitidis (group B)
MQVYYDKDADLSLIKGKTVAIIGYGSQGHAHAANLKDSGVNVVIGLRQGSSWKKAEAAGHVVKTVAEATKEADVVMLLLPDETMPAVYHAEVTANLKEGATLAFAHGFNVHYNQIVPRADLDVIMVAPKGPGHTVRSEYKRGGGVPSLIAVYQDNSGKAKDIALSYAAANGGTKGGVIETTFREETETDLFGEQAVLCGGVVELIKAGFETLTEAGYAPEMAYFECLHEMKLIVDLIFEGGIANMNYSISNNAEYGEYVTGPEVVNASSKEAMRNALKRIQTGEYAKMFIQEGNVNYASMTARRRLNADHQVEKVGAQLRAMMPWITANKLVDQDKN

SEQ ID NO: 23 - DNA, 693 bp, Neisseria meningitidis (group B)
atgaaacttatcaaatacctgcaatatcaaggcataggaagccgcaagcagtgccaatggctgattgccggcggttatgttttcatcaacggaacctgcatggacgacaccgatgcagacatcgattcctcatccgtcgaaacgttggatattgacggggaagcagtaaccgtcgttcccgaaccctatttctacatcatgctcaacaagcctgaagattacgaaacttcgcacaaacccaagcactaccgcagcgtattcagcctgttccccgacaatatgcggaacatcgatatgcaggcggtcggcaggctggatgcagatacgaccggcgtattgctgattaccaacgacggcaaactgaaccacagcctgacttcgccgagcagaaaaattcccaagctgtacgaagtaacgctcaaacaccccacaggagaaacgctctgcgaaaccttgaaaaacggcgtgctgctccacgacgaaaacgaaaccgtttgtgccgccgatgccgttttgaaaaacccgaccaccctgctgctgaccattaccgaaggaaaataccaccaagtcaaacgcatgatcgccgccgccggcaaccgcgtgcaacaccttcatcgccggcgattcgcacatctggaaacagaaaacctcaaacccggggaatggaaatttatcgaatgtccaaaattctga

SEQ ID NO: 24 - protein, 230 aa, Neisseria meningitidis (group B)
MKLIKYLQYQGIGSRKQCQWLIAGGYVFINGTCMDDTDADIDSSSVETLDIDGEAVTVVPEPYFYIMLNKPEDYETSHKPKHYRSVFSLFPDNMRNIDMQAVGRLDADTTGVLLITNDGKLNHSLTSPSRKIPKLYEVTLKHPTGETLCETLKNGVLLHDENETVCAADAVLKNPTTLLLTITEGKYHQVKRMIAAAGNRVQHLHRRRFAHLETENLKPGEWKFIECPKF

SEQ ID NO: 25 - DNA, 900 bp, Neisseria meningitidis (group B)
atgaaaaccaattcagaagaactgaccgtatttgttcaagtggtggaaagcggcagcttcagccgtgcggcggagcagttggcgatggcaaattctgccgtaagccgcatcgtcaaacggctggaggaaaagttgggtgtgaacctgctcaaccgcaccacgcggcaactcagtctgacggaagaaggcgcgcaatatttccgccgcgcgcagagaatcctgcaagaaatggcagcggcggaaaccgaaatgctggcagtgcacgaaataccgcaaggcgtgttgagcgtggattccgcgatgccgatggtgctgcatctgctggcgccgctggcagcaaaattcaacgaacgctatccgcatatccgactttcgctcgtttcttccgaaggctatatcaatctgattgaacgcaaagtcgatattgccttacgggccggagaattggacgattccgggctgcgtgcacgccatctgtttgacagccgcttccgcgtaatcgccagtcctgaatacctggcaaaacacggcacgccgcaatctacagaagagcttgccggccaccaatgtttaggcttcaccgaacccggttctctaaatacatgggcggttttagatgcgcagggaaatccctataagatttcaccgcactttaccgccagcagcggtgaaatcttacgctcgttgtgcctttcaggttgcggtattgtttgcttatcagattttttggttgacaacgacatcgctgaaggaaagttaattcccctgctcgccgaacaaacctccgataaaacacacccctttaatgctgtttattacagcgataaagccgtcaatctccgcttacgcgtatttttggattttttagtggaggaactgggaaacaatctctgtggataa

SEQ ID NO: 26 - protein, 299 aa, Neisseria meningitidis (group B)
MKTNSEELTVFVQVVESGSFSRAAEQLAMANSAVSRIVKRLEEKLGVNLLNRTTRQLSLTEEGAQYFRRAQRILQEMAAAETEMLAVHEIPQGVLSVDSAMPMVLHLLAPLAAKFNERYPHIRLSLVSSEGYINLIERKVDIALRAGELDDSGLRARHLFDSRFRVIASPEYLAKHGTPQSTEELAGHQCLGFTEPGSLNTWAVLDAQGNPYKISPHFTASSGEILRSLCLSGCGIVCLSDFLVDNDIAEGKLIPLLAEQTSDKTHPFNAVYYSDKAVNLRLRVFLDFLVEELGNNLCG

SEQ ID NO: 27 - DNA, 696 bp, Neisseria meningitidis (group B)
atgatgaaggatttgaatttgagcaacagcctgttcaaaggctacaacgacaaacatggcttaatgatttgtggctatgaatggggttggagtaaagccgatgaggctgcttatgtagcaggtgaatacaaactccctgaaaacaaaatcgaccatacatttgcaaacaaatccctctatttcggagagcaggcaaaaaagtggcgttacgacaatacgataaaaaattggtttgaaatgtggggacaccccttagacgaaaatggattgggcggtgcatttgaaaaatccctggttcaaaccaactgggctgctacacagggcaacactatcgacaatcccgacaagttcacacaacccgagcacatcgataattttctctaccacatcgaaaaactgcgtccgaaagtcatcctcttcatgggcagcaggttggcggattttctgaacaaccaaaatgtactgccacgcttcgagcagttggtcggtaagcagaccaaaccgctggagacggtgcaaaaagaatttgacggtacacgtttcaatgtcaaattccaatcgtttgaagattgcgaagtcgtctgctttccccatcccagtgccagtcgcggtctatcttacgattacatcgccttgtttgcgcctgaaatgaaccggattttatcggactttaaaacaacacgcggattcaaataa

SEQ ID NO: 28 - protein, 231 aa, Neisseria meningitidis (group B)
MMKDLNLSNSLFKGYNDKHGLMICGYEWGWSKADEAAYVAGEYKLPENKIDHTFANKSLYFGEQAKKWRYDNTIKNWFEMWGHPLDENGLGGAFEKSLVQTNWAATQGNTIDNPDKFTQPEHIDNFLYHIEKLRPKVILFMGSRLADFLNNQNVLPRFEQLVGKQTKPLETVQKEFDGTRFNVKFQSFEDCEVVCFPHPSASRGLSYDYIALFAPEMNRILSDFKTTRGFK

SEQ ID NO: 29 - DNA, 1065 bp, Neisseria meningitidis (group B)
atggaaaaagaattcaggatattaaatatcgtatcggccaagatttggggtggaggcgaacaatatgtctatgatgtttcaaaagcattggggcttcggggctgcacaatgtttaccgccgtcaataaaaataatgaattgatgcacaggcgattttccgaagtttcttccgttttcacaacgcgccttcacacgctcaacgggctgttttcgctctacgcacttacccgctttatccggaaaaaccgcatttcccacctgatgatacacaccggcaaaattgccgccttatccatacttttgaaaaaactgaccggggtgcgcctgatatttgtcaaacataatgtcgtcgccaacaaaaccgatttttaccaccgcctgatacagaaaaacacagaccgctttatttgcgtttcccgtctggtttacgatgtgcaaaccgccgacaatccctttaaagaaaaataccggattgttcataacggtatcgataccggccgtttccctccctctcaagaaaaacccgacagccgtttttttaccgtcgcctacgccggcaggatcagtccagaaaaaggattggaaaacctgattgaagcctgtgtgatactgcatcggaaatatcctcaaatcaggctcaaattggcaggggacggacatccggattatatgtgccgcctgaagcgggacgtatctgcttcaggagcagaaccatttgtttcttttgaagggtttaccgaaaaacttgcttcgttttaccgccaaagcgatgtcgtggttttgcccagcctcgtcccggaggcattcggtttgtcattatgcgaggcgatgtactgccgaacggcggtgatttccaatactttgggggcgcaaaaggaaattgtcgaacatcatcaatcggggattctgctggacaggctgacacctgaatctttggcggacgaaatcgaacgcctcgtcttgaaccctgaaacgaaaaacgcactggcaacggcagctcatcaatgcgtcgccgcccgttttaccatcaaccataccgccgacaaattattggatgcaatataa

SEQ ID NO: 30 - protein, 354 aa, Neisseria meningitidis (group B)
MEKEFRILNIVSAKIWGGGEQYVYDVSKALGLRGCTMFTAVNKNNELMHRRFSEVSSVFTTRLHTLNGLFSLYALTRFIRKNRISHLMIHTGKIAALSILLKKLTGVRLIFVKHNVVANKTDFYHRLIQKNTDRFICVSRLVYDVQTADNPFKEKYRIVHNGIDTGRFPPSQEKPDSRFFTVAYAGRISPEKGLENLIEACVILHRKYPQIRLKLAGDGHPDYMCRLKRDVSASGAEPFVSFEGFTEKLASFYRQSDVVVLPSLVPEAFGLSLCEAMYCRTAVISNTLGAQKEIVEHHQSGILLDRLTPESLADEIERLVLNPETKNALATAAHQCVAARFTINHTADKLLDAI

SEQ ID NO: 31 - DNA, 1272 bp, Neisseria meningitidis (group B)
atgcaggaacagaatcggaaaccaagttttcccatagtgatgttgctggtgtcggttgccctgtggatagcgtctttatccaatgttgcattttatttgggcaatcatggaagcatggagggtttgaccgttttgattttggggtcgatatttgcttctttggatatcaggtattgtgcggtctatgcgaattatgtttggttggcggccattgttttgctggcgttgcggaagaaggtcgtgcctgtccatgcggcactttggggcttggcgttggtggctttcagtgtgaaagccgtatacgtcgatgaagcagggaatacatcggatattgtgcgctacggtgcaggattttatttgtggtatgccgcatttgcggttgccaccatcggtacgtttgccggaaagaataaggaaagaaaagccgcatcagcggcagacgggataaaaatgacgtttgataaatggttgggcttgtcaaaactgcctaaaaatgaagcaagaatgctgctacaatatgtttcggaatatacgcgcgtgcagttgttgacgcggggcggggaagaaatgccggacgaagtccgacagcgggcggacaggctggcgcaacgccgtctgaacggcgagccggttgcctatattttaggtgtgcgcgaattttatggcagacgctttacagtcaatccgagcgtgctgattccgcgccccgaaaccgaacatttggtcgaagccgtattggcgcgcctgcccgaaaacgggcgcgtgtgggatttggggacgggcagcggcgcggttgccgtaaccgtcgcgctcgaacgccccgatgcgtttgtgcgcgcatccgacatcagcccgcccgcccttgaaacggcgcggaaaaatgcggcggatttgggcgcgcgggtcgaatttgcacacggttcgtggttcgacaccgatatgccgtctgaagggaaatgggacatcatcgtgtccaacccgccctatatcgaaaacggcgataaacatttgttgcaaggcgatttgcggtttgagccgcaaatcgcgctgaccgacttttcagacggcctaagctgcatccgcaccttggcgcaaggcgcgcccgaccgtttggcggaaggcggttttttattgctggaacacggtttcgatcagggcgcggcggtgcgcggcgtgttggcggagaatggtttttcaggagtggaaaccctgccggatttggcgggtttggacagggttacgctggggaagtatatgaagcatttgaaataa

SEQ ID NO: 32 - protein, 423 aa, Neisseria meningitidis (group B)
MQEQNRKPSFPIVMLLVSVALWIASLSNVAFYLGNHGSMEGLTVLILGSIFASLDIRYCAVYANYVWLAAIVLLALRKKVVPVHAALWGLALVAFSVKAVYVDEAGNTSDIVRYGAGFYLWYAAFAVATIGTFAGKNKERKAASAADGIKMTFDKWLGLSKLPKNEARMLLQYVSEYTRVQLLTRGGEEMPDEVRQRADRLAQRRLNGEPVAYILGVREFYGRRFTVNPSVLIPRPETEHLVEAVLARLPENGRVWDLGTGSGAVAVTVALERPDAFVRASDISPPALETARKNAADLGARVEFAHGSWFDTDMPSEGKWDIIVSNPPYIENGDKHLLQGDLRFEPQIALTDFSDGLSCIRTLAQGAPDRLAEGGFLLLEHGFDQGAAVRGVLAENGFSGVETLPDLAGLDRVTLGKYMKHLK

SEQ ID NO: 33 - DNA, 1170 bp, Neisseria meningitidis (group B)
atggacaacgaattgtggattatcctgctgccgattatccttttgcccgtcttcttcgcgatgggctggtttgccgcccgcgtggatatgaaaaccgtattgaagcaggcaaaaagcatcccttcgggattttataaaagcttggacgctttggtcgaccgcaacagcgggcgcgcggcaagggagttggcggaagtcgtcgacggccggccgcaatcgtatgatttgaacctcaccctcggcaaactttaccgccagcgtggcgaaaacgacaaagccatcaacatacaccggacaatgctcgattctcccgatacggtcggcgaaaagcgcgcgcgcgtcctgtttgaattggcgcaaaactaccaaagtgcggggttggtcgatcgtgccgaacagatttttttggggctgcaagacggtaaaatggcgcgtgaagccagacagcacctgctcaatatctaccaacaggacagggattgggaaaaagcggttgaaaccgcccggctgctcagccatgacgatcagacctatcagtttgaaatcgcccagttttattgcgaacttgcccaagccgcgctgttcaagtccaatttcgatgtcgcgcgtttcaatgtcggcaaggcactcgaagccaacaaaaaatgcacccgcgccaacatgattttgggcgacatcgaacaccgacaaggcaatttccctgccgccgtcgaagcctatgccgccatcgagcagcaaaaccatgcatacttgagcatggtcggcgagaagctttacgaagcctatgccgcgcagggaaaacctgaagaaggcttgaaccgtctgacaggatatatgcagacgtttcccgaacttgacctgatcaatgtcgtgtacgagaaatccctgctgcttaagtgcgagaaagaagccgcgcaaaccgccgtcgagcttgtccgccgcaagcccgaccttaacggcgtgtaccgcctgctcggtttgaaactcagcgatatgaatccggcttggaaagccgatgccgacatgatgcgttcggttatcggacggcagctacagcgcagcgtgatgtaccgttgccgcaactgccacttcaaatcccaagtctttttctggcactgccccgcctgcaacaaatggcagacgtttaccccgaataaaatcgaagtttaa

SEQ ID NO: 34 - protein, 389 aa, Neisseria meningitidis (group B)
MDNELWIILLPIILLPVFFAMGWFAARVDMKTVLKQAKSIPSGFYKSLDALVDRNSGRAARELAEVVDGRPQSYDLNLTLGKLYRQRGENDKAINIHRTMLDSPDTVGEKRARVLFELAQNYQSAGLVDRAEQIFLGLQDGKMAREARQHLLNIYQQDRDWEKAVETARLLSHDDQTYQFEIAQFYCELAQAALFKSNFDVARFNVGKALEANKKCTRANMILGDIEHRQGNFPAAVEAYAAIEQQNHAYLSMVGEKLYEAYAAQGKPEEGLNRLTGYMQTFPELDLINVVYEKSLLLKCEKEAAQTAVELVRRKPDLNGVYRLLGLKLSDMNPAWKADADMMRSVIGRQLQRSVMYRCRNCHFKSQVFFWHCPACNKWQTFTPNKIEV

SEQ ID NO: 35 - DNA, 3606 bp, Neisseria meningitidis (group B)
atgtttcattttgcatttccggcacaaactgccctgcgccaagcgataaccgatgcctaccgccgtaatgaaatcgaagccgtacaggatatgttgcaacgtgcacagatgagcgacgaagagcgcaacgccgcctccgagcttgcccgccgtttggttacccaagtccgcgccggccgcaccaaagccggcggcgtggatgcgctgatgcacgagttttcactctccagcgaagaaggcatcgcgctgatgtgtctggcagaagccctgctgcgtatccccgacaacgccacgcgcgaccgcctgattgccgacaagatttcagacggcaactggaaaagccatttgaacaacagcccttccctcttcgtcaatgctgccgcctggggcctgctgattaccggcaaactgaccgccacaaacgacaaacaaatgagttccgcactcagccgcctgatcagcaaaggcggcgcaccgctcatccgccaaggcgtaaattacgccatgcggcttctgggcaaacagttcgtaaccggacagaccattgaagaagccctgcaaaacggcaaagaacgcgaaaaaatgggctaccgcttctccttcgatatgttgggcgaagccgcctacacccaagccgatgccgaccgctactaccgcgactatgtcgaagccatccacgccatcggcaaagatgcggcaggacaaggcgtttacgaaggtaacggtatttccgtcaaactttccgccatccatccgcgctactcgcgcacccaacacggccgcgtgatgggcgaactgttgccgcgcctgaaagagctgttccttttgggtaaaaaatacgatatcggtatcaacatcgatgccgaagaagccaaccgtctggagctgtctttggatttgatggaggctttggtttcagaccctgacttggctggctacaaaggtatcggtttcgttgtccaagcctaccaaaaacgttgtccgttcgttatcgactacctgatcgaccttgcccgccgcaacaaccaaaaactaatgatccgcctcgtcaaaggcgcgtattgggacagcgaaatcaaatgggcgcaagtggacggcttgaacggctatccgacctacacccgcaaagtccacaccgacatctcctacctcgcctgcgcgcgcaaactgctttccgcgcaagacgcggtattcccgcaatttgccacccacaacgcctacactttgggcgcaatctaccaaatgggtaaaggcaaagattttgaacaccaatgcctgcacggtatgggcgaaaccctgtacgaccaagtcgtcggcccgcaaaacttaggccgccgcgtgcgcgtgtacgccccagtcggcacacacgaaaccctgctcgcctacttggtgcgccgcctgttggaaaacggcgcgaactcgtctttcgtcaaccaaatcgtcgatgaaaacatcagcatcgacacgctcatccgcagcccgttcgacaccatcgccgaacaaggcatccacctgcacaacgccctgccgctgccgcgcgatttgtacggcaaatgccgtctgaactcgcaaggcgtggacttgagcaacgaaaacgtattgcagcagcttcaagaacagatgaacaaagccgccgcgcaagacttccacgccgcatccatcgtcaacggcaaagcccgcgatgtcggcgaagcgcaaccgattaaaaaccctgccgaccacgacgacatcgtcggcacagtcagctttgccgatgccgcgcttgcccaagaagcggttggcgcagccgttgccgcgttccccgaatggagtgcgacacctgccgccgaacgcgccgcctgcctgcgccgttttgccgatttgctggagcagcacaccccagcactgatgatgcttgccgtgcgcgaagcaggcaaaacgctgaacaacgccattgccgaagtgcgcgaagccgtcgatttctgccgctactacgcaaacgaagccgaacataccctgcctcaagacgcaaaagccgtcggcgcgattgtcgccatcagcccgtggaacttcccgctcgccatctttaccggcgaagtcgtttccgcattggcggcaggcaacaccgtcatcgccaaacccgccgaacaaaccagcctgattgccggttatgccgtttccctcatgcacgaagccggcatcccgacttccgccctgcaactcgtcctcggcgcaggcgacgtgggtgcggcattgaccaacgatgcccgcatcggcggcgtgattttcaccggctcgaccgaagtggcgcgcctgatcaacaaagcccttgccaaacgcggcgacaatcccgtcctgattgccgaaaccggcggacaaaacgccatgattgtcgattccaccgcacttgccgagcaagtctgcgccgacgtattgaactccgccttcgacagcgcgggacaacgctgctccgccctgcgcattttgtgcgtccaagaagacgttgccgaccgtatgctcgacatgatcaaaggcgctatggacgaactcgtcgtcggcaaaccgattcagctcactaccgatgtcggccccgtcatcgatgccgaagcacagcaaaacctgttgaaccacatcaacaaaatgaaaggtgttgccaagtcctaccacgaagtcaaaaccgccgccgatgtcgattccaaaaaatccacgttcgttcgccccatcctgtttgaattgaacaacctcaacgaactgcaacgcgaagtcttcggtcccgtcctgcacgtcgtccgctaccgcgccgacgaactcgacaacgtcatcgaccaaatcaacagcaaaggctacgccctgacccacggcgtacacagccgcatcgaaggcacggtacgccacatccgcagccgcatcgaagccggcaacgtttacgtcaaccgcaacatcgtcggcgcagtcgtcggcgtacagcccttcggcggacacggtctgtccggcacaggccccaaagcaggcggttcgttctacctgcaaaaactgacccgcgccggcgaatgggttgccccgaccctgagccaaatcggacaggcggacgaagccgcactcaaacgcctcgaagcactggttcacaaactaccgttcaacgccgaagagaaaaaagccgcagcggccgctttgggacacgcccgcatccgcaccctgcgccgtgccgaaaccgtccttaccggaccgaccggcgagcgcaacagcatctcatggcacgcgcccaaacgcgtttggatacacggcggcagcacggttcaagcctttgccgcactgaccgaacttgccgcctccggcatacaggcagtggtcgaacccgacagccccttggcttcctacactgccgacttggaaggtctgctgctggtcaacggcaaacccgaaaccgccggcatcagccacgttgccgccctgtcgcctttggacagcgcgcgcaaacaggaacttgccgcccacgacggcgcactcatccgcatcctcccttcggaaaacggactcgacatcctgcaagtgtttgaagaaatctcttgcagcgtcaacaccacagccgccggcggcaacgccagcctgatggcggtcgccgactga

SEQ ID NO: 36 - protein, 1201 aa, Neisseria meningitidis (group B)
MFHFAFPAQTALRQAITDAYRRNEIEAVQDMLQRAQMSDEERNAASELARRLVTQVRAGRTKAGGVDALMHEFSLSSEEGIALMCLAEALLRIPDNATRDRLIADKISDGNWKSHLNNSPSLFVNAAAWGLLITGKLTATNDKQMSSALSRLISKGGAPLIRQGVNYAMRLLGKQFVTGQTIEEALQNGKEREKMGYRFSFDMLGEAAYTQADADRYYRDYVEAIHAIGKDAAGQGVYEGNGISVKLSAIHPRYSRTQHGRVMGELLPRLKELFLLGKKYDIGINIDAEEANRLELSLDLMEALVSDPDLAGYKGIGFVVQAYQKRCPFVIDYLIDLARRNNQKLMIRLVKGAYWDSEIKWAQVDGLNGYPTYTRKVHTDISYLACARKLLSAQDAVFPQFATHNAYTLGAIYQMGKGKDFEHQCLHGMGETLYDQVVGPQNLGRRVRVYAPVGTHETLLAYLVRRLLENGANSSFVNQIVDENISIDTLIRSPFDTIAEQGIHLHNALPLPRDLYGKCRLNSQGVDLSNENVLQQLQEQMNKAAAQDFHAASIVNGKARDVGEAQPIKNPADHDDIVGTVSFADAALAQEAVGAAVAAFPEWSATPAAERAACLRRFADLLEQHTPALMMLAVREAGKTLNNAIAEVREAVDFCRYYANEAEHTLPQDAKAVGAIVAISPWNFPLAIFTGEVVSALAAGNTVIAKPAEQTSLIAGYAVSLMHEAGIPTSALQLVLGAGDVGAALTNDARIGGVIFTGSTEVARLINKALAKRGDNPVLIAETGGQNAMIVDSTALAEQVCADVLNSAFDSAGQRCSALRILCVQEDVADRMLDMIKGAMDELVVGKPIQLTTDVGPVIDAEAQQNLLNHINKMKGVAKSYHEVKTAADVDSKKSTFVRPILFELNNLNELQREVFGPVLHVVRYRADELDNVIDQINSKGYALTHGVHSRIEGTVRHIRSRIEAGNVYVNRNIVGAVVGVQPFGGHGLSGTGPKAGGSFYLQKLTRAGEWVAPTLSQIGQADEAALKRLEALVHKLPFNAEEKKAAAAALGHARIRTLRRAETVLTGPTGERNSISWHAPKRVWIHGGSTVQAFAALTELAASGIQAVVEPDSPLASYTADLEGLLLVNGKPETAGISHVAALSPLDSARKQELAAHDGALIRILPSENGLDILQVFEEISCSVNTTAAGGNASLMAVAD

SEQ ID NO: 37 - DNA, 1509 bp, Neisseria meningitidis (group B)
atgaccaccacccccgcaaacgtcctcgcctccgtcgatttgggttccaacagtttccgcctccagatttgcgaaaacaacaacggacaattaaaagtcatcgattcgttcaaacagatggtgcgcttcgccgccggactggacgaacagaaaaatctgagtgccgcttcccaagaacaggctttggactgtctggcaaaattcggcgaacgcctgcgcggcttccgccctgaacaggtacgcgccgtggcaaccaacacattccgcgttgccaaaaacatcgcagatttccttcccaaagccgaagcggcattgggtttccccatcgaaatcatcgccgggcgcgaagaggcgcggctgatttataccggcgtgatccacaccctccccccgggcggcggcaaaatgctggttatcgacatcggcggcggttcgacagaatttgtcatcggctcgacgctgaatcccgacattaccgaaagcctgcccttgggctgcgtaacctacagcctgcgcttcttccaaaacaaaatcaccgccaaagacttccaatctgccatttccgccgcccgcaacgaaatccagcgtatcagcaaaaatatgaggcgcgaaggttgggatttcgccgtcggcacatcgggttcggcaaaatccatccgcgacgtgcttgccgccgaaatgccccaagaggcggacattacctacaaaggcatgcgcgccctcgccgaacgcatcatcgaagccggttcggtcaaaaaagccaaatttgaaaacctgaaaccggaacgcatcgaagtttttgccggcggacttgccgtgatgatggcggcgtttgaggaaatgaaactcgacaggatgaccgtaaccgaagccgccctgcgcgacggcgtgttttacgatttgatcgggcgcggtttaaacgaagatatgcgcggacaaacggttgccgagttccaacaccgctaccacgtcagcctcaatcaggcgaaacgcaccgccgagaccgcgcaaacctttatggacagcctctgccacgctaaaaacgttacagttcaagagcttgccttgtggcaacagtatctcggacgcgccgccgcgctgcacgaaatcggtttggacatcgcccacaccggctatcacaagcattccgcctacatcctcgaaaacgccgatatgccgggtttctcacgcaaagaacagaccatacttgcccaactggtcatcggtcatcgcggcgatatgaaaaaaatgagcggcatcatcggcaccaacgaaatgttgtggtatgccgttttgtccctgcgccttgccgcactgttctgccgttcgcgccaagacctgtctttcccgaaaaatatgcagttgcgcacggatacggaaagctgcggcttcatcctgcgtattgacagggaatggctggaacgccatcccctgattgccgacgcattggaatatgaaagcgtccaatggcaaaaaatcaatatgccgttcaaagtcgaggccgtctga

SEQ ID NO: 38 - protein, 323 aa, Neisseria meningitidis (group B)
MTTTANVASVDGSNSRCNNNGKVDSKMVRAAGDKNSAASADCAKGRRGRVRAVATNTRVAKNADKAAAGAGRARYTGVHTGGGKMVDGGGSTVGSTNDTSGCVTYSRNKTAKDSASAARNRSKNMRRGWDAVGTSGSAKSRDVAAMADTYKGMRAARAGSVKKAKNKRVAGGAVMMAAMKDRMTVTAARDGVYDGRGNDMRGTVAHRYHVSNAKRTATATMDSCHAKNVTVAWYGRAAAHGDAHTGYHKHSAYNADMGSRKTAVGHRGDMKKMSGGTNMWYAVSRAACRSRDSKNMRTDTSCGRDRWRHADAYSVWKNMKVAV

SEQ ID NO: 39 - DNA, 393 bp, Neisseria meningitidis (group B)
atgaacggtaaatactactacggcacaggccgccgcaaaagttcagtggctcgtgtattcctgattaaaggtacaggtcaaatcatcgtaaacggtcgtcccgttgacgaattcttcgcacgggaaaccagccgaatggttgttcgccaacccttggttctgactgaaaacgccgaatctttcgacatcaaagtcaatgttgttggcggcggcgaaaccggccagtccggcgcaatccgccacggcattacccgtgccctgatcgacttcgatgccgcgttgaaacccgccttgtctcaagctggttttgttacccgcgatgcccgcgaagtcgaacgtaaaaaaccgggtctgcgcaaagcacgccgtgcaaaacaattctccaaacgttaa

SEQ ID NO: 40 - protein, 130 aa, Neisseria meningitidis (group B)
MNGKYYYGTGRRKSSVARVFLIKGTGQIIVNGRPVDEFFARETSRMVVRQPLVLTENAESFDIKVNVVGGGETGQSGAIRHGITRALIDFDAALKPALSQAGFVTRDAREVERKKPGLRKARRAKQFSKR

SEQ ID NO: 41 - DNA, 567 bp, Neisseria meningitidis (group B)
atgtccgccctcctccccatcatcaaccgcctgattctgcaaagcccggacagccgctcggaacttgccgcctttgcaggcaaaacactgaccctgaacattgccgggctgaaactggcgggacgcatcacggaagacggtttgctctcggcgggaaacggctttgcagacaccgaaattaccttccgcaacagcgcggtacagaaaatcctccaaggaggcgaacccggggcgggcgacatcgggctcgaaggcgacctcatcctcggcatcgcggtactgtccctgctcggcagcctgcgttcccgcgcatcggacgaattggcacggattttcggcacgcaggcagacatcggcagccgtgccgccgacatcggacacggcatcaaacaaatcggcaggaacatcgccgaacaaatcggcggattttcccgcgaatccgagtccgcaaacatcggcaacgaagcccttgccgactgcctcgacgaaataagcagactgcgcgacggcgtggaacgcctcaacgaacgcctcgaccggctcgaacgcgacatttggatagactaa

SEQ ID NO: 42 - protein, 188 aa, Neisseria meningitidis (group B)
MSALLPIINRLILQSPDSRSELAAFAGKTLTLNIAGLKLAGRITEDGLLSAGNGFADTEITFRNSAVQKILQGGEPGAGDIGLEGDLILGIAVLSLLGSLRSRASDELARIFGTQADIGSRAADIGHGIKQIGRNIAEQIGGFSRESESANIGNEALADCLDEISRLRDGVERLNERLDRLERDIWID

SEQ ID NO: 43 - DNA, 627 bp, Neisseria meningitidis (group B)
atgaacgttaatgttatcaaccatccgctcgtccgccacaaattaaccctgatgagggaggcggattgcagcacctacaaattccggacgcttgccaccgagctggcgcgcctgatggcatacgaggcaagccgtgattttgaaatcgaaaaataccttatcgacggatggtgcggtcagattgaaggcgaccgcatcaagggcaaaacattgaccgtcgttcccatactgcgtgcaggtttgggtatgcttgacggtgtgctcgacctgattccgactgccaaaatcagtgtagtcggactgcagcgcgacgaagaaacgctgaagcctatttcctattttgagaaatttgtggacagtatggacgaacgtccggctttgattatcgatcctatgctggcgacaggcggttcgatggttgccaccatcgaccttttgaaagccaagggctgcaaaaatatcaaggcactggtgctggttgccgcgcccgagggtgtgaaggcggtcaacgacgcgcaccctgacgttacgatttacaccgccgcgctcgacagccacttgaacgagaacggctacatcatccccggcttgggcgatgcgggcgacaagattttcggcacgcgctaa

SEQ ID NO: 44 - protein, 208 aa, Neisseria meningitidis (group B)
MNVNVINHPLVRHKLTLMREADCSTYKFRTLATELARLMAYEASRDFEIEKYLIDGWCGQIEGDRIKGKTLTVVPILRAGLGMLDGVLDLIPTAKISVVGLQRDEETLKPISYFEKFVDSMDERPALIIDPMLATGGSMVATIDLLKAKGCKNIKALVLVAAPEGVKAVNDAHPDVTIYTAALDSHLNENGYIIPGLGDAGDKIFGTR

SEQ ID NO: 45 - DNA, 657 bp, Neisseria meningitidis (group B)
ttggcgtttactttaatgcgtcgcgccatgatacgtaaaatgccctatacggaagatatgcgcccaggcgataccgctaatccttatggtgcgtccaaagcgatggtggaacggatgttaaccgacatccaaaaagccgatccgcgctggagcatgattttgttgcgttatttcaatccgattggcgcgcatgaaagcggcttgattggcgagcagccaaacggcatcccgaataatttgttgccttatatctgccaagtggcggcaggcaaactgccgcaattggcggtatttggcgatgactaccctacccccgacggcacggggatgcgtgactatattcatgtgatggatttggcagaaggccatgtcgcggctatgcaggcaaaaagtaatgtagcaggcacgcatttgctgaacttaggctccggccgcgcttcttcggtgttggaaatcatccgcgcatttgaagcagcttcgggtttgacgattccgtatgaagtcaaaccgcgccgtgccggtgatttggcgtgcttctatgccgacccttcctatacaaaggcgcaaatcggctggcaaacccagcgtgatttaacccaaatgatggaagactcatggcgctgggtgagtaataatccgaatggctacgacgattaa

SEQ ID NO: 46 - protein, 218 aa, Neisseria meningitidis (group B)
MAFTLMRRAMIRKMPYTEDMRPGDTANPYGASKAMVERMLTDIQKADPRWSMILLRYFNPIGAHESGLIGEQPNGIPNNLLPYICQVAAGKLPQLAVFGDDYPTPDGTGMRDYIHVMDLAEGHVAAMQAKSNVAGTHLLNLGSGRASSVLEIIRAFEAASGLTIPYEVKPRRAGDLACFYADPSYTKAQIGWQTQRDLTQMMEDSWRWVSNNPNGYDD

SEQ ID NO: 47 - DNA, 999 bp, Neisseria meningitidis (group B)
atgagcagacccgtacccgccgtattcggcagcgtttttcacagtcaaatgcccgtcctcgcctaccgcgaaggcaaatggcagccgaccgaatggcaatcttcccaagacctctccctcgcaccgggcgcgcacgccctgcactacggcagcgaatgtttcgagggactgaaagccttccgtcaggcagacggcaaaatcgtgctgttccgtccgactgccaatatcgcgcgtatgcggcaaagtgcggacattttgcacctgccgcgccccgaaaccgaagcttatcttgacgcgctaatcaaattggtcaaacgtgccgccgatgaaattcccgatgcgcctgccgccctgtacctgcgtccgaccttaatcggtaccgatcccgttatcggcaaggccggttctccttccgaaaccgccctgctgtatattttggcttcccccgtcggcgactatttcaaagtcggatcgcccgtcaaaattttggtggaaaccgaacacatccgctgcgccccgcatatgggccgcgtcaaatgcggcggcaactacgcttccgccatgcactgggtgctgaaggcgaaagccgaatatggcgcaaatcaagtcctgttctgcccgaacggcgacgtgcaggaaaccggcgcgtccaactttatcctgattaacggcgatgaaatcattaccaaaccgctgaccgacgagtttttgcacggcgtaacccgcgattccgtactgacggttgccaaagatttgggctataccgtcagcgaacgcaatttcacggttgacgaactcaaagctgcggtggaaaacggtgcggaagccattttgaccggtacggcagccgtcatctcgcccgttacttccttcgtcatcggcggcaaagaaatcgaagtgaaaagccaagaacgcggctatgccatccgtaaggcgattaccgacatccagtatggtttggcggaagacaaatacggctggctggttgaagtgtgctga

SEQ ID NO: 48 - protein, 332 aa, Neisseria meningitidis (group B)
MSRPVPAVFGSVFHSQMPVLAYREGKWQPTEWQSSQDLSLAPGAHALHYGSECFEGLKAFRQADGKIVLFRPTANIARMRQSADILHLPRPETEAYLDALIKLVKRAADEIPDAPAALYLRPTLIGTDPVIGKAGSPSETALLYILASPVGDYFKVGSPVKILVETEHIRCAPHMGRVKCGGNYASAMHWVLKAKAEYGANQVLFCPNGDVQETGASNFILINGDEIITKPLTDEFLHGVTRDSVLTVAKDLGYTVSERNFTVDELKAAVENGAEAILTGTAAVISPVTSFVIGGKEIEVKSQERGYAIRKAITDIQYGLAEDKYGWLVEVC

SEQ ID NO: 49 - DNA, 774 bp, Neisseria meningitidis (group B)
atgagtgcgaacatccttgccatcgccaatcagaagggcggtgtgggcaaaacgacgacgacggtaaatttggcggcttcgctggcatcgcgcggcaaacgcgtgctggtggtcgatttggatccgcagggcaatgcgacgacgggcagcggcatcgacaaggcgggtttgcagtccggcgtttatcaggtcttattgggcgatgcggacgtgcagtcggcggcggtacgcagcaaagagggcggatacgctgtgttgggtgcgaaccgcgcgctggccggcgcggaaatcgaactggtgcaggaaatcgcccgggaagtgcgtttgaaaaacgcgctcaaggcagtggaagaagattacgactttatcctgatcgactgcccgccttcgctgacgctgttgacgcttaacgggctggtggcggcgggcggcgtgattgtgccgatgttgtgcgaatattacgcgctggaagggatttccgatttgattgcgaccgtgcgcaaaatccgtcaggcggtcaatcccgatttggacatcacgggcatcgtgcgcacgatgtacgacagccgcagcaggctggttgccgaagtcagcgaacagttgcgcagccatttcggggatttgctttttgaaaccgtcatcccgcgcaatatccgccttgcggaagcgccgagccacggtatgccggtgatggcttacgacgcgcaggcaaagggtaccaaggcgtatcttgccttggcggacgagctggcggcgagggtgtcggggaaatag

SEQ ID NO: 50 - protein, 257 aa, Neisseria meningitidis (group B)
MSANILAIANQKGGVGKTTTTVNLAASLASRGKRVLVVDLDPQGNATTGSGIDKAGLQSGVYQVLLGDADVQSAAVRSKEGGYAVLGANRALAGAEIELVQEIAREVRLKNALKAVEEDYDFILIDCPPSLTLLTLNGLVAAGGVIVPMLCEYYALEGISDLIATVRKIRQAVNPDLDITGIVRTMYDSRSRLVAEVSEQLRSHFGDLLFETVIPRNIRLAEAPSHGMPVMAYDAQAKGTKAYLALADELAARVSGK

SEQ ID NO: 51 - DNA, 1335 bp, Neisseria meningitidis (group B)
atgactgacctgaacaccctgtttgccaacctcaaacaacgcaatcccaatcaggagccgttccatcaggcggttgaagaagtcttcatgagtctcgatccgtttttggcaaaaaatccgaaatacacccagcaaagcctgctggaacgcatcgtcgaacccgaacgcgtcgtgatgttccgcgtaacctggcaggacgataaagggcaagtccaagtcaaccggggctaccgcgtgcaaatgagttccgccatcggtccttacaaaggcggcctgcgcttccatccgaccgtcgatttgggcgtattgaaattcctcgcttttgaacaagtgttcaaaaacgccttgaccaccctgcctatgggcggcggcaaaggcggttccgacttcgaccccaaaggcaaatccgatgccgaagtaatgcgcttctgccaagcctttatgaccgaactctaccgccacatcggcgcggacaccgatgttccggccggcgacatcggcgtaggcgggcgcgaaatcggctacctgttcggacaatacaaaaaaatccgcaacgagttttcttccgtcctgaccggcaaaggtttggaatggggcggcagcctcatccgtcccgaagcgaccggctacggctgcgtctatttcgcccaagcgatgctgcaaacccgcaacgatagttttgaaggcaaacgcgtcctgatttccggctccggcaatgtggcgcaatacgccgccgaaaaagccatccaactgggtgcgaaagtactgaccgtttccgactccaacggcttcgtcctcttccccgacagcggtatgaccgaagcgcaactcgccgccttgatcgaattgaaagaagtccgccgcgaacgcgttgccacctacgccaaagagcaaggtctgcaatactttgaaaaacaaaaaccgtggggcgtcgccgccgaaatcgccctgccctgcgcgacccagaacgaattggacgaagaagccgccaaaaccctgttggcaaacggctgctacgtcgttgccgaaggtgcgaatatgccgtcgactttgggcgcggtcgagcaatttatcaaagccggcatcctctacgccccgggaaaagcctccaatgccggcggcgtggcaacttcaggtttggaaatgagccaaaacgccatccgcctgtcttggactcgtgaagaagtcgaccaacgcctgttcggcatcatgcaaagcatccacgaatcctgtctgaaatacggcaaagtcggcgacacagtaaactacgtcaatggtgcgaacattgccggtttcgtcaaagttgccgatgcgatgctggcgcaaggcttctaa

SEQ ID NO: 52 - protein, 444 aa, Neisseria meningitidis (group B)
MTDLNTLFANLKQRNPNQEPFHQAVEEVFMSLDPFLAKNPKYTQQSLLERIVEPERVVMFRVTWQDDKGQVQVNRGYRVQMSSAIGPYKGGLRFHPTVDLGVLKFLAFEQVFKNALTTLPMGGGKGGSDFDPKGKSDAEVMRFCQAFMTELYRHIGADTDVPAGDIGVGGREIGYLFGQYKKIRNEFSSVLTGKGLEWGGSLIRPEATGYGCVYFAQAMLQTRNDSFEGKRVLISGSGNVAQYAAEKAIQLGAKVLTVSDSNGFVLFPDSGMTEAQLAALIELKEVRRERVATYAKEQGLQYFEKQKPWGVAAEIALPCATQNELDEEAAKTLLANGCYVVAEGANMPSTLGAVEQFIKAGILYAPGKASNAGGVATSGLEMSQNAIRLSWTREEVDQRLFGIMQSIHESCLKYGKVGDTVNYVNGANIAGFVKVADAMLAQGF

SEQ ID NO: 53 - protein, 867 aa, Neisseria meningitidis (group B)
ATGAAAGGCATCATACTGGCAGGCGGCAGCGGCACGCGCCTCTACCCCATCACGCGCGGCGTATCCAAACAGCTCCTGCCCGTGTACGACAAACCGATGATTTATTACCCCTTGTCGGTTTTGATGCTGGCGGGAATCCGCGATATTTTGGTGATTACCGCGCCTGAAGACAACGCCTCTTTCAAACGCCTGCTTGGCGACGGCAGCGATTTCGGCATTTCCATCAGTTATGCCGTGCAACCCAGTCCGGACGGCTTGGCACAGGCATTTATCATCGGCGAAGAATTTATCGGCAACGACAATGTTTGCTTGGTTTTGGGCGACAATATTTTTTACGGTCAGTCGTTTACGCAAACATTGAAACAGGCGGCAGCGCAAACGCACGGCGCAACCGTGTTTGCTTATCAGGTCAAAAACCCCGAACGTTTCGGCGTGGTTGAATTTAACGAAAACTTCCGCGCCGTTTCCATCGAAGAAAAACCGCAACGGCCCAAATCCGATTGGGCGGTAACCGGCTTGTATTTCTACGACAACCGCGCCGTCGAGTTCGCCAAACAGCTCAAACCGTCCGCACGCGGCGAATTGGAAATTACCGACCTCAACCGGATGTATTTGGAAGACGGCTCGCTCTCCGTTCAAATATTGGGACGCGGTTTCGCGTGGCTGGACACCGGCACCCACGAGAGCCTGCACGAAGCCGCTTCATTCGTCCAAACCGTGCAAAATATCCAAAACCTGCACATCGCCTGCCTCGAAGAAATCGCTTGGCGCAACGGTTGGCTTTCCGATGAAAAACTGGAAGAATTGGCGCGCCCGATGGCGAAAAACCAATACGGCCAATATTTGCTGCGCCTGTTGAAAAAATAA

SEQ ID NO: 54 - protein, 288 aa, Neisseria meningitidis (group B)
MKGIILAGGSGTRLYPITRGVSKQLLPVYDKPMIYYPLSVLMLAGIRDILVITAPEDNASFKRLLGDGSDFGISISYAVQPSPDGLAQAFIIGEEFIGNDNVCLVLGDNIFYGQSFTQTLKQAAAQTHGATVFAYQVKNPERFGVVEFNENFRAVSIEEKPQRPKSDWAVTGLYFYDNRAVEFAKQLKPSARGELEITDLNRMYLEDGSLSVQILGRGFAWLDTGTHESLHEAASFVQTVQNIQNLHIACLEEIAWRNGWLSDEKLEELARPMAKNQYGQYLLRLLKK

SEQ ID NO: 55 - DNA, 918 bp, Neisseria meningitidis (group B)
atgaatttgactaaaacacaacgccaactgcacaactttctgaccctcgcccaagaagcaggttcgctgtccaagctcgccaaactctgcggctaccgtacccccgtcgcactctacaaactcaaacaacgccttgaaaagcaggcagaagacccagatgcacgcggcatccgtcccagcctgatggcaaaactcgaaaaacacaccggcaaacccaaaggctggctcgacagaaaacaccgcgaacgcactgtccccgaaaccgccgcagaaagcaccggaactgccgaaacccaaattgccgaaaccgcatctgctgccggctgccgcagcgttaccgtcaaccgcaatacctgcgaaacccaaatcaccgtctccatcaacctcgacggcagcggcaaaagcaggctggataccggcgtacccttcctcgaacacatgatcgatcaaatcgcccgccacggcatgattgacatcgacatcagctgcaaaggcgacctgcacatcgacgaccaccacaccgccgaagacatcggcatcacactcggacaagcaatccggcaggcactcggcgacaaaaaaggcatccgccgttacggacattcctacgtcccgctcgacgaagccctcagccgcgtcgtcatcgacctttccggccgccccggactcgtgtacaacatcgaatttacccgcgcactaatcggacgtttcgatgtcgatttgtttgaagaatttttccacggcatcgtcaaccacagtatgatgaccctgcacatcgacaacctcagcggcaaaaacgcccaccatcaggcggaaaccgtattcaaagccttcgggcgcgccctgcgtatggcagtcgaacacgacccgcgcatggcaggacagaccccctcgaccaaaggcacgctgaccgcataa

SEQ ID NO: 56 - protein, 305 aa, Neisseria meningitidis (group B)
MNLTKTQRQLHNFLTLAQEAGSLSKLAKLCGYRTPVALYKLKQRLEKQAEDPDARGIRPSLMAKLEKHTGKPKGWLDRKHRERTVPETAAESTGTAETQIAETASAAGCRSVTVNRNTCETQITVSINLDGSGKSRLDTGVPFLEHMIDQIARHGMIDIDISCKGDLHIDDHHTAEDIGITLGQAIRQALGDKKGIRRYGHSYVPLDEALSRVVIDLSGRPGLVYNIEFTRALIGRFDVDLFEEFFHGIVNHSMMTLHIDNLSGKNAHHQAETVFKAFGRALRMAVEHDPRMAGQTPSTKGTLTA

(19) United States
(12) Patent Application Publication
(10) Pub. No.: US 2007/0275017 A1
(43) Pub. Date: Nov. 29, 2007

(54) Identification of Antigenically Important Neisseria Antigens by Screening Insertional Mutant Libraries with Antiserum

(75) Inventors: Christoph Marcol Tang, London (GB); Yanwen Li, London (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Imperial Innovations Limited, London (BG)

(21) Appl. No.: 10/584,367

(22) PCT Filed: Dec. 23, 2004
(86) PCT No.: PCT/GB04/05441

§ 371 Date: May 22, 2007

(30) Foreign Application Priority Data
Dec. 23, 2003 (GB) 0330007.6

(51) Int. Cl. G01N 33/554; C07H 21/04; C07K 14/22; C12P 19/34

(52) U.S. Cl. 424/250.1; 435/252.3; 435/7.32; 435/71.2; 435/91.1; 514/12; 514/44; 530/350; 536/23.7

(57) ABSTRACT

A method for identifying a polypeptide of a microorganism which polypeptide is associated with an immune response in an animal which has been subjected to the microorganism, the method comprising the steps of (1) providing a plurality of different mutants of the microorganism; (2) contacting the plurality of mutant microorganisms with antibodies from an animal which has raised an immune response to the microorganism or a part thereof, under conditions whereby if the antibodies bind to the mutant microorganism the mutant microorganism is killed; (3) selecting surviving mutant microorganisms from step (2); (4) identifying the gene containing the mutation in any surviving mutant microorganism; and (5) identifying the polypeptide encoded by the gene. The polypeptide identified or a variant or fragment thereof or a fusion of these is useful in a vaccine. The polypeptide may be a polypeptide comprising the amino acid sequence selected from any one of SEQ ID Nos 2, 4, 6, 8, 10, 12, 14, 16, 18, 25 20, 22, 24, 26, 28, 30, 32, 34, 36, 38, 40, 42, 44, 46, 48, 50, 52, 54, 56; or a fragment or variant thereof or a fusion of such a fragment or variant, and is useful in a vaccine against Neisseria meningitidis.

The present invention relates to vaccines, and in particular to a method of identifying microbial polypeptides which are vaccine candidates.

The listing or discussion of a prior-published document in this specification should not necessarily be taken as an acknowledgement that the document is part of the state of the art or is common general knowledge. The documents listed in the specification are hereby incorporated by reference.

Microbial infections remain a serious risk to human and animal health, particularly in light of the fact that many pathogenic microorganisms, particularly bacteria, are or may become resistant to anti-microbial agents such as antibiotics.

Vaccination provides an alternative approach to combating microbial infections, but it is often difficult to identify suitable iumunogens for use in vaccines which are safe and which are effective against a range of different isolates of a pathogenic microorganism, particular a genetically diverse microorganism. Although it is possible to develop vaccines which use as the immunogen substantially intact microorganisms, such as live attenuated bacteria which typically contain one or mutations in a virulence-determining gene, not all microorganisms are amenable to this approach, and it is not always desirable to adopt this approach for a particular microorganism where safety cannot always be guaranteed. Also, some microorganisms express molecules which mimic host proteins, and these are undesirable in a vaccine.

A particular group of microorganisms for which it is important to develop further vaccines is Neisseria meningitidis which causes meningococcal disease, a life threatening infection which in the Europe, North America, developing countries and elsewhere remains an important cause of childhood mortality despite the introduction of the conjugate serogroup C polysaccharide vaccine. This is because infections caused by serogroup B strains (NmB), which express an α2-8 linked polysialic acid capsule, are still prevalent. The term “serogroup” in relation to N. meningitidis refers to the polysaccharide capsule expressed on the bacterium. The common serogroup in the UK causing disease is B, while in Africa it is A. Meningococcal septicaemia continues to carry a high case fatality rate; and survivors are often left with major psychological and/or physical disability. After a non-specific prodromal illness, meningococcal septicaemia can present as a fulminant disease that is refractory to appropriate anti-microbial therapy and full supportive measures. Therefore, the best approach to combating the public health menace of meningococcal disease is through prophylactic vaccination.

The non-specific early clinical signs and fulminant course of meningococcal infection mean that therapy is often ineffective. Therefore vaccination is considered the most effective strategy to diminish the global disease burden caused by this pathogen (Feavers (2000) ABC of meningococcal diversity. Nature 404, 451-2). Existing vaccines to prevent serogroup A, C, W135, and Y N. meningitidis infections are based on the polysaccharide capsule located on the surface of bacterium (Anderson et al (1994) Safety and immunogenicity of meningococcal A and C polysaccharide conjugate vaccine in adults. Infect imnun. 62, 3391-33955; Leach et al (1997) Induction of immunologic memory in Gambian children by vaccination in infancy with a group A plus group C meningococcal polysaccharide-protein conjugate vaccine. J. Infect Dis. 175, 200-4; Lieberman et al (1996). Safety and immunogenicity of a serogroups A/C Neisseria meningitidis oligosaccharide-protein conjugate vaccine in young children. A randomized controlled trial. J. American Med. Assoc. 275, 1499-1503). Progress toward a vaccine against serogroup B infections has been more difficult as its capsule, a homopolymer of α2-8 linked sialic acid, is a relatively poor immunogen in humans. This is because it shares epitopes expressed on a human cell adhesion molecule, N-CAM1 (Finne et al (1983) Antigenic similarities between brain components and bacteria causing meningitis. Implications for vaccine development and pathogenesis. Lancet 2, 355-357). Indeed, generating immune responses against the serogroup B capsule might actually prove harmful. Thus, there remains a need for new vaccines to prevent serogroup B N. meningitidis infections.

The most validated immunologic correlate of protection against meningococcal disease is the serum bactericidal assay (SBA). The SBA evaluates the ability of antibodies (usually IgG2a subclass) in serum to mediate complement deposition on the bacterial cell surface, assembly of the membrane attack complex, and bacterial lysis. In the SBA, a known number of bacteria are exposed serial dilutions of the sera with a defined complement source. The number of surviving bacteria is determined, and the SBA is defined as the reciprocal of the highest dilution of serum that mediates 50% killing. The SBA is predictive of protection against serogroup C infections, and has been widely used as a surrogate for immunity against NmB infections. Importantly the SBA is a ready marker of immunity for the pre-clinical assessment of vaccines, and provides a suitable endpoint in clinical trials.

Most efforts at NmB vaccine development are directed toward defining effective protein subunits. There has been a major investment in ‘Reverse vaccinology’, in which genome sequences are interrogated for potentially surface expressed proteins which are expressed as heterologous antigens and tested for their ability to generate meaningful responses in animals. However, this approach is limited by 1) the computer algorithms for predicting surface expressed antigens, 2) failure to express many of potential immunogens, and 3) the total reliance on murine immune responses.

The key to a successful vaccine is to define antigen(s) that elicit protection against a broad range of disease isolates irrespective of serogroup or clonal group. An object of the invention is to use a novel genetic screening method (which we have termed Genetic Screening for Immunogens or GSI) to isolate antigens that are conserved across the genetic diversity of microbial strains and this is exemplified in relation to meningococcal strains. This may be done by identifying microbial antigens, such as N. meningitidis antigens, by GSI as described in more detail below; and validated by assessing the function of the immune response elicited by the recombinant antigens and by evaluating the protective efficacy of antigens. Although genetics has been applied in the search for vaccine candidates previously, it has hitherto been difficult to establish high throughput analyses, and has been difficult to differentiate between immunogenic and protective antigens.

A first aspect of the invention provides a method for identifying a polypeptide of a microorganism which polypeptide is associated with an immune response in an animal which has been subjected to the microorganism, the method comprising the steps of
(1) providing a plurality of different mutants of the microorganism;
(2) contacting the plurality of mutant microorganisms with antibodies from an animal which has raised an immune response to the microorganism or a part thereof, under conditions whereby if the antibodies bind to the mutant microorganism the mutant microorganism is killed;
(3) selecting surviving mutant microorganisms from step (2);
(4) identifying the gene containing the mutation in any surviving mutant microorganism; and
(5) identifying the polypeptide encoded by the gene.

The immune response with which the polypeptide is associated is a functionally important one in the sense that it is one that may be associated with combating infection of the animal by the microorganism.

The microorganism may be any microorganism, such as a bacterium or yeast It is preferred if the microorganism is a pathogenic microorganism, and particularly a pathogenic bacterium such as Neisseria meningitidis which causes meningococcal disease, or Neisseria gonorrhoeae which causes gonorrhoea, or Haemophilus influenzae which causes at least one type of influenza and middle ear infection.

By “polypeptide associated with an immune response in an animal which has been subjected to the microorganism” we include any such polypeptide. The method of the invention is able to identify polypeptides of microorganisms which polypeptides are ones for which antibodies have been raised in an animal when the immune system of the animal has been in contact with the polypeptide of the microorganism. Typically, the polypeptide is one which is expressed on the surface of the microorganism. The immune response of the animal is an antibody response, typically an IgG response.

In one embodiment of the invention, the animal has been subjected to the microorganism by way of infection with the microorganism, for example a natural infection. Thus, the animal is typically a host for the microorganism. In another embodiment of the invention, the animal has purposefully been inoculated with the microorganism (whether live or killed) or part thereof. Either way, the animal's immune response has given rise to antibodies directed at the microorganism, some of which are selective for particular polypeptides of the microorganism and which can be used to identify polypeptides by the method of the invention.

It will be appreciated that the term “animal” includes human and in a particularly preferred embodiment of the invention, as discussed in more detail below, the antibodies used in step (2) are ones from a human who is or has been infected with a microorganism or has been immunised with part of the microorganism.

By using antibodies from the animal which has raised an immune response to the microorganism or part thereof, immunologically relevant polypeptides may be identified (in the context of immunogenicity and vaccine design, and particularly polypeptides which are protective).

Thus, it will be appreciated that the polypeptides identified using the method of the invention are antigens or immunogens (these terms being used interchangeably in the specification), typically surface exposed, which are ones that give rise to an immune response in an animal and so are vaccine candidates.

The plurality (or library) of different mutant microorganisms typically is a sufficient large number to give a high chance (typically >95%) of each gene within the microorganism being mutated. The number of mutants required will depend on the number of genes in the genome of the microorganism. When the mutants are random mutants, the number of mutants required to give a high chance of a mutant of each gene within the genome being represented is around 10 times the number of genes. Typically, therefore, the number of mutant microorganisms provided in step (1) is around 10 to 20 times the number of genes in the microorganism.

Typically, a bacterium has from 500 to 5000 genes, and so the number of random mutants used is of the order of 5000 to 100,000. In the case of N. meningitidis a suitable number of random mutants is around 40,000.

The random mutants may be any type of random mutant such as those induced chemically; preferably, the mutants are insertion mutants, such as transposon mutants, since it is straightforward to identify the position of the insertions (e.g. using probes or PCR primers which are selective for the inserted element e.g. transposon), and typically the transposon carries an antibiotic resistance marker which allows selection of the mutants containing the transposon.

Transposons suitable for integration into the genome of Gram negative bacteria include Tn5, Tn10 and derivatives thereof. Transposons suitable for integration into the genome of Gram positive bacteria include Tn916 and derivatives or analogues thereof. Transposons particularly suited for use with Staphylococcus aureus include Tn917 (Cheung et al (1992) Proc. Natl. Acad. Sci. USA 89, 6462-6466) and Tn918 (Albus et al (1991) Infect. Immun. 59, 1008-1014).

It is particularly preferred if the transposons have the properties of the Tn917 derivatives described by Camilli et al (1990) J. Bacteriol. 172, 3738-3744, and are carried by a temperature-sensitive vector such as pE194Ts (Villafane et al (1987) J. Bacteriol. 169, 4822-4829).

For N. meningitidis, Tn10 is a preferred transposon (see Sun et al (2000) Nature Med. 6, 1269-1273), although any transposon and transposase with in vitro activity can be used.

It will be appreciated that although transposons are convenient for insertionally inactivating a gene, any other known method, or method developed in the future may be used. A further convenient method of insertionally inactivating a gene, particularly in certain bacteria such as Streptococcus, is using insertion-duplication mutagenesis such as that described in Morrison et al (1984) J. Bacteriol 159, 870 with respect to S. pneumoniae. The general method may also be applied to other microorganisms, especially bacteria.

For fungi, insertional mutations are created by transformation using DNA fragments or plasmids preferably carrying selectable markers encoding, for example, resistance to hygromycin B or phleomycin (see Smith et al (1994) Infect. Immunol. 62, 5247-5254). Random, single integration of DNA fragments encoding hygromycin B resistance into the genome of filamentous fungi, using restriction enzyme mediated integration (REMI; Schiestl & Petes (1991); Lu et al (1994) Proc. Natl. Acad. Sci. USA 91, 12649-12653) are known.

A simple insertional mutagenesis technique for a fungus is described in Schiestl & Petes (1994) incorporated herein by reference, and include, for example, the use of Ty elements and ribosomal DNA in yeast.

Random integration of the transposon or other DNA sequence allows isolation of a plurality of independently mutated microorganisms wherein a different gene is insertionally inactivated in each mutant.

For some microorganisms, libraries of mutants in which each gene is mutated by a transposon or other insertion element are known. In this case, the plurality of microorganisms may conveniently be produced by pooling one or more representatives of each member of the library. For example, a comprehensive transposon library for Pseudomonas aeruginosa is described in Jacobs et al (2003) Proc. Natl. Acad. Sci. USA 100, 14339-14344.

In step (2) of the method, the plurality of mutant microorganisms is contacted with antibodies from the animal which has raised as immune response to the microorganism or part thereof. The antibodies may be in any suitable form and from any suitable, convenient source from the animal (including human). Typically, the antibodies are present in serum derived from the animal. However, they may be present in other forms, such as a fraction enriched for IgG. It is preferred if the antibodies are IgG antibodies, but other antibody types may be used, such as IgA and IgM. Although it is preferred if the antibodies are present in or derived from serum, the antibodies may be present in or derived from other body fluids such as saliva.

The antibodies are typically from an animal which is or has been infected with the microorganism. One of the advantages of this embodiment of the method is that it makes use of antibodies from an animal which has raised a relevant immune response in attempting to combat the infection with the microorganism, and such antibodies are likely to bind to polypeptides which are useful in vaccines. Thus, preferred antibodies are ones which are from humans who are or who have been infected with the microorganism, or who have been inoculated with an attenuated (e.g. vaccine) strain of the microorganism or who have been vaccinated with a vaccine which comprises a part of the microorganism (such as outer membrane component parts). Typically, the antibodies used in step (2) of the method are from an animal (such as man) which has raised a protective response against the microorganism.

Alternatively, the antibodies are from an animal, such as an experimental animal such as mouse, rabbit, sheep or horse, which has been inoculated with the microorganism and allowed to generate an immune response, preferably a protective immune response. Whether or not a protective response has been raised may be determined by challenging the animal with live bacteria after inoculation. The experimental animals may have been inoculated with a virulent, pathogenic strain of the microorganism, or it may have been inoculated with an avirulent or attenuated strain (whether live or killed).

In a preferred embodiment, the antibodies are raised to a strain of microorganism “heterologous” to the strain used to produce the mutant microorganism. Many pathogenic microorganisms exist in different serogroups or strains, and each serogroup or strain may have polypeptides in common with other serogroups or strains as well as polypeptides which are unique to the serogroup or strains. The advantage of using antibodies raised to one or more heterologous strain(s) is that it increases the chances of identifying a polypeptide which is common to all serogroups of the microorganism (i.e. conserved, common epitopes). Such polypeptides (or fragments or variants or fusions thereof) are more likely to be effective against the range of serogroups of a particular microorganism. Thus, in a particularly preferred embodiment where the microorganism is N. meningitidis, the plurality of mutant microorganisms are derived from a parent serogroup B strain, whereas the antibodies are derived from an animal (such as man) which has raised a response to a serogroup A and/or a serogroup C strain. It will be appreciated that antibodies may be pooled from more than one source. For example, serum from a patient infected with (or convalescing from an infection with) serogroup A strain may be pooled with serum from a patient infected with (or convalescing from an infection with) serogroup C strain. Serum from a patient infected with (or convalescing from an infection with) serogroup B strain may also be pooled. Some microorganisms have, in addition to polypeptide components of their cell wall or cell membrane, polysaccharide components which often are immunogenic. In a further preferred embodiment, it is convenient to use a strain of the microorganism in which some or all of the polysaccharide components have been eliminated as the strain against which antibodies are produced. Thus, many bacteria have a capsule made predominantly of polysaccharide, but typically strains exist in which the capsule is missing. These “capsule minus” strains may conveniently be used to raise antibodies for use in the second step of the method.

In relation to N. meningitidis, the antibodies may conveniently be present in the following serum sources: from mice immunized by the systemic route using heterologous strains of N. meningitidis (i.e. heterologous to the mutant strain used in the selection); from both acute and convalescent human patients infected with N. meningitidis; and from human patients immunized with defined outer membrane vesicles (OMVs) vaccine derived from the serogroup B NmB isolate H44/76. Convalescent sera is preferred since the patient will have raised a significant immune response to the infecting bacteria. In some circumstance, it may be useful to use acute patient serum as a control for the convalescent serum since acute patients may not have raised such a significant immune response. Equivalent sources of antibodies are available with respect to other microorganisms.

Conditions are provided so that when the antibodies bind to the mutant microorganism, the mutant microorganism is killed, whereas when the antibodies do not bind to the mutant microorganism, the mutant microorganism is not killed. In this way, it can be seen that those mutant microorganisms in which a gene encoding a polypeptide which binds to the antibody is mutated so that the antibody no longer binds survive. This provides a very powerful selection for such mutants, and facilitates the identification of the polypeptides which are associated with an immune response in an animal infected with microorganism. As a control, it may be convenient to use wild-type microorganisms which are also killed under the given conditions, or to develop conditions in which all wild type microorganisms are killed.

Conveniently, once the antibodies have been contacted with the mutant microorganism a source of complement is added, such as complement from human, rabbit, mouse, sheep and horse. Conveniently, the complement is from the same source (i.e. species of animal) as the antibody. Antibodies (generally IgG 2a subclass) mediate complement deposition on the surface of the microorganism, assembly of the membrane attack complex and lysis of the microorganism. Complement-mediated killing is independent of the presence of cells from the blood, but requires the presence of serum. Complement-mediated killing may be inactivated by heating the serum.

Preferably, in this embodiment, the microorganism is a bacterium, either Gram positive or Gram negative. Complement mediated killing is described in Walport (2001) N. Engl. J. Med. 344, 1140-1144, and Walport (2001) N. Eng J. Med. 344, 1058-1066.

The complement deposition approach to killing the microorganisms which retain the ability to bind to the antibodies is particularly suited to use with N. meningitidis since the serum bactericidal assay (SBA; see Goldschneider et al (1969) J. Exp. Med. 129, 1307-1326), which is based on the same principle, is used as discussed in the introduction. Of course, in the case of the SBA, the number of surviving bacteria is used to assess the effectiveness of serum in killing bacteria (and using this as a marker of the degree of protection conferred by the strain used to give rise to the antibodies use). As far as the inventors are aware there has never been any suggestion that this method could be adapted to identifying antigens in microorganisms.

As an alternative to using complement to kill the cells to which the antibodies bind, a moiety may be used which binds selectively to the antibodies (which bind the cell) and delivers a cytotoxic agent to the cell. For example, the moiety may be a further antibody which recognizes the antibodies bound to the microorganism and delivers the cytotoxic agent to the cell. Thus, the further antibody may be an anti-human antibody when the antibody which binds to the mutant microorganism is a human antibody. The cytotoxic agent may be directly cytotoxic or it may be indirectly cytotoxic. By indirectly cytotoxic we include an enzyme that is capable of activating a relatively nontoxic compound to a cytotoxic compound. A similar technique has been used to target tumour cells using tumour-selective antibodies and has been called ADEPT (antibody-directed enzyme prodrug therapy; see WO 88/07378; Bagshawe (1987) Br. J. Cancer 56, 531-532; Bagshawe et al (1988) Br. J. Cancer 58, 700-703; and Senter et al (1988) Proc. Natl. Acad. Sci. USA 85, 4842-4846, all of winch are incorporated herein by reference).

Enzyme—prodrug pairs include the following: Alkaline phosphatase useful for converting phosphate-containing prodrugs into free drugs, aryl sulphatase useful for converting sulphate-containing prodrugs into free drugs, cytosine deaminase useful for converting non-toxic 5-fluorocytosine into the anticancer drug 5-fluorouracil, proteases such as Serratia protease, thermolysin, subtilisin, carboxy-peptidases and cathepsins that are useful for converting peptide-containing prodrugs into free drugs, D-alanylcarboxypeptidases, useful for converting prodrugs that contain D-amino acid substituents, carbohydrate-enzymes such as galactosidase and neuraminidase useful for converting glycosylated prodrugs into free drugs, β-lactamase useful for converting drugs derivatized with β-lactams into free drugs and penicillin amidases useful for converting drugs derivatized at their amine nitrogens with phenoxyacetyl or phenylacetyl groups into free drugs.

Other enzymes and prodrugs include hydrolases, amidases, sulphatases, lipases, glucuronidases, phosphatases and carboxypeptidases, and prodrugs be prepared from any of the various classes of anti-tumour compounds for example alkylating agents (nitrogen mustards) including cyclophosphamide, bisulphan, chlorambucil and nitrosoureas; intercalating agents including adriamycin and dactinomycin; spindle poisons such as vinca alkaloids; and anti-metabolites including anti-folates, anti-purines, anti-pyrimidines or hydroxyurea.

Also included are cyanogenic prodrugs such as amygdalin which produce cyanide upon action with a carbohydrate cleaving enzyme.

Mutant microorganisms which survive the conditions, for example those conditions which kill all wild type (or parent) microorganisms (and indeed the majority of mutants), are selected for further study since such mutant microorganisms are likely to be mutated in a gene which encodes a polypeptide which binds to the antibodies (and therefore is involved in an immune response). In one embodiment, and in order to confirm that the mutations in the surviving mutants are responsible for conferring the ability to withstand killing, the mutation of each mutant may backcrossed into the parental strain and the ability of the backcrossed mutant to survive the killing conditions confirmed.

The gene containing the mutation is identified using methods well known in the art. For example, when the mutation is an insertion mutation, it is convenient to sequence from the insertion into the flanking DNA of the microorganism. When a transposon has been used to create the mutant microorganisms, it is convenient to identify the gene containing the transposon mutation by digesting genomic DNA from the individual mutant selected in step (3) with a restriction enzyme which cuts outside the transposon, ligating size-fractionated DNA containing the transposon into a plasmid, and selecting plasmid recombinants on the basis of antibiotic resistance conferred by the transposon and not by the plasmid. The microorganism genomic DNA adjacent to the transposon may be sequenced using two primers which anneal to the terminal regions of the transposon, and two primers which anneal close to the polylinker sequences of the plasmid. The sequences may be subjected to DNA database searches to determine if the transposon has interrupted a known gene. Thus, conveniently, sequence obtained by this method is compared against the sequences present in the publicly available databases such as EMBL and GenBank, or a complete genome sequence, if available.

By “gene” we include not only the regions of DNA that code for a polypeptide but also regulatory regions of DNA such as regions of DNA that regulate transcription, translation and, for some microorganisms, splicing of RNA. Thus, the gene includes promoters, transcription terminators, ribosome-binding sequences and for some organisms introns and splice recognition sites.

Typically, sequence information of the identified gene obtained in step 4 is derived. Conveniently, sequences close to the ends of the transposon are used as the hybridisation site of a sequencing primer. The derived sequence or a DNA restriction fragment adjacent to the inactivated gene itself is used to make a hybridisation probe with which to identify, and isolate from a wild-type organism, the corresponding wild type gene.

It is preferred if the hybridisation probing is done under stringent conditions to ensure that the gene, and not a relative, is obtained, at least when identifying the gene.

The gene may be sequenced using standard methods and the polypeptide encoded by the gene identified, for example by translating the coding sequence of the gene, or the sequence may already be available as part of a sequenced microorganism genome.

As described in more detail in the Example, particular genes identified by the method of the invention are the NBM0341 (TspA), NMB0338, NMB1345, NMB0738, NBM0792 (NadC family), NMB0279, NMB2050, NMB1335 (CreA), NMB2035, NMB1351 (Fmu and Fmv), NMB1574 (IIvC), NMB1298 (rsuA), NMB1856 (LysR family), NMB0119, NMB1705 (rfak), NMB2065 (HemK), NMB0339, NMB0401 (putA), NMB1467 (PPX), NMB2056, NMB0808, NMB0774 (upp), NMA0078, NMB0337 (branched-chain amino acid transferase), NMB0191 (ParA family), NMB1710 (glutamate dehydrogenase (gdhA), NMB0062 (rfbA-1) and NMB1583 (hisB) genes of Neisseria meningitidis. The genome sequence for N. meningitidis is available, for example from The Institute of Genome Research (TIGR); www.tigr.org. Although these genes form part of the genome that has been sequenced, as far as the inventors are aware, they have not been isolated, the polypeptides they encode have not been produced, and there is no indication that the polypeptides they encode may be useful as a component of a vaccine.

Thus, the invention includes the isolated genes as above and in the Examples and variants and fragments and fusions of such variants and fragments, and includes the polypeptides that the genes encode as described above, along with variants and fragment thereof, and fusions of such fragments and variants. Variants, fragments and fusions are described in more detail below. Preferably, the variants, fragments and fusions of the given genes above are ones which encode a polypeptide which gives rise to neutralizing antibodies against N. meningitidis. Similarly, preferably, the variants, fragments and fusions of the polypeptide whose sequence is given above are ones which gives rise to neutralizing antibodies against N. meningitidis. The invention also includes isolated polynucleotides encoding the polypeptides whose sequences are given in the Example (preferably the isolated coding region) or encoding the variants, fragments or fusions. The invention also includes expression vectors comprising such polynucleotides and host cells comprising such polynucleotides and vectors (as is described in more detail below). The polypeptides described in the Examples are antigens identified by the method of the invention.

Molecular biological methods for use in the practice of the method of the invention are well known in the art, for example from Sambrook & Russell (2001) Molecular Cloning, a laboratory manual, third edition, Cold Spring Harbor laboratory Press, Cold Spring Harbor, N.Y., incorporated herein by reference.

It will be appreciated that the invention also includes carrying out steps (1) to (4) of the method (but not necessarily step (5)) so that a gene encoding a polypeptide which is associated with an immune response in an animal which has been subjected to the microorganism is identified. The gene may be cloned and sequenced or may be isolated or synthesised, for example by using the polymerase chain reaction using primers based on its sequence. Variants of the gene may be made, for example by identifying related genes in other microorganisms or in other strains of the microorganism, and cloning, isolating or synthesizing the gene. Typically, variants of the gene are ones which have at least 70% sequence identity, more preferably at least 85% sequence identity, most preferably at least 95% sequence identity with the genes isolated by the method of the invention. Of course, replacements, deletions and insertions may be tolerated. The degree of similarity between one nucleic acid sequence and another can be determined using the GAP program of the University of Wisconsin Computer Group.

Variants of the gene are also ones which hybridise under stringent conditions to the gene. By “stringent” we mean that the gene hybridises to the probe when the gene is immobilised on a membrane and the probe (which, in this case is >200 nucleotides in length) is in solution and the immobilised gene/hybridised probe is washed in 0.1×SSC at 65° C. for 10 min. SSC is 0.15 M NaCl/0.015 M Na citrate.

Fragments of the gene (or the variant gene) may be made which are, for example, 20% or 30% or 40% or 50% or 60% or 70% or 80% or 90% of the total of the gene. Preferred fragments include all or part of the coding sequence. The variant and fragments may be fused to other, unrelated, polynucleotides.

Thus the invention also includes a method for making a polynucleotide the method comprising carrying out steps (1) to (4) of the method of the invention and isolating or synthesising the identified gene or a variant or fragment thereof or a fusion of such gene or variant or fragment. The invention also includes a polynucleotide obtainable or obtained by this method.

Preferably, the polynucleotide encodes a polypeptide which is immunogenic and is reactive with the antibodies from an animal which has been subjected to the microorganism from which the gene was identified.

The invention also includes a method of selecting a microorganism mutated in a gene encoding a polypeptide which is associated with an immune response in an animal which has been subjected to the microorganism. This method comprises carrying out steps (1) to (3) of the method of the invention (whether or not steps (4) and (5) are carried out). The invention also included a mutant microorganism obtainable or obtained by this method which is not able to bind to the antibodies.

Although as discussed above the method of the invention is useful in identifying genes and selecting mutant microorganisms, it is particularly preferred if the method is used to identify polypeptides from a microorganism which are associated with an immune response. Once identified, it is desirable to make an antigen based on the polypeptide.

The antigen may be the polypeptide as encoded by the gene identified, and the sequence of the polypeptide may readily be deduced from the gene sequence. In further embodiments, the antigen may be a fragment of the identified polypeptide or may be a variant of the identified polypeptide or may be a fusion of the polypeptide or fragment or variant.

Fragments of the identified polypeptide may be made which are, for example, 20% or 30% or 40% or 50% or 60% or 70% or 80% or 90% of the total of the polypeptide. Typically, fragments are at least 10, 15, 20, 30, 40, 50, 100 or more amino acids, but less than 500, 400, 300 or 200 amino acids. Variants of the polypeptide may be made. By “variants” we include insertions, deletions and substitutions, either conservative or non-conservative, where such changes do not substantially alter the normal function of the protein. By “conservative substitutions” is intended combinations such as Gly, Ala; Val, Ile, Leu; Asp, Glu; Asn, Gln; Ser, Thr; Lys, Arg; and Phe, Tyr. Such variants may be made using the well known methods of protein engineering and site-directed mutagenesis.

A particular class of variants are those encoded by variant genes as discussed above, for example from related microorganisms or other strains of the microorganism. Typically the variant polypeptides have at least 70% sequence identity, more preferably at least 85% sequence identity, most preferably at least 95% sequence identity with the polypeptide identified using the method of the invention.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res 22, 4673-80). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

The fusions may be fusions with any suitable polypeptide. Typically, the polypeptide is one which is able to enhance the immune response to the polypeptide it is fused to. The fusion partner may be a polypeptide that facilitates purification, for example by constituting a binding site for a moiety that can be immobilised in, for example, an affinity chromatography column. Thus, the fusion partner may comprise oligo-histidine or other amino acids which bind to cobalt or nickel ions. It may also be an epitope for a monoclonal antibody such as a Myc epitope.

The invention also includes, therefore, a method of making an antigen as described above, and antigens obtainable or obtained by the method.

The polynucleotides of the invention may be cloned into vectors, such as expression vectors, as is well known on the art. Such vectors maybe present in host cells, such as bacterial, yeast, mammalian and insect host cells. The antigens of the invention may readily be expressed from polyniucleotides in a suitable host cell, and isolated therefrom for use in a vaccine.

Typical expression systems include the commercially available pET expression vector series and E. Coli host cells such as BL21. The polypeptides expressed may be purified by any method known in the art. Conveniently, the antigen is fused to a fusion partner that binds to an affinity column as discussed above, and the fusion is purified using the affinity column (e.g. such as a nickel or cobalt affinity column).

It will be appreciated that the antigen or a polynucleotide encoding the antigen (such as a DNA molecule) is particularly suited for use as in a vaccine. In that case, the antigen is purified from the host cell it is produced in (or if produced by peptide synthesis purified from any contaminants of the synthesis). Typically the antigen contains less that 5% of contaminating material, preferably less than 2%, 1%, 0.5%, 0.1%, 0.01%, before it is formulated for use in a vaccine. The antigen desirably is substantially pyrogen free. Thus, the invention further includes a vaccine comprising the antigen, and method for making a vaccine comprising combining the antigen with a suitable carrier, such as phosphate buffered saline. Whilst it is possible for an antigen of the invention to be administered alone, it is preferable to present it as a pharmaceutical formulation, together with one or more acceptable carriers. The carrier(s) must be “acceptable” in the sense of being compatible with the antigen of the invention and not deleterious to the recipients thereof. Typically, the carriers will be water or saline which will be sterile and pyrogen free.

The vaccine may also conveniently include an adjuvant. Active immunisation of the patient is preferred. In this approach, one or more antigens are prepared in an immunogenic formulation containing suitable adjuvants and carriers and administered to the patient in known ways. Suitable adjuvants include Freund's complete or incomplete adjuvant, muramyl dipeptide, the “Iscoms” of EP 109 942, EP 180 564 and EP 231 039, aluminium hydroxide, saponin, DEAE-dextran, neutral oils (such as miglyol), vegetable oils (such as arachis oil), liposomes, Pluronic polyols or the Ribi adjuvant system (see, for example GB-A-2 189 141). “Pluronic” is a Registered Trade Mark. The patient to be immunised is a patient requiring to be protected from infection with the microorganism.

The aforementioned antigens of the invention (or polynucleotides encoding such antigens) or a formulation thereof may be administered by any conventional method including oral and parenteral (eg subcutaneous or intramuscular) injection. The treatment may consist of a single dose or a plurality of doses over a period of time.

It will be appreciated that the vaccine of the invention, depending on its antigen component (or polynucleotide), may be useful in the fields of human medicine and veterinary medicine.

Diseases caused by microorganisms are known in many animals, such as domestic animals. The vaccines of the invention, when containing an appropriate antigen or polynucleotide encoding an antigen, are useful in man but also in, for example, cows, sheep, pigs, horses, dogs and cats, and in poultry such as chickens, turkeys, ducks and geese.

Thus, the invention also includes a method of vaccinating an individual against a microorganism, the method comprising administering to the individual an antigen (or polynucleotide encoding an antigen) or vaccine as described above. The invention also includes the use of the antigen (or polynucleotide encoding an antigen) as described above in the manufacture of a vaccine for vaccinating an individual.

The antigen of the invention may be used as the sole antigen in a vaccine or it may be used in combination with other antigens whether directed at the same or different disease microorganisms. In relation to N. meningitidis, the antigen obtained which is reactive against NmB may be combined with components used in vaccines for the A and/or C serogroups. It may also conveniently be combined antigenic components which provide protection against Haemophilus and/or Streptococcus pneumoniae. The additional antigenic components may be polypeptides or they may be other antigenic components such as a polysaccharide. Polysaccharides may also be used to enhance the immune response (see, for example, Makela et al (2002) Expert. Rev. Vaccines 1, 399-410).

It is particularly preferred in the above vaccines and methods of vaccination if the antigen is the polypeptide encoded by any of the genes as described above (and in the Examples), such as the NMB0338 gene, or a variant or fragment or fusion as described above (or a polynucleotide encoding said antigen), and that the disease to be vaccinated against is Neisseria meningitidis infection (meningococcal disease).

The invention will now be described in greater detail by reference to the following non-limiting Examples and Figure wherein:

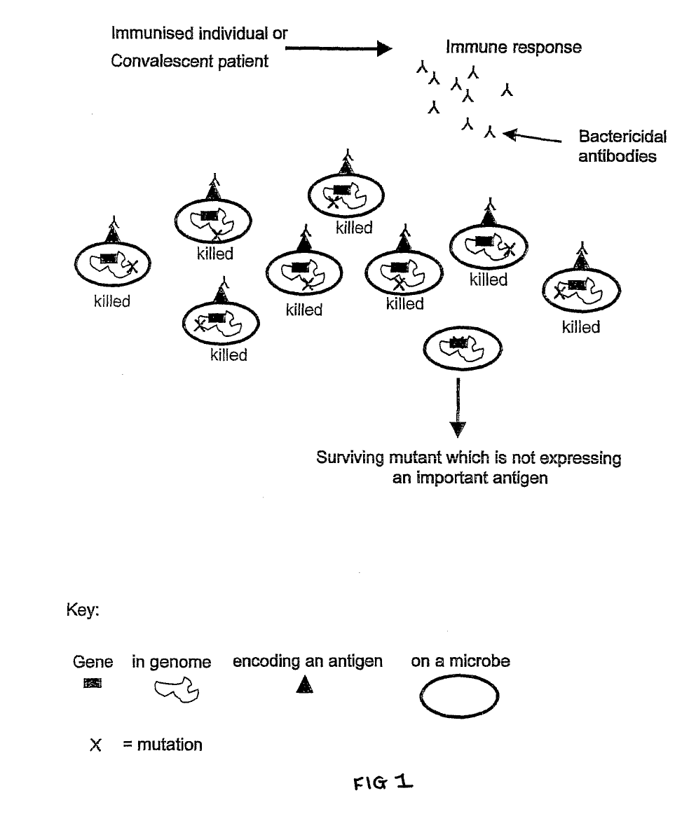

FIG. 1 is a schematic representation of a preferred embodiment of the method of the invention

### EXAMPLE 1

### Genetic Screening for Immunogens (GSI) in N. meningitidis

The application of GSI in this example involves screening libraries of insertional mutants of N. meningitidis for strains which are less susceptible to killing by bactericidal antibodies.

We have demonstrated the effectiveness of GSI by screening a library of mutants of the sequenced NmB isolate, MC58, with sera raised in mice against a capsule minus of the same strain. A total of 40,000 mutants was analysed with sera raised in mice by intraperitoneal immunisation with the homologous strain; the SBA of this sera is around 2,000 against the wild-type strain. Surviving mutants were detected when the library was exposed to serum at a 1:560 dilution (which kills all wild-type bacteria). To establish whether the transposon insertion in the surviving mutants was responsible for the ability to withstand killing, the mutations were backcrossed into the parental strain, and the backcrossed mutants were confirmed as being more resistant to killing than the wild-type in the SBA. The sequence of the gene affected by the transposon was examined by isolating the transposon insertion site by marker rescue. We found that two of the genes affected were TspA and NMB0338. TspA is a surface antigen which elicits strong CD4+ T cell responses and is recognized by sera from patients (Kizil et al (1999) Infect Immun. 67, 3533-41). NMB0338 is a gene of previously unlkown function which encodes a polypeptide that is predicted to contain two transmembrane domains, and is located at the cell surface. The amino acid sequence encoded by NMB0338 is:

MERNGVFGKIVGNRILRMSSEHAAASYPKPCKSFKLAQSWFRVRSCLGGV
FIYGANMKLIYTVIKIIILLLFLLLAVINTDAVTFSYLPGQKFDLPLIVV
LFGAFVVGIIFGMFALFGRLLSLRGENGRLRAEVKKNARLTGKELTAPPA
QNAPESTKQP

There are several practical advantages of using NmB for GSI aside from the public health imperative: a) the bacterium is genetically tractable; b) killing of the bacterium by effector immune mechanism is straightforward to assay; c) the genome sequences are available for three isolates of different serogroups and clonal lineages (IV-A, ET-5, and ET-37 for serogroups A, B, and C, respectively); and d) well-characterised clinical resources are available for this work.

GSI has two potential limitations. First, targets of bactericidal antibodies may be essential. This is unlikely as all known targets of bactericidal antibodies in NmB are non-essential, and no currently licensed bacterial vaccine targets an essential gene product. Second, sera will contain antibodies to multiple antigens, and, loss of a single antigen may not affect the survival of mutants. We have already shown that even during selection with sera raised against the homologus strain, relevant antigens were still identified using appropriate dilutions of sera.

The major advantages of GSI are that 1) the high throughput steps do not involve technically demanding or costly procedures (such as protein expression/purification and immunisation), and 2) human samples can be used in the assay rather than relying solely on animal data. GSI will rapidly pinpoint the subset of surface proteins that elicit bactericidal activity, allowing more detailed analysis of a smaller number of candidates.

1. Identification of Targets of Bactericidal Antibodies Using GSI

Murine sera raised against heterologous strains, and human sera, are used to identify cross-reactive antigens. The sera are obtained from:
i) mice immunised by the systemic route with heterologous strains: the strains will be selected and/or constructed to avoid isolates with the same immunotype and sub-serotype.
ii) acute and convalescent sera from patients infected with known isolates of N. Meningitidis (provided by Dr R. Wall, Northwick Park)
iii) pre- and post-immunisation samples (provided by the Meningococcal Reference Laboratory) from volunteers receiving defined outer membrane vesicle (OMVs) vaccines derived from the NmB isolate, H44/76.

Each of these sources of sera has specific advantages and disadvantages.

| Serum | | |
|---|---|---|
| source | Advantages | Disadvantages |
| Murine | 1) Defined antigenic exposure. | 1) Animal source of |
| | 2) Use of genetically modified | material |
| | strains to generate immune | |
| | response. | |
| | 3) Naïve samples available | |
| | 4) Examine individuals | |
| | responses | |
| Patient | 1) Human material | 1) Background immunity |
| sera | 2) Known strain exposure | 2) Limited material |
| | 3) Acute and convalescent | |
| | sera available | |
| Sera | 1) Human material | 1) Background immunity |
| following | 2) Defined antigenic | 2) Limited material |
| immunisa- | exposure | |
| tion with | 3) Pre and post immunisation | |
| H4476 | sera available | |
| OMVs | 4) Examine individuals | |
| | responses | |

a) Sera from animals immunised with heterologous strains (ie the sequenced serogroup A or C strains) are used in GSI to select the library of MC58 mutants. We have shown that immunisation with live, attenuated NmB elicits cross-reactive bactericidal antibody responses against serogroup A and C strains. The antigen absent in mutants with enhanced survival in the face of human sera are identified by marker rescue of the disrupted gene.

b) Mutations are identified that confer resistance against killing by heterologous sera, and it is determined whether the gene product is also a target for killing of the sequenced, serogroup A and C strains, Z2491 and FAM18 respectively. The genome databases are inspected for homologues of the genes. If a homologue is present, the transposon insertion is amplified from the MC58 mutant and introduced into the serogroup A and C strains by transformation. The relative survival of the mutant and wild-type strain of each serogroup are compared. Thus, GSI can quickly give information whether the targets of bactericidal activity are conserved and accessible in diverse strains of N. meningitidis, irrespective of serogroup, immunotype and subserotype.

c) Mutants with enhanced survival against sera raised in mice are tested using human sera from either convalescent patients or vaccinees receiving heterologous OMV vaccines (derived from H44/76). This addresses the important question of whether the targets are capable of eliciting bactericidal antibodies in human. With other vaccine approaches, this information is only gained at the late, expensive stage of clinical trials that requires GMP manufacture of vaccine candidates.

The advantages are that GSI is a high-throughput analysis performed using simple, available techniques. Antigens which elicit bactericidal antibodies in humans and which mediate killing of multiple strains can be identified rapidly as GSI is flexible with respect to the bacterial strain and sera used. Mutants selected using human sera are analysed in the same way as those selected by murine sera.

2. Assessment of the Antibody Response of Recombinant GSI Antigens

Proteins which are targets of bactericidal antibodies that are recognised by sera from convalescent patients and vaccines are expressed in E. coli using commercially available vectors. The corresponding open reading frames are amplified by PCR from MC58, and ligated into vectors such as pCR Topo CT or pBAD/His, to allow protein expression under the control of a T7 or arabinose-inducible promoter, respectively. Purification of the recombinant proteins from total cellular protein is performed via the His Tag fused to the C terminus of the protein on a Nickel or Cobalt column.

Adult New Zealand White rabbits are immunized on two occasions separated by four weeks by subcutaneous injection with 25 μg of purified protein with Freund's incomplete adjuvant. Sera from animals will be checked prior to immunisation for pre-existing anti-Nm antibodies by whole cell ELISA. Animals which have an initial serum titre of <1:2 are used for immunisation experiments. Post-immunisation serum are obtained two weeks after the second immunisation. To confirm that specific antibodies have been raised, pre- and post-immunisation serum is tested by i) Western analysis against the purified protein and ii) ELISA using cells from the wild-type and the corresponding mutant (generated by GSI).

SBAs will be performed against MC58 (the homologous strain), and the sequenced serogroup A and C strains with the rabbit immune serum. The assay will be performed in triplicate on at least two occasions. SBAs of >8 will be considered significant. The results provide evidence of whether the protein candidates can elicit bactericidal antibodies as recombinant proteins.

3. Establishing the Protective Efficacy of GSI Antigens

All the candidates are tested for their ability to protect animals against live bacterial challenge as this allows any aspect of immunity (cellular or humoral) to be assessed in a single assay. We have established a model of active immunisation and protection against live bacterial infection. In this model, adult mice are immunised on days 0 and 21, and on day 28 receive live bacterial challenge of 10⁶ or 10⁷ CFU of MC58 intraperitoneally in iron dextran (as the supplemental iron source). The model is similar to that described for evaluation of the protective efficacy of immunisation with Tbps Danve et al (1993) Vaccine 11, 1214-1220. Non-immunised animals develop bacteraemia within 4 hours of infection, and show signs of systemic illness by 24 hours. We have already been able to demonstrate the protective efficacy of both attenuated Nm strains and a protein antigen against live meningococcal challenge; PorA is an outer membrane protein that elicits bactericidal antibodies, but which is not a lead vaccine candidate because of extensive antigenic variation (Bart et al (1999) Infect Immun. 67, 3832-3846.

Six week old, BALB/c mice (group size, 35 animals) receive 25 μg of recombinant protein with Freund's incomplete adjuvant subcutaneously on days day 0 and 21, then are challenged with 10⁶ (15 animals) or 10⁷ (15 animals) CFU of MC58 intraperitoneally on day 28. Two challenge doses are used to examine the vaccine efficacy at a high and low challenge dose; sera are obtained on day 28 from the remaining five animals in each group, and from five animals before the first immunisation and stored at −70° C. for further immunological assays. Animals in control groups receive either i) adjuvant alone, ii) recombinant refolded PorA, and iii) a live, attenuated Nm strain. To reduce the overall number of animals in control groups, sets of five candidates will be tested at one time (number of groups=5 candidates+3 controls). Survival of animals in the groups is compared by Mann Whitney U Test. With group sizes of 15 mice/dose, the experiments are powered to show a 25% difference in survival between groups.

For vaccines which show significant protection against challenge, a repeat experiment is performed to confirm the finding. Furthermore, to establish that vaccination with a candidate also elicits protection against bacteraemia, levels of bacteraemia are determined during the second experiment; blood is sampled at 22 hr post-infection in immunised and un-immunised animals (bacteraemia is maximal at this time). The results are analysed using a two-tailed Student-T test to determine if there is a significant reduction in bacteraemia in vaccinated animals.

Further Materials and Methods Used

Mutagenesis of Neisseria meningitidis

For work with Neisseria meningitidis, mutants were constructed by in vitro mutagenesis. Genomic DNA from N. meningitidis, was subjected to mutagenesis with a Tn5 derivative containing a marker encoding resistance to kanamycin, and an origin of replication which is functional in E. coli. These elements are bound by composite Tn5 ends. Transposition reactions were carried out with a hyperactive variant of Tn5 and the DNA repaired with T4 DNA polymerase and ligase in the presence of ATP and nucleotides. The repaired DNA was used to transform N. meningitidis to kanamycin resistance. Southern analysis confirmed that each mutant contained a single insertion of the transposon only.

Serum Bactericidal Assays (SBAs)

Bacteria were grown overnight on solid media (brain heart infusion media with Levanthals supplement) and then re-streaked to solid media for four hours on the morning of experiments. After this time, bacteria were harvested into phosphate buffered saline and enumerated. SBAs were performed in a 1 ml volume, containing a complement source (baby rabbit or human) and approximately 10⁵ colony forming units. The bacteria were collected at the end of the incubation and plated to solid media to recover surviving bacteria.

Isolating the Transposon Insertion Sites

Genomic DNA will be recovered from mutants of interest by standard methods and digested with PvuII, EcoRV, and DraI for three hours, then purified by phenol extraction. The DNA will then be self-ligated in a 100 microlitre volume overnight at 16° C. in the presence of T4 DNA ligase, precipitated, then used to transform E. coli to kanamycin resistance by electroporation.

### EXAMPLE 2

### Further Screening and Results Thereof

GSI has been used to screen a library of approximately 40,000 insertional mutants of MC58. The library was constructed by in vitro Tn5 mutagenesis, using a transposon harbouring the origin of replication from pACYC184.

MC58 was chosen as it is a serogroup B isolate of N. meningitidis, and the complete genome sequence of this strain is known.

The library is always screened in parallel with the wild-type strain as a control, and the number of colonies recovered from the library and the wild-type is shown.

Selection with Murine Sera

Initially the library was analysed using sera from animals immunised with the attenuated strain YH102. Adult mice (Balb/C) received 108 colony forming units intra-peritoneally on three occasions, and sera was collected 10 days after the final immunisation,

The screen identified several mutants with enhanced resistance to serum killing: This was confirmed by isolating individual mutants, reconstructing the mutation in the original genetic background, and re-testing the individual mutants for their susceptibility to complement mediated lysis against the wild-tye. The transposon insertions are in the following gene:

NMB0341 (TspA) DNA sequence
ATGCCCGCCGGCCGACTGCCCCGCCGATGCCCGATGATGACGAAATTTAC
AGACTGTACGCGGTCAAACCGTATTCAGCCCCCAACGCACAGGGGATACA
TCTTGAAAAACAACAGACAAATCAAACTGATTGCCGCCTCCGTCGCAGTT
GCCGCATCCTTTCAGGCACATGCTGGACTGGGCGGACTGAATATCCAGTC
CAACCTTGACGAACCCTTTTCCGGCAGCATTACCGTAACCGGCGAAGAAG
CCAAAGCCCTGCTAGGCGGCGGCAGCGTTACCGTTTCCGAAAAAGGCCTG
ACCGCCAAAGTCCACAAGTTGGGCGACAAAGCCGTCATTGCCGTTTCTTC
CGAACAGGCAGTCCGCGATCCCGTCCTGGTGTTCCGCATCGGCGCAGGCG
CACAGGTACGCGAATACACCGCCATCCTCCATCCTGTCGGCTACTCGCCC
AAAACCAAATCTGCACTTTCAGACGGCAAGACACACCGCAAAACCGCTCC
GACAGCAGAGTCCCAAGAAAATCAAAACGCCAAAGCCCTCCGCAAAACCG
ATAAAAAAGACAGCGCGAACGCAGCCGTCAAACCGGCATACAACGGCAAA
ACCCATACCGTCCGCAAAGGCGAAACGGTCAAACAGATTGCCGCCGCCAT
CCGCCCGAAACACCTGACGCTCGAACAGGTTGCCGATGCGCTGCTGAAGG
CAAACCCAAATGTTTCCGCACACGGCAGACTGCGTGCGGGCAGCGTGCTT
CACATTCCGAATCTGAACAGGATCAAAGCGGAACAACCCAAACCGCAAAC
GGCGAAACCCAAAGCCGAAACCGCATCCATGCCGTCCGAACCGTCCAAAC
AGGCAACGGTAGAGAAACCGGTTGAAAAACCTGAAGCAAAAGTTGCCGCG
CCCGAAGCAAAAGCGGAAAAACCGGCCGTTCGACCCGAACCTGTACCCGC
TGCAAATACTGCCGCATCGGAAACCGCTGCCGAATCCGCCCCCCAAGAAG
CCGCCGCTTCTGCCATCGACACGCCGACCGACGAAACCGGTAACGCCGTT
TCCGAACCTGTCGAACAGGTTTCTGCCGAAGAAGAAACCGAAAGCGGACT
GTTTGACGGTCTGTTCGGCGGTTCGTACACCTTGCTGCTTGCCGGCGGAG
GCGCGGCATTAATCGCCCTGCTGCTGCTTTTGCGCCTTGCCCAATCCAAA
CGCGCGCGCCGTACCGAAGAATCCGTCCCTGAGGAAGAGCCTGACCTTGA
CGACGCGGCAGACGACGGCATAGAAATCACCTTTGCCGAAGTCGAAACTC
CGGCAACGCCCGAACCCGCTCCGAAAAACGATGTAAACGACACACTTGCC
TTAGATGGGGAATCTGAAGAGAAGTTATCGGCAAAACAAACGTTCGATGT
CGAAACCGATACGCCTTCCAACCGCATCGACTTGGATTTCGACAGCCTGG
CAGCCGCGCAAAACGGCATTTTATCCGGCGCACTTACGCAGGATGAAGAA
ACCCAAAAACGCGCGGATGCCGATTCGAACGCCATCGAATCCACAGACAG
CGTGTACGAGCCCGAGACCTTCAACCCGTACAACCCTGTCGAAATCGTCA
TCGACACGCCCGAACCGGAATCTGTCGCCGAAACTGCCGAAAACAAACCG
GAAACCGTCGATACCGATTTCTCCGACAACCTGCCCTCAAACAACCATAT
CGGCACAGAAGAAACAGCTTCCGCAAAACCTGCCTCACCCTCCGGACTGG
CAGGCTTCCTGAAGGCTTCCTCGCCCGAAACCATCTTGGAAAAAACAGTT
GCCGAAGTCCAACACCGGAAAGAGTTGCACGATTTCCTGAAAGTGTACGA
AACCGATGCCGTCGCGGAAACTGCGCCTGAAACGCCCGATTTCAACGCCG
CCGCAGACGATTTGTCCGCATTGCTTCAACCTGCCGAAGCACCGTCCGTT
GAGGAAAATATAACGGAAACCGTTGCCGAAACACCCGACTTCAACGCCAC
CGCAGACGATTTGTCCGCATTACTTCAACCTTCTAAAGTACCTGCCGTTG
AGGAAAATGCAGCGGAAACCGTTGCCGATGATTTGTCCGCACTGTTGCAA
CCTGCTGAAGCACCGGCCGTTGAGGAAAATGTAACGGAAACCGTTGCCGA
AACACCCGATTTCAACGCCACCGCAGACGATTTGTCCGCATTACTTCAAC
CTTCTGAAGCACCTGCCGTTGAGGAAAATGCAGCGGAAACCGTTGCCGAT
GATTTGTCCGCACTGTTGCAACCTGCTGAAGCACCGGCCGTTGAGGAAAA
TGCAGCGGAAATCACTTTGGAAACGCCTGATTCCAACACCTCTGAGGCAG
ACGCTTTGCCCGACTTCCTGAAAGACGGCGAGGAGGAAACGGTAGATTGG
AGCATCTACCTCTCGGAAGAAAATATCCCAAATAATGCAGATACCAGTTT
CCCTTCGGAATCTGTAGGTTCTGACGCGCCTTCCGAAGCGAAATACGACC
TTGCCGAAATGTATCTCGAAATCGGCGACCGCGATGCCGCTGCCGAGACA
GTGCAGAAATTGCTGGAAGAAGCGGAAGGCGACGTACTCAAACGTGCCCA
AGCATTGGCGCAGGAATTGGGTATTTGA
NBM0341 Protein sequence
MPAGRLPRRCPMMTKFTDCTRSNRIQPPTHRGYTLKNNRQTKLIAASVAV
AASFQAHAGLGGLNTQSNLDEPFSGSITVTGEEAKALLGGGSVTVSEKGL
TAKVHKLGDKAVTAVSSEQAVRDPVLVFRTGAGAQVREYTAILDPVGYSP
KTKSALSDGKTHRKTAPTAESQENQNAKALRKTDKKDSANAAVKPAYNGK
THTVRKGETVKQIAAATRPKHLTLEQVADALLKANPNVSAHGRLRAGSVL
HIPNLNRIKAEQPKPQTAKPKAETASMPSEPSKQATVEKPVEKPEAKVAA
PEAKAEKPAVRPEPVPAANTAASETAAESAPQEAAASAIDTPTDETGNAV
SEPVEQVSAEEETESGLFDGLFGGSYTLLLAGGGAALIALLLLLRLAQSK
RARRTEESVPEEEPDLDDAADDGTEITFAEVETRATPEPAPKNDVNDTLA
LDGESEEELSAKQTFDVETDTPSNRIDLDFDSLAAAQNGTLSGALTQDEE
TQKRADADWNAIESTDSVYEPETFNPYNPVEIVIDTPEPESVAQTAENKP
ETVDTDFSDNLPSNNHIGTEETASAKPASPSGLAGFLKASSPETILEKTV
AEVQTPEELHDFLKVYETDAVAETAPETPDFNAAADDLSALLQPAEAPSV
EENITETVAETPDFNATADDLSALLQPSKVPAVEENAAETVADDLSALLQ
PAEAPAVEENVTETVAETPDFNATADDLSALLQPSEAPAVEENAAETVAD
DLSALLQPAEAPAVEENAAEITLETPDSNTSEADALPDFLKDGEEETVDW
SIYLSEENIPNNADTSFPSESVGSDAPSEAKYDLAEMYLEIGDRDAAAET
VQKLLEEAEGDVLKRAQALAQELGI
NMB0338 DNA sequence
ATGGAAAGGAACGGTGTATTTGGTAAAATTGTCGGCAATCGCATACTCCG
TATGTCGTCCGAACACGCTGCCGCATCCTATCCGAAACCGTGCAAATCGT
TTAAACTAGCGCAATCTTGGTTCAGAGTGCGAAGCTGTCTGGGCGGCGTT
TTTATTTACGGAGCAAACATGAAACTTATCTATACCGTCATCAAAATCAT
TATCCTGCTGCTCTTCCTGCTGCTTGCCGTCATTAATACGGATGCCGTTA
CCTTTTCCTACCTGCCGGGGCAAAAATTCGATTTGCCGCTGATTGTCGTA
TTGTTCGGCGCATTTGTAGTCGGTATTATTTTTGGAATGTTTGCCTTGTT
CGGACGGTTGTTGTCGTTACGTGGCGAGAACGGCAGGTTGCGTGCCGAAG
TAAAGAAAAATGCGCGTTTGACGGGGAAGGAGCTGACCGCACCACCGGCG
CAAAATGCGCCCGAATCTACCAAACAGCCTTAA
NMB0338 Protein sequence
MERNGVFGKIVGNRILRMSSEHAAASYPKPCKSFKLAQSWFRVRSCLGGV
FIYGANMKLIYTVTKIIILLLFLLLAVTNTDAVTFSYLPGQKFDLPLIVV
LFGAFVVGIIFGMFALFGRLLSLRGENGRLRAEVKKNARLTGKELTAPPA
QNAPESTKQP

Analysis of the polypeptide indicates that it is predicted to have two membrane spanning domains, from residues 54 to 70 and 88 to 107. Thus, fragments from the regions 1 to 53, and 108 to the end (C-terminal) may be particularly useful as immunogens.

NMB1345 DNA sequence
ATGAAAAAACCTTTGATTTCGGTTGCGGCAGCATTGCTCGGCGTTGCTTT
GGGCACGCCTTATTATTTGGGTGTCAAAGCCGAAGAAAGCTTGACGCAGC
AGCAAAAAATATTGCAGGAAACGGGCTTCTTGACCGTCGAATCGCACCAA
TATGAGCGCGGCTGGTTTACCTCTATGGAAACGACGGTCATCCCTCTGAA
ACCCGAGTTGCTGAATAATGCCCGAAAATACCTGCCGGATAACCTGAAAA
CAGTGTTGGAACAGCCGGTTACGCTGGTTAACCATATCACGCACGGCCCT
TTCGCCGGCGGATTCGGCACGCAGGCGTACATTGAAACCGAGTTCAAATA
CGCGCCTGAAACGGAAAAAGTTCTGGAACGCTTTTTTGGAAAACAAGTCC
CGGCTTCCCTTGCCAATACCGTTTATTTTAACGGCAGCGGTAAAATGGAA
GTCAGTGTTCCCGCCTTCGATTATGAAGAGCTGTCGGGCATCAGGCTGCA
CTGGGAAGGCCTGACGGGAGAAACGATTTATCAAAAAGGTTTCAAAAGCT
ACCGGAACGGCTATGATGCCCCCTTGTTTAAAATCAAGCTGGCAGACAAA
GGCGATGCCGCGTTTGAAAAAGTGCATTTCGATTCGCAAACTTCAGACGG
CATCAATCCGCTTGCTTTGGGCAGCAGCAATCTGACCTTGGAAAAATTCT
CCCTAGAATGGAAAGAGGGTGTCGATTACAACGTCAAGTTAAACGAACTG
GTCAATCTTGTTACCGATTTGCAGATTGGCGCGTTTATCAATCCCAACGG
CAGCATCGCACCTTCCAAAATCGAAGTCGGCAAACTGGCTTTTTCAACCA
AGACCGGGGAATCAGGCGCGTTTATCAACAGTGAAGGGCAGTTCCGTTTC
GATACACTGGTGTACGGCGATGAAAAATACGGCCCGCTGGACATCCATAT
CGCTGCCGAACACCTCGATGCTTCTGCCTTAACCGTATTGAAACGCAAGT
TTGCACAAATTTCCGCCAAAAAAATGACCGAGGAACAAATCCGCAATGAT
TTGATTGCCGCCGTCAAAGGAGAGGCTTCCGGACTGTTCACCAACAATCC
CGTATTGGACATTAAAACTTTCCGATTCACGCTGCCATCGGGAAAAATCG
ATGTGGGCGGAAAAATCATGTTTAAAGACATGAAGAAGGAAGATTTGAAT
CAATTGGGTTTGATGCTGAAGAAAACCGAAGCCGACATCAGAATGAGTAT
TCCCCAAAAAATGCTGGAAGACTTGGCGGTCAGTCAAGCAGGCAATATTT
TCAGCGTCAATGCCGAAGATGAGGCGGAAGGCAGGGCAAGTCTTGACGAG
ATCAACGAGACCTTGCGCCTGATGGTGGACAGTACGGTTCAGAGTATGGC
AAGGGAAAAATATCTGACTTTGAACGGCGACCAGATTGATACTGCCATTT
CTCTGAAAAACAATCAGTTGAAATTGAACGGTAAAACGTTGCAAAACGAA
CCGGAGCCGGATTTTGATGAAGGCGGTATGGTTTCAGAGCCGCAGCAGTA
A
NMB1345 Protein sequence
MKKPLISVAAALLGVALGTPYYLGVKAEESLTQQQKTLQETGFLTVESHQ
YERGWFTSMETTVIRLKPELLNNARKYLPDNLKTVLEQPVTLVNHITHGP
FAGGFGTQAYTETEFKYAPETEKVLERFFGKQVPASLANTVYFNGSGKME
VSVPAFDYEELSGIRLHWEGLTGETVYQKGFKSYRNGYDAPLFKIKLADK
GDAAFEKVHFDSETSDGINPLALGSSNLTLEKFSLEWKEGVDYNVKLNEL
VNLVTDLQIGAFTNPNGSIAPSKTEVGKLAFSTKTGESGAFINSEGQFRF
DTLVYGDEKYGPLDIHIAAEHLDASALTVLKRKFAQISAKKMTEEQIRND
LTAAVKGEASGLFTNNPVLDTKTFRFTLPSGKTDVGGKTMFKDMKKEDLN
QLGLMLKKTEADIRMSIPQKMLEDLAVSQAGNIFSVNAEDEAEGRASLDD
INETLRLMVDSTVQSMAREKYLTLNGDQIDTAISLKNNQLKLNGKTLQNE
PEPDFDEGGMVSEPQQ

Selection with Vaccinees Sera

Sera from the Meningococcal Reference Laboratory in Manchester has been made available to us. This sera has come from a clinical trial of OMV immunisation of volunteers.

Mutants Selected by Vaccinee C1 Sera (Screened Once)

The following sequences were isolated

NMB0338 (as above)

NMB0738 DNA sequence
ATGAAGATCGTCCTGATTAGCGGCCTGTCCGGTTCGGGCAAGTCCCTCGC
ACTGCGCCAAATGGAAGATTCGGGTTATTTCTGCGTGGACAATTTGCCTT
TGGAAATGTTGCCCGCGCTGGTGTCGTATCATATCGAACGTGCGGACGAA
ACCGAATTGGCGGTCAGCGTCGATGTGCGTTCCGGCATTGACATCGGACA
GGCGCGGGAACAGATTGCCTCTCTGCGCAGACTGGGGCACAGGGTTGAAG
TTTTGTTTGTCGAGGCGGAAGAAAGCGTGTTGGTCCGCCGGTTTTCCGAA
ACCAGGCGAGGACATCCTCTGAGCAATCAGGATATGACCTTGTTGGAAAG
CTTAAAGAAAGAACGGGAATGGCTGTTCCCGCTTAAAGAAATCGCCTATT
GTATCGACACTTCCAAGATGAATGCCCAACAGCTCCGCCATGCAGTCCGG
CAGTGGCTGAAGGTCGAACGTACCGGGCTGCTGGTGATTTTGGAGTCCTT
CGGGTTCAAATACGGTGTGCCGAACAACGCGGATTTTATGTTCGATATGC
GCAGCCTGCCCAACCCGTATTACGATCCCGAGTTGAGGCCTTACACCGGT
ATGGACAAGCCCGTTTGGGATTATTTGGACGGACAGCCGCTTGTGCAGGA
AATGGTTGACGACATCGAAAGGTTTCTTACGCATTGGTTACCGCGTTTGG
AGGATGAAAGCAGGAGCTACGTTACCGTCGCCATCGGTTGCACGGGAGGA
CAGCACCGTTCGGTCTATATTGTCGAAAAACTCGCCCGAAGGTTGAAAGG
GCGTTATGAATTGCTGATACGGCACAGACAGGCGCAAAACCTGTCAGACC
GCTAA
NMB0738 Protein sequence
MKIVLISGLSGSGKSVALRQMEDSGYFCVDNLPLEMLPALVSYRIERADE
TELAVSVDVRSGIDIGQAREQIASLRRLGHRVEVLFVEAEESVLVRRFSE
TRRGHPLSNQDMTLLESLKKEREWLFPLKEIAYCIDTSKMNAQQLRHAVR
QWLKVERTCLLVILESFGFKYGVPNNADFMFDMRSLPNPYYDPELREYTG
MDKPVWDYLDGQPLVQEMVDDTERFVTHWLPRLEDESRSYVTVAIGCTGG
QHRSVYTVEKLARRLKGRYELLIRHRQAQNLSDR
NMB0792 NadC family (transporter) DNA sequence
ATGAACCTGCATGCAAAGGACAAAACCCAGCATCCCGAAAACGTCGAGCT
GCTCAGTGCGCAGAAGCCGATTACCGACTTTAAGGGCCTGCTGACCACCA
TTATTTCCGCCGTCGTCTGTTTCGGCATTTACCACATCCTGCCTTACAGC
CCCGATGCCAATAAAGGTATCGCGCTGCTGATTTTCGTTGCCGCACTTTG
GTTTACCGAGGCCGTCCACATTACCGTAACCGCACTGATGGTGCCGATTC
TCGCCGTCGTACTCGGTTTCCCCGACATGGACATCAAAAAGGCGATGGCT
GATTTTTCCAACCCGATTATCTACATTTTTTTCGGCGGCTTCGCGCTTGC
CACCGCCCTGCATATGCAGCGGCTGGACCGTAAAATCGCCGTCAGCCTGT
TGCGCCTGTCGCGCGGCAATATGAAAGTGGCGGTTTTGATGTTGTTCCTC
GTTACCGCCTTTCTGTCCATGTGGATCAGCAACACCGCCACCGCCGCGAT
GATGCTGCCTCTAGCAATGGGTATGCTGAGCCACCTCGACCAGGAAAAAG
AACACAAAACCTACGTCTTCCTCCTGCTCGGCATCGCCTATTGCGCCAGC
ATCGGCGGCTTGGGCACGCTCGTCGGCTCGCCGCCCAACCTGATTGCCGC
CAAAGCCCTAAATCTGGACTTCGTCGGCTGGATGAAGCTCGGCCTGCCGA
TGATGCTGTTGATTCTGCCCTTGATGCTGCTCTCCCTGTACGTCATCCTC
AAACCTAATTTGAACGAACGCGTGGAAATCAAAGCCGAATCCATCCCTTC
GACGCTGCACCGCGTGATCGCGCTGTTGATTTTCCTTGCCACAGCCGCCG
CGTCGATATTCAGCTCCAAAATCAAAACCGCCTTCGGCATTTCCAATCCC
GACACCGTTATCGCCCTGACTGCCGCCGTCGCCGTCGTCGTCTTCGGCGT
GGCGCAATGGAAGGAAGTCGCCCGCAATACCGACTGGGGCGTCTTGATGC
TCTTCGGCGGCGGCATCAGCCTGAGCACGCTGTTGAAAACATCCGGCGCG
TCCGAAGCCTTGGGACAGCAGGTTGCCGCCACCTTTTCCGGCGCGCCCGC
ATTTTTGGTGATACTCATCGTCGCCGCCTTCATTATTTTTCTGACCGAGT
TCACCAGCAACACCGCCTCCGCCGCATTGCTTCTACCGATTTTCTCCGGC
ATCGCTATGCAGATGGGGCTGCCCGAACAAGTCTTGGTATTCGTCATCGG
CATCGGCGCATCTTGTGCCTTCATGCTGCCGGTTGCCACACGGCCTAACG
CGATTGTGTTCGGCACGGGCTTAATCAAGCAACGCGAAATGATGAATGTC
GGCATACTGCTGAACATCCTCTGCGTAGTATTGGTTCCTCTGTGGGCTTA
TGCTGTACTGATGTAA
NMB0792 Protein sequence
MNLHAKDKTQHPENVELLSAQKPITDFKGLLTTIISAVVCFGIYHTLPYS
PDANKGIALLIFVAALWFTEAVHTTVTALMVPILAVVLGFPDMDIKKAMA
DFSNPTTYIFFGGFALATALHMQRLDRKIAVSLLRLSRGNMKVAVLMLFL
VTAFLSMWISNTATAAMMLPLAMGMLSHLDQEKEHKTYVFLLLGIAYCAS
IGGLGTLVGSPPNLIAAKALNLDFVGWMKLGLPMMLLILPLMLLSLYVIL
KPNLNERVEIKAESIPWTLHRVIALLIFLATAAAWIFSSKIKTAFGISNP
DTVIALSAAVAVVVFGVAQWKEVARNTDWGVLMLFGGGISLSTLLKTSGA
SEALGQQVAATFSGAPAFLVILIVAAFIIFLTEFTSNTASAALLVPIFSG
IAMQMGLPEQVLVFVIGIGASCAFMLPVATPPNAIVFGTGLIKQREMMNV
GILLNLLCVVLVALWAYAVLM
NMB0279 DNA sequence
ATGCAACGACAAATCAAACTGAAAAATTGGCTTCAGACCGTTTATCCCGA
ACGGGACTTCGATCTGACTTTTGCGGCGGCGGATGCTGATTTCCGCCGCT
ATTTCCGTGCAACGTTTTCAGACGGCAGCAGTGTCGTCTGCATGGATGCA
CCGCCCGACAAGATGAGTGTCGCACCTTATTTGAAAGTGCAGAAACTGTT
TGACATGGTCAATGTGCCGCAGGTATTGCACGCGGACACGGATCTGGGGT
TTGTGGTATTGAACGACTTCGGCAATACGACGTTTTTGACCGCAATGCTT
CAGGAACAGGGCGAAACGGCGCAGAAAGCCCTGCTTTTGGAGGCAATCGG
CGAGTTGGTCGAATTGCAGAAGGCGAGCCGTGAAGGGGTTTTGCCCGAAT
ATGACCGTGAAACGATGTTGCGCGAAATCAACCTGTTCCCGGAATGGTTT
GTCGCAAAAGAATTGCGGCGCGAATTAACATTCAAACAACGCCAACTTTG
GCAGCAAACCGTCGATACGCTGCTGCCGCCCCTGTTGGCGCAGCCCAAAG
TCTATGTGCACCGCGACTTTATCGTCCGCAACCTGATGCTGACGCGCGGC
AGGCCGGGCGTTTTAGACTTCCAAGACGCGCTTTACGGCCCGATTTCCTA
CGATTTGGTGTCGCTGTTGCGCGATGCCTTTATCGAATGGGAAGAAGAAT
TTGTCTTGGACTTGGTTATCCGCTACTGGGAAAAGGCGCGCGCTGCCGGC
TTGCCCGTCCCCGAAGCGTTTGACGAGTTTTACCGCTGGTTCGAATGGAT
CGGCGTGCAGCGGCACTTGAAGGTTGCAGGCATCTTCGCACGCCTGTACT
ACCGCGACGGCAAAGACAAATACCGTCCGGAAATCCCGCGTTTCTTAAAC
TATCTGCGCCGCGTATCGCGCCGTTATGCCGAACTCGCCCCGCTCTACGC
GCTCTTGGTCGAACTGGTCGGCGATGAAGAACTGGAAACCGGCTTTACGT
TTTAA
NMB0279 Protein sequence
MQRQIKLKNWLQTVYPERDFDLTFAAADADFRRYFPATFSDGSSVVCMDA
PPDKMSVAPYLKVQKLFDMVNVPQVLHADTDLGFVVLNDLGNTTFLTAML
QEQGETAHKALLLEAIGELVELQKASREGVLPEYDRETMLRETNLFPEWF
VAKELGRELTFKQRQLWQQTVDTLLPPLLAQPKVYVHRDFTVRNLMLTRG
RPGVLDFQDALYGPISYDLVSLLRDAFIEWEEEFVLDLVIRYWEKARAAG
LPVPEAFDEFYRWFEWMGVQRHLKVAGIFARLYYRDGKDKYRPEIPRFLN
YLRRVSRRYAELAPLYALLVELVGDEELETGFTF
NMB2050 DNA sequence
ATGGAACTGATGACTGTTTTGCTGCCTTTGGCCGCGTTGGTGTCGGGCGT
GTTGTTTACATGGTTGCTGATGAAGCGCCGGTTTCAGGGCGAGTTTGCCG
GTTTGAACCCGCACCTGCCGGAAAAGGCGGCAAGATGTGATTTTGTCGAA
CAGGCACACGGCAAAACCGTGTCGGAATTGGCGGTGTTGGACGGGAAATA
CCGCCATTTGCAGGACGAAAATTATGCTTTGGGCAACCGTTTTTCCGCAG
CCGAAAAGCAGATTGCCCATTTGCAGGAAAAAGAGGCGGAGTCGGCGCGG
CTGAAGCAGTCGTATATCGAGTTGCAGGAAAAGGCACAGGGTTTGGCGGT
TGAAAACGAACGTTTGGCAACGCAGCTCGGACAGGAACGGAAGGCGTTTG
CCGACCAATATGCCTTGGAACGCCAAATCCGCCAAAGAATCGAAACCGAT
TTGGAAGAAAGCCGCCAAACTGTCCGCGACGTGCAAAACGACCTTTCCGA
TGTCGGCAACCGTTTTGCCGCAGCCGAAAAACAGATTGCCCATTTGCAGG
AAAAAGAGGCGGAAGCGGAGCGGTTGAGGCAGTCGCATACCGAGTTGCAG
GAAAAGGCACAGGGTTTGGCGGTTGAAAACGAACGTTTGGCAACGCAAAT
CGAACAGGAACGCCTTGCTTCTGAAGAGAAGCTGTCCTTGCTGGGCGAGG
CGCGCAAAAGTTTGAGCGATCAGTTTCAAAATCTTGCCAACACGATTTTG
GAAGAAAAAAGCCGCCGTTTTACCGAGCAGAACCGCGAGCAGCTCCATCA
GGTTTTGAACCCGCTAAACGAACGCATCCACGGTTTCGGCGAGTTGGTCA
AGCAAACCTATGATAAAGAATCGCGCGAGCCGCTGACGTTGGAAAACGAA
TTGAAACGGCTTCAGGGGTTGAACGCGCAGCTGCACAGCGAGGCAAAGGC
CCTGACCAACGCGCTGACCGGTACGCAGAATAAGGTTCAGGGCAATTGGG
GCGAGATGATTCTGGAAACGGTTTTGGAAAATTCCGGCCTTCAGAAAGGG
CGGGAATATGTGGTTCAGGCGCCATCCGTCCGAAAAGAGGAAGACGGCGG
CACGCGCCGCCTCCAGCCCGACGTTTTGGTCAACCTGCCCGACAACAAGC
AGATTGTGATTGATTCCAAGGTCTCGCTGACAGCTTATGTGCGCTACACG
CAGGCGGCGGATGCGGATACGGCGGCACGCGAACTGGCGGCACACGTTGC
CAGCATCCGTGCACACATGAAAGGCTTGTCGCTGAAGGATTACACCGATT
TGGAAGGTGTGAACACATTGGATTTCGTCTTTATGTTTATCCCTGTCGAA
CCGGCCTACCTGTTGGCGTTGCAGAATGACGCGGGCTTGTTCCAAGAGTG
TTTCGACAAACGGATTATGCTGGTCGGCCCCAGTACGCTGCTGGCGACTT
TGAGGACGGTGGCGAATATTTGGCGCAACGAACAGCAAAATCAGAACGCA
CTGGCGATTGCGGACGAAGGCGGCAAGCTGTACGACAAGTTTGTCGGCTT
CGTACAGACGCTCGAAAGCGTCGGCAAAGGCATCGATCAGGCGCAAAGCA
GTTTTCAGACGGCATTCAAGCAACTTGCCGAAGGGCGCGGGAATCTGGTC
GGACGCGCCGAGAAACTGCGTCTGTTGGGCGTGAAGGCAGGCAAACAACT
TCAACGGGATTTGGTCGAGCGTTCCAATGAAACAACGGCGTTGTCGGAAT
CTTTGGAATACGCGGCAGAAGATGAAGCAGTCTGA
NMB2050 Protein sequence
MELMTVLLPLAALVSGVLFTWLLMKGRFQGEFAGLNAHLAEKAARCDFVE
QAHGKTVSELAVLDGKYRHLQDENYALGNRFSAAEKQIAHLQEKEAESAR
LKQSYIELQEKAQGLAVENERLATQLGQERKAFADQYALERQIRQRIETD
LEESRQTVRDVQNDLSDVGNRFAAAEKQIAHLQEKEAEAERLRQSHTELQ
EKAQGLAVENERLATQIEQERLASEEKLSLLGEARKSLSDQFQNLANTIL
EEKSRRFTEQNREQLHQVLNPLNERIHGFGELVKQTYDKESRERLTLENE
LKRLQGLNAQLHSEAKALTNALTGTQNKVQGNWGEMILETVLENSGLQKG
REYVVQAASVRKEEDGGTRRLQPDVLVNLPDNKQIVIDSKVSLTAYVRYT
QAADADTAARELAAHVASIRAHMKGLSLKDYTDLEGVNTLDFVFMFIPVE
PAYLLALQNDAGLFQECFDKRTMLVGPSTLLATLRTVANTWRNEQQNQNA
LAIADEGGKLYDKFVGFVQTLESVGKGTDQAQSSFQTAFKQLAEGRGNLV
GRAEKLRLLGVKAGKQLQRDLVERSNETTALSESLEYAAEDEAV
NMB1335 CreA protein DNA sequence
ATGAACAGACTGCTACTGCTGTCTGCCGCCGTCCTGCTGACTGCCTGCGG
CAGCGGCGAAACCGATAAAATCGGACGGGCAAGTACCGTTTTCAACATAC
TGGGCAAAAACGACCGTATCGAAGTGGAAGGATTCGACGATCCCQACGTT
CAAGGGGTTGCCTGTTATATTTCGTATGCAAAAAAAGGCGGCTTGAAGGA
AATGGTCAATTTGGAAGAGGACGCGTCCGACGCATCGGTTTCGTGCGTTC
AGACGGCATCTTCGATTTCTTTTGACGAAACCGCCGTGCGCAAACCGAAA
GAAGTTTTCAAACACGGTGCGAGCTTCGCGTTCAAGAGCCGGCAGATTGT
CCGTTATTACGACCCCAAACGCAAAACCTTCGCCTATTTGGTGTACAGCG
ATAAAATCATCCAAGGCTCGCCGAAAAATTCCTTAAGCGCGGTTTCCTGT
TTCGGCGGCGGCATACCGCAAACCGATGGGGTGCAAGCCGATACTTCCGG
CAACCTGCTTGCCGGCGCCTGCATGATTTCCAACCCGATAGAAAATCTCG
ACAAACGCTGA
NMB1335 Protein sequence
MNRLLLLSAAVLLTACGSGETDKTGPASTVFNILGKNDRIEVEGFDDPDV
QGVACYISYAKKGGLKEMVNLEEDASDASVSCVQTASSISFDETAVRKPK
EVFKHGASFAFKSRQIVRYYDPKRKTFAYLVYSDKIIQGSPKNSLSAVSC
FGGGIPQTDGVQADTSGNLLAGACMISNPIENLDKR
NMB2035 DNA sequence
ATGACCGCCTTTGTCCACACCCTTTCAGACGGCATGGAACTGACCGTCGA
AATCAAGCGCCGTGCCAAGAAAAACCTGATTATCCGCCCCGCCGGCACAC
ATACCGTCCGCATCAGCGTCCCACCCTGCTTCTCCGTCTCCGCTCTAAAC
CGCTGGCTGTATGAAAACGAAGCCGTCCTGCGGCAAACACTGGCGAAAAC
ACCGCCGCCGCAAACTGCCGAAAACCGGCTGCCCGAATCCATCCTCTTCC
ACGGCAGACAGCTTGCCCTCACCGCCCATCAAGACACGCAAATCCTCCTG
ATGCCGTCTGAAATCCGTGTTCCCGAAGGCGCACCCGAAAAACAGCTTGC
GCTGCTGCGGGACTTTTTGGAACGGCAGGCGCACAGTTACCTGATTCCCC
GCCTCGAACGCCACGCCCGCACCACACAACTGTTCCCCGCCTCCTCCTCG
CTGACCTCTGCCAAAACCTTCTGGGGCGTGTGCCGCAAAACCACAGGCAT
ACGCTTCAACTGGCGGCTGGTCGGCGCACCGGAATACGTTGCCGACTATG
TCTGCATACACGAACTCTGCCACCTCGCCCATCCCGACCACAGCCCCGCC
TTTTGGGAACTGACCCGCCGCTTCGCCCCCTACACGCCCAAAGCGAAACA
GTGGCTCAAAATCCACGGCAGGGAACTTTTCGCCTTAGGCTGA
NMB2035 Protein sequence
MTAFVHTLSDGMELTVEIKRRAKKNLIIRPAGTHTVRISVPPCFSVSALN
RWLYENEAVLRQTLAKTPPPQTAENRLPESILFHGRQLALTAHQDTQILL
MPSEIRVPEGAPEKQLALLRDFLERQAHSYLTPRLERHARTTQLFPASSS
LTSAKTFWGVCRKTTGTRFNWRLVGAPEYVADYVCIHELCHLAHPDHSPA
FWELTRRFAPYTPKAKQWLKIHGRELFALG
NMB1351 Fmu and Fmv protein DNA sequence
ATGAACGCCGCACAACTCGACCATACCGCCAAAGTTTTGGCTGAAATGCT
GACTTTCAAACAGCCTGCCGATGCCGTCCTCTCCGCCTATTTCCGCGAAC
ACAAAAAGCTCGGCAGTCAAGATCGCCACGAAATCGCCGAAACCGCCTTT
GCCGCGCTGCGCCACTATCAAAAAATCAGTACCGCCCTACGCCGTCCGCA
CGCGCAGCCGCGCAAAGCCGCTCTCGCCGCACTGGTTCTCGGCAGAAGCA
CCAACATCAGCCAAATCAAAGACCTGCTTGATGAAGAAGAAACAGCGTTC
CTCGGCAATTTGAAAGCCCGTAAAACCGAGTTTTCAGACAGCCTGAATAC
CGCCGCAGAATTGCCGCAATGGCTGGTGGAACAACTGAAACAGCATTGGC
GCGAAGAAGAAATCCTCGCTTTCGGCCGCAGCATCAACCAGCCTGCCCCG
CTCGACATCCGCGTCAACACTTTGAAAGGCAAACGCGATAAAGTGCTGCC
GCTGTTGCAAGCCGAAAGTGCCGATGCAGAGGCAACGCCTTATTCGCCTT
GGGGCATCCGCCTGAAAAACAAAATCGCGCTTAACAAACACGAACTGTTT
TTAGACGGCACACTGGAAGTCCAAGACGAAGGCAGCCAGCTGCTTGCCTT
ATTGGTGGGCGCAAAACGAGGCGAAATCATTGTCGATTTCTGTGCCGGTG
CCGGCGGTAAAACCTTGGCTGTCGGTGCGCAAATGGCGAACAAAGGCAGA
ATCTACGCCTTCGATATCGCCGAAAAACGCCTTGCCAACCTCAAACCGCG
TATGACCCGCGCCGGACTGACCAATATCCACCCCGAACGCATCGGCAGCG
AACACGATGCCCGTATCGCCCGACTGGCAGGCAAAGCCGACCGTGTGTTG
GTGGACGCGCCCTGCTCCGGTTTGGGCACTTTACGCCGCAATCCCGACCT
CAAATACCGCCAATCCGCCGAAACCGTCGCCAACCTTTTGGAACAGCAAC
ACAGCATCCTCGATGCCGCCTCCAAACTGGTAAAACCGCAAGGACGTTTG
GTGTACGCCACTTGCAGCATCCTGCCCGAAGAAAACGAGCTGCAAGTCGA
ACGTTTCCTGTCCGAACATCCCGAATTTGAACCCGTCAACTGCGCCGAAC
TGCTTGCCGGTTTGAAAATCGATTTGGATACCGGCAAATACCTGCGCCTC
AACTCCGCCCGACACCAAACCGACGGCTTCTTCGCCGCCGTATTGCAACG
CAAATAA
NMB1351 Protein sequence
MNAAQLDHTAKVLAEMLTFKQPADAVLSAYFREHKKLGSQDRHEIAETAF
AALRHYQKISTALRRPHAQPRKAALAALVLGRSTNTSQIKDLLDEEETAF
LGNLKARKTEFSDSLNTAAELPQWLVEQLKQNWREEEILAFGRSINQPAP
LDIRVNTLKGKRDKVLPLLQAESADAEATPYSPWGTRLKNKTALNKHELF
LDGTLEVQDEGSQLLALLVGAKRGEIIVDFCAGAGGKTLAVGAQMANKGR
IYAFDIAEKRLANLKPRMTRAGLTNTHPERIGSEHDARIARLAGKADRVL
VDAPCSGLGTLRRNPDLKYRQSAETVANLLEQQHSILDAASKLVKPQGRL
VYATCSILPEENELQVERFLSEHPEFEPVNCAELLAGLKIDLDTGKYLRL
NSARHQTDGFFAAVLQRK
NMB1574 IlvC DNA sequence
ATGCAAGTCTATTACGATAAAGATGCCGATCTGTCCCTAATCAAAGGCAA
AACCGTTGCCATCATCGGTTACGGTTCGCAAGGTCATGCCCATGCCGCCA
ACCTGAAAGATTCGGGTGTAAACGTGGTGATTGGTCTGCGCCAAGGTTCT
TCTTGGAAAAAAGCCGAAGCAGCCGGTCATGTCGTCAAAACCGTTGCTGA
AGCGACCAAAGAAGCCGATGTCGTTATGCTGCTGCTGCCTGACGAAACCA
TGCCTGCCGTCTATCACGCCGAAGTTACAGCCAATTTGAAAGAAGGCGCA
ACGCTGGCATTTGCACACGGCTTCAACGTGCACTACAACCAAATCGTTCC
GCGTGCCGACTTGGACGTGATTATGGTTGCCCCCAAAGGTCCGGGCCATA
CCGTACGCAGTGAATACAAACGCGGCGGCGGCGTGCCTTCTCTGATTGCC
GTTTACCAAGACAATTCCGGCAAAGCCAAAGACATCGCCCTGTCTTATGC
GGCTGCCAACGGCGGCACCAAAGGCGGTGTGATTGAAACCACTTTCCGCG
AAGAAACCGAAACCGATCTGTTCGGCGAACAAGCCGTATTGTGCGGCGGC
GTGGTCGAGTTGATCAAGGCGGGTTTTGAAACCCTGACCGAAGCCGGTTA
CGCGCCTGAAATGGCTTACTTCGAATGTCTGCACGAAATGAAACTGATCG
TTGACCTGATTTTCGAAGGCGGTATTGCCAATATGAACTACTCCATTTCC
AACAATGCGGAGTACGGCGAATACGTTACCGGCCCTGAAGTGGTCAATGC
TTCCAGCAAAGAAGCCATGCGCAATGCCCTGAAACGCATTCAAACCGGCG
AATACGCAAAAATGTTTATCCAAGAGGGTAATGTCAACTATGCGTCTATG
ACTGCCCGCCGCCGTCTGAATGCCGACCACCAAGTTGAAAAAGTCGGCGC
ACAACTGCGTGCCATGATGCCTTGGATTACTGCCAACAAATTGGTTGACC
AAGACAAAAACTGA
NMB1574 Protein sequence
MQVYYDKDADLSLIKGKTVAIIGYGSQGHAHAANLKDSGVNVVIGLRQCS
SWKKAEAAGHVVKTVAEATKEADVVNLLLPDETMPAVYHAEVTANLKEGA
TLAFAHGFNVHYNQIVPRADLDVIMVAPKGPGHTVRSEYKRGGGVPSLIA
VYQDNSGKAKDIALSYAAANGGTKGGVIETTFREETETDLFGEQAVLCGG
VVELIKAGFETLTEAGYAPEMAYFECLHEMKLIVDLIFEGGIANMNYSIS
NNAEYGEYVTGPEVVNASSKEAMRNALKRIQTGEYAKMFIQEGNVNYASM
TARRRLNADHQVEKVGAQLRAMMPWITANKLVDQDKN
NMB1298 rsuA DNA sequence
ATGAAACTTATCAAATACCTGCAATATCAAGGCATAGGAAGCCGCAAGCA
GTGCCAATGGCTGATTGCCGGCGGTTATGTTTTCATGAACGGAACCTGCA
TGGACGACACCGATGCAGACATCGATTCCTCATCCGTCGAAACGTTGGAT
ATTGACGGGGAAGCAGTAACCGTCGTTCCCGAACCCTATTTCTACATCAT
GCTCAACAAGCCTGAAGATTACGAAACTTCGCACAAACCCAAGCACTACC
GCAGCGTATTCAGCCTGTTCCCCGACAATATGCGGAACATCGATATGCAG
GCGGTCGGCAGGCTGGATGCAGATACGACCGGCGTATTGCTGATTACCAA
CGACGGCAAACTGAACCACAGCCTGACTTCGCCGAGCAGAAAAATTCCCA
AGCTGTACGAAGTAACGCTCAAACACCCCACAGGAGAAACGCTCTGCGAA
ACCTTGAAAAACGGCGTGCTGCTCCACGACGAAAACGAAACCGTTTGTGC
CGCCGATGCCGTTTTGAAAAACCCGACCACCCTGCTGCTGACCATTACCG
AAGGAAAATACCACCAAGTCAAACGCATGATCGCCGCCGCCGGCAACCGC
GTGCAACACCTTCATCGCCGGCGATTCGCACATCTGGAAACAGAAAACCT
CAAACCCGGGGAATGGAAATTTATCGAATGTCCAAAATTCTGA
NMB1298 Protein sequence
MKLIKYLQYQGIGSRKQCQWLIAGGYVFTNGTCMDDTDADIDSSSVETLD
TDGEAVTVVPEPYFYIMLNKPEDYETSHKPKHYRSVFSLFPDNMRNIDMQ
AVGRLDADTTGVLLITNDGKLNHSLTSPSRKIPKLYEVTLKHPTGETLCE
TLKNGVLLHDENETVCAADAVLKNPTTLLLTITEGKYHQVKRMIAAAGNR
VQHLHRRRFAHLETENLKPGEWKFIECPKF
NMB1856 Lys R family (transcription regulator)
DNA sequence
ATGAAAACCAATTCAGAAGAACTGACCGTATTTGTTCAAGTGGTGGAAAG
CGGCAGCTTCAGCCGTGCGGCGGAGCAGTTGGCGATGGCAAATTCTGCCG
TAAGCCGCATCGTCAAACGGCTGGAGGAAAAGTTGGGTGTGAACCTGCTC
AACCGCACCACGCGGCAACTCAGTCTGACGGAAGAAGGCGCGCAATATTT
CCGCCGCGCGCAGAGAATCCTGCAAGAAATGGCAGCGGCGGAAACCGAAA
TGCTGGCAGTGCACGAAATACCGCAAGGCGTGTTGAGCGTGGATTCCGCG
ATGCCGATGGTGCTGCATCTGCTGGCGCCGCTGGCAGCAAAATTCAACGA
ACGCTATCCGCATATCCGACTTTCGCTCGTTTCTTCCGAAGGCTATATCA
ATCTGATTGAACGCAAAGTCGATATTGGCTTAGGGGCGGGAGAATTGGAG
GATTGCGGGGTGCGTGGACGGCATGTGTTTGACAGGGGCTTCGGGGTAAT
GGCCAGTGCTGAATACGTGGCAAAACAGGGCAGGGGGCAATGTACAGAAG
AGCTTGCGGGGGACGAATGTTTAGGGTTGAGGGAAGCCGGTTGTGTAAAT
ACATGGGCGGTTTTAGATGCGCAGGGAAATCCCTATAAGATTTCACCGCA
CTTTACCGCCAGCAGGGGTGAAATGTTAGGCTGGTTGTGCGTTTCAGGTT
GGGGTATTGTTTGCTTATGAGATTTTTTGGTTGAGAACGACATCGCTGAA
GGAAAGTTAATTCCCGTGGTCGGCGAAGAAACCTCCGATAAAACACACCC
CTTTAATGCTGTTTATTACAGCGATAAAGCCGTGAATCTCGGCTTACGCG
TATTTTTGGATTTTTTAGTGGAGGAAGTGGGAAACAATCTGTGTGGATAA
NMB1856 Protein sequence
MKTNSEELTVFVQVVESGSFSPAAEQLAMANSAVSRIVKRLEEKLGVNLL
NRTTRQLSLTEEGAQYFRRAQRILQEMAAAETEMLAVHEIPQGVLSVDSA
MPMVLHLLAPLAAKFNERYPHIRLSLVSSEGYINLIERKVDIALPAGELD
DSGLRARHLFDSRFRVIASPEYLAKHGTPQSTEELAGHQCLGFTEPGSLN
TWAVLDAQGNPYKTSPHFTASSGETLRSLCLSGCGIVCLSDFLVDNDIAE
GKLIPLLAEQTSDKTHPFNAVYYSDKAVNLRLRVFLDFLVEELGNNLCG
NMB0119 DNA sequence
ATGATGAAGGATTTGAATTTGAGCAACAGCCTGTTCAAAGGCTACAACGA
CAAACATGGCTTAATGATTTGTGGCTATGAATGGGGTTGGAGTAAAGCCG
ATGAGGCTGCTTATGTAGCAGGTGAATACAAACTCCCTGAAAACAAAATC
GACCATACATTTGCAAACAAATCCCTCTATTTCGGAGAGCAGGCAAAAAA
GTGGCGTTACGACAATACGATAAAAAATTGGTTTGAAATGTGGGGACACC
CCTTAGACGAAAATGGATTGGGCGGTGCATTTGAAAAATCCCTGGTTCAA
ACCAACTGGGCTGCTACACAGGGCAACACTATCGACAATCCCGACAAGTT
CACACAACCCGAGCACATCGATAATTTTCTCTACCACATCGAAAAACTGC
GTCCGAAAGTCATCCTCTTCATGGGCAGCAGGTTGGCGGATTTTCTGAAC
AACCAAAATGTACTGCCACGCTTCGAGCAGTTGGTCGGTAAGCAGACCAA
ACCGCTGGAGACGGTGCAAAAAGAATTTGACGGTACACGTTTCAATGTCA
AATTCCAATCGTTTGAAGATTGCGAAGTCGTCTGCTTTCCCCATCCCAGT
GCCAGTCGCGGTCTATCTTACGATTACATCGCCTTGTTTGCGCCTGAAAT
GAACCGGATTTTATCGGACTTTAAAACAACACGCGGATTCAAATAA
NMB0119 Protein sequence
MMKDLNLSNSLFKGYNDKHGLMICGYEWGWSKADEAAYVAGEYKLPENKI
DHTFANKSLYFGEQAKKWRYDNTIKNWFEMWGHPLDENGLGGAFEKSLVQ
TNWAATQGNTIDNPDKFTQPEHIDNFLYHIEKLRPKVILFMGSRLADFLN
NQNVLPRFEQLVGKQTKPLETVQKEFDGTRFNVKFQSFEDCEVVCFPHPS
ASRGLSYDYIALFAPEMNRILSDFKTTRGFK
NMB1705 rfaK DNA sequence
ATGGAAAAAGAATTCAGGATATTAAATATCGTATCGGCCAAGATTTGGGG
TGGAGGCGAACAATATGTCTATGATGTTTCAAAAGCATTGGGGCTTCGGG
GCTGCACAATGTTTACCGCCGTCAATAAAAATAATGAATTGATGCACAGG
CGATTTTCCGAAGTTTCTTCCGTTTTCACAACGCGCCTTCACACGCTCAA
CGGGCTGTTTTCGCTCTACGCACTTACCCGCTTTATCCGGAAAAACCGCA
TTTCCCACCTGATGATACACACCGGCAAAATTGCCGCCTTATCCATACTT
TTGAAAAAACTGACCGGGGTGCGCCTGATATTTGTCAAACATAATGTCGT
CGCCAACAAAACCGATTTTTACCACCGCCTGATACAGAAAAACACAGACC
GCTTTATTTGCGTTTCCCGTCTGGTTTACGATGTGCAAACCGCCGACAAT
CCCTTTAAAGAAAAATACCGGATTGTTCATAACGGTATCGATACCGGCCG
TTTCCCTCCCTCTCAAGAAAAACCCGACAGCCGTTTTTTTACCGTCGCCT
ACGCCGGCAGGATCAGTCCAGAAAAAGGATTGGAAAACCTGATTGAAGCC
TGTGTGATACTCCATCGGAAATATCCTCAAATCAGGCTCAAATTGGCAGG
GGACGGACATCCGGATTATATGTGCCGCCTGAAGCGGGACGTATCTGCTT
CAGGAGCAGAACCATTTGTTTCTTTTGAAGGGTTTACCGAAAAACTTGCT
TCGTTTTACCGCCAAAGCGATGTCGTGGTTTTGCCCAGCCTCGTCCCGGA
GGCATTCGGTTTGTCATTATGCGAGGCGATGTACTGCCGAACGGCGGTGA
TTTCCAATACTTTGGGGGCGCAAAAGGAAATTGTCGAACATCATCAATCG
GGGATTCTGCTGGACAGGCTGACACCTGAATCTTTGGCGGACGAAATCGA
ACGCCTCGTCTTGAACCCTGAAACGAAAAACGCACTGGCAACGGCAGCTC
ATCAATGCGTCGCCGCCCGTTTTACCATCAACCATACCGCCGACAAATTA
TTGGATGCAATATAA
NMB1705 Protein sequence
MEKEFRILNIVSAKIWGGGEQYVYDVSKALGLRGCTMFTAVNKNNELMHR
RFSEVSSVFTTRLHTLNGLFSLYALTRFIRKNRISHLMIHTGKIAALSIL
LKKLTGVRLIFVKHNVVANKTDFYHRLIQKNTDRFICVSRLVYDVQTADN
PFKEKYRIVHNGIDTGRFPPSQEKPDSRFFTVAYAGRISPEKGLENLIEA
CVTLHRKYPQTRLKLAGDGHPDYMCRLKRDVSASGAEPFVSFEGFTEKLA
SFYRQSDVVVLPSLVPEAFGLSLCEAMYCRTAVISNTLGAQKEIVEHHQS
GILLDRLTPESLADEIERLVLNPETKNALATAAHQCVAARFTINHTADKL
LDAI
NMB2065 Hemk protein DNA sequence
ATGCAGGAACAGAATCGGAAACCAAGTTTTCCCATAGTGATGTTGCTGGT
GTCGGTTGCCCTGTGGATAGCGTCTTTATCCAATGTTGCATTTTATTTGG
GCAATCATGGAAGCATGGAGGGTTTGACCGTTTTGATTTTGGGGTCGATA
TTTGCTTCTTTGGATATCAGGTATTGTGCGGTCTATGCGAATTATGTTTG
GTTGGCGGCCATTGTTTTGCTGGCGTTGCGGAAGAAGGTCGTGCCTGTCC
ATGCGGCACTTTGGGGCTTGGCGTTGGTGGCTTTCAGTGTGAAAGCCGTA
TACGTCGATGAAGCAGGGAATACATCGGATATTCTGCGCTACGGTGCAGG
ATTTTATTTGTGGTATGCCGCATTTGCGGTTGCCACCATCGGTACGTTTG
CCGGAAAGAATAAGGAAAGAAAAGCCGCATCAGCGGCAGACGGGATAAAA
ATGACGTTTGATAAATGGTTGGGCTTGTCAAAACTGCCTAAAAATGACGC
AAGAATGCTGCTACAATATGTTTCGGAATATACGCGCGTGCAGTTGTTGA
CGCGGGGCGGGGAAGAAATGCCGGACGAAGTCCGACAGCGGGCGGACAGG
CTGGCGCAACGCCGTCTGAACGGCGAGCCGGTTGCCTATATTTTAGGTGT
GCGCGAATTTTATGGCAGACGCTTTACAGTCAATCCGAGCGTGCTGATTC
CGCGCCCCGAAACCGAACATTTGGTCGAAGCCGTATTGGCGCGCCTGCCC
GAAAACGGGCGCGTGTGGGATTTGGGGACGGGCAGCGGCGCGGTTGCCGT
AACCGTCGCGCTCGAACGCCCCGATGCGTTTGTGCGCGCATCCGACATCA
GCCCGCCCGCCCTTGAAACGGCGCGGAAAAATGCGGCGGATTTGGGCGCG
CGGGTCGAATTTGCACACGGTTCGTGGTTCGACACCGATATGCCGTCTGA
AGGGAAATGGGACATCATCGTGTCCAACCCGCCCTATATCGAAAACGGCG
ATAAACATTTGTTGCAAGGCGATTTGCGGTTTGAGCCGCAAATCGCGCTG
ACCGACTTTTCAGACGGCCTAAGCTGCATCCGCACCTTGGCGCAAGGCGC
GCCCGACCGTTTGGCGGAAGGCGGTTTTTTATTGCTGGAACACGGTTTCG
ATCAGGGCGCGGCGGTGCGCGGCGTGTTGGCGGAGAATGGTTTTTCAGGA
GTGGAAACCCTGCCGGATTTGGCGGGTTTGGACAGGGTTACGCTGGGGAA
GTATATGAAGCATTTGAAATAA
NMB2065 Protein sequence
MQEQNRKPSFPTVMLLVSVALWIASLSNVAFYLGNHGSMEGLTVLILGSI
FASLDIRYCAVYANYVWLAAIVLLALRKKVVPVHAALWGLALVAFSVKAV
YVDEAGNTSDTVRYGAGFYLWYAAFAVATIGTFAGKNKERKAASAADGIK
MTFDKWLGLSKLPKNEARMLLQYVSEYTRVQLLTRGGEEMPDEVRQRADR
LAQRRLNGEPVAYTLGVREFYGRRFTVNPSVLIPRPETEHLVEAVLARLP
ENGRVWDLGTGSGAVAVTVALERPDAFVPASDISPPALETARKNAADLGA
RVEFAHGSWFDTDMPSEGKWDIIVSNPPYIENGDKHLLQGDLRFEPQIAL
TDFSDGLSCIRTLAQGAPDRLAEGGFLLLEHGFDQGAAVRGVLAENGFSG
VETLPDLAGLDRVTLGKYMKHLK

Mutants Selected by Vacinee's 17 D Sera (Screened Once Only)

NMB0339 DNA sequence
ATGGACAACGAATTGTGGATTATCCTGCTGCCGATTATCCTTTTGCCCGT
CTTCTTCGCGATGGGCTGGTTTGCCGCCCGCGTGGATATGAAAACCGTAT
TGAAGCAGGCAAAAAGCATCCCTTCGGGATTTTATAAAAGCTTGGACGCT
TTGGTCGACCGCAACAGCGGGCGCGCGGCAAGGGAGTTGGCGGAAGTCGT
CGACGGCCGGCCGCAATCGTATGATTTGAACCTCACCCTCGGCAAACTTT
ACCGCCAGCGTGGCGAAAACGACAAAGCCATCAACATACACCGGACAATG
CTCGATTCTCCCGATACGGTCGGCGAAAAGCGCGCGCGCGTCCTGTTTGA
ATTGGCGCAAAACTACCAAAGTGCGGGGTTGGTCGATCGTGCCGAACAGA
TTTTTTTGGGGCTGCAAGACGGTAAAATGGCGCGTGAAGCCAGACAGCAC
CTGCTCAATATCTACCAACAGGACAGGGATTGGGAAAAAGCGGTTGAAAC
CGCCCGGCTGCTCAGCCATGACGATCAGACCTATCAGTTTGAAATCGCCC
AGTTTTATTGCGAACTTGCCCAAGCCGCGCTGTTCAAGTCCAATTTCGAT
GTCGCGCGTTTCAATGTCGGCAAGGCACTCGAAGCCAACAAAAAATGCAC
CCGCGCCAACATGATTTTGGGCGACATCGAACACCGACAAGGCAATTTCC
CTGCCGCCGTCGAAGCCTATGCCGCCATCGAGCAGCAAAACCATGCATAC
TTGAGCATGGTCGGCGAGAAGCTTTACGAAGCCTATGCCGCGCAGGGAAA
ACCTGAAGAAGGCTTGAACCGTCTGACAGGATATATGCAGACGTTTCCCG
AACTTGACCTGATCAATGTCGTGTACGAGAAATCCCTGCTGCTTAAGTGC
GAGAAAGAAGCCGCGCAAACCGCCGTCGAGCTTGTCCGCCGCAAGCCCGA
CCTTAACGGCGTGTACCGCCTGCTCGGTTTGAAACTCAGCGATATGAATC
CGGCTTGGAAAGCCGATGCCGACATGATGCGTTCGGTTATCGGACGGCAG
CTACAGCGCAGCGTGATGTACCGTTGCCGCAACTGCCACTTCAAATCCCA
AGTCTTTTTCTGGCACTGCCCCGCCTGCAACAAATGGCAGACGTTTACCC
CGAATAAAATCGAAGTTTAA
NM4B0339 Protein sequence
MDNELWIILLPIILLPVFFAMGWFAARVDMKTVLKQAKSIPSGFYKSLDA
LVDRNSGRAARELAEVVDGRPQSYDLNLTLGKLYRQRGENDKAINIHRTM
LDSPDTVGEKRARVLFELAQNYQSAGLVDPAEQIFLGLQDGKMAREARQH
LLNIYQQDRDWEKAVETARLLSHDDQTYQFEIAQFYCELAQAALFKSNFD
VARFNVGKALEANKKCTRANMILGDIEHRQGNFPAAVEAYAAIEQQNHAY
LSMVGEKLYEAYAAQGKPEEGLNRLTGYMQTFPELDLINVVYEKSLLLKC
EKEAAQTAVELVRRKPDLNGVYRLLGLKLSDMNPAWKADADMMRSVIGRQ
LQRSVMYRCRNCHFKSQVFFWHCPACNKWQTFTPNKIEV

Selection with Patient's Sera

We have a collection of acute and convalescent sera available to us for screening. This is from individuals infected with different serogroup of N. meningitidis. Screens have been performed with acute (A) or convalescent (C) sera. The period between the acute infection and collection of sera was from 2 weeks to 3 months.

NMB0401 putA DNA sequence
ATGTTTCATTTTGCATTTCCGGCACAAACTGCCCTGCGCCAAGCGATAAC
CGATGCCTACCGCCGTAATGAAATCGAAGCCGTACAGGATATGTTGCAAC
GTGCACAGATGAGCGACGAAGAGCGCAACGCCGCCTCCGAGCTTGCCCGC
CGTTTGGTTACCCAAGTCCGCGCCGGCCGCACCAAAGCCGGCGGCGTGGA
TGCGCTGATGCACGAGTTTTCACTCTCCAGCGAAGAAGGCATCGCGCTGA
TGTGTCTGGCAGAAGCCCTGCTGCGTATCCCCGACAACGCCACGCGCGAC
CGCCTGATTGCCGACAAGATTTCAGACGGCAACTGGAAAAGCCATTTGAA
CAACAGCCCTTCCCTCTTCGTCAATGCTGCCGCCTGGGGCCTGCTGATTA
CCGGCAAACTGACCGCCACAAACGACAAACAAATGAGTTCCGCACTCAGC
CGCCTGATCAGCAAAGGCGGCGCACCGCTCATCCGCCAGGCGTAAAATTA
CGCCATGCGCCTTCTGGGCAAACAGTTCGTAACCGGACAGACCATTGAAG
AAGCCCTGCAAAACGGCAAAGAACGCGAAAAAATGGGCTACCGCTTCTCC
TTCGATATGTTGGGCGAAGCCGCCTACACCCAAGCCGATGCCGACCGCTA
CTACCGCGACTATGTCGAAGCCATCCACGCCATCGGCAAAGATGCGGCAG
GACAAGGCGTTTACGAAGGTAACGGTATTTCCGTCAAACTTTCCGCCATC
CATCCGCGCTACTCGCGCACCCAACACGGCCGCGTCATGGGCGAACTGTT
GCCGCGCCTGAAAGAGCTGTTCCTTTTGGGTAAAAAATACGATATCGGTA
TCAACATCGATGCCGAAGAAGCCAACCGTCTGGAGCTGTCTTTGGATTTG
ATGGAGGCTTTGGTTTCAGACCCTGACTTGGCTGGCTACAAAGGTATCGG
TTTCGTTGTCCAAGCCTACCAAAAACGTTGTCCGTTCGTTATCGACTACC
TGATCGACCTTGCCCGCCGCAACAACCAAAAACTAATGATCCGCCTCGTC
AAAGGCGCGTATTGGGACAGCGAAATCAAATGGGCGCAAGTGGACGGCTT
GAACGGCTATCCGACCTACACCCGCAAAGTCCACACCGACATCTCCTACC
TCGCCTGCGCGCGCAAACTGCTTTCCGCGCAAGACGCGGTATTCCCGCAA
TTTGCCACCCACAACGCCTACACTTTGGGCGCAATCTACCAAATGGGTAA
AGGCAAAGATTTTGAACACCAATGCCTGCACGGTATGGGCGAAACCCTGT
ACGACCAAGTCGTCGGCCCGCAAAACTTAGGCCGCCGCGTGCGCGTGTAC
GCCCCAGTCGGCACACACGAAACCCTGCTCGCCTACTTGGTGCGCCGCCT
GTTGGAAAACGGCGCGAACTCGTCTTTCGTCAACCAAATCGTCGATGAAA
ACATCAGCATCGACACGCTCATCCGCAGCCCGTTCGACACCATCGCCGAA
CAAGGCATCCACCTGCACAACGCCCTGCCGCTGCCGCGCGATTTGTACGG
CAAATGCCGTCTGAACTCGCAAGGCGTGGACTTGAGCAACGAAAACGTAT
TGCAGCAGCTTCAAGAACAGATGAACAAAGCCGCCGCGCAAGACTTCCAC
GCCGCATCCATCGTCAACGGCAAAGCCCGCGATGTCGGCGAAGCGCAACC
GATTAAAAACCCTGCCGACCACGACGACATCGTCGGCACAGTCAGCTTTG
CCGATGCCGCGCTTGCCCAAGAAGCGGTTGGCGCAGCCGTTGCCGCGTTC
CCCGAATGGAGTGCGACACCTGCCGCCGAACGCGCCGCCTGCCTGCGCCG
TTTTGCCGATTTGCTGGAGCAGCACACCCCAGCACTGATGATGCTTGCCG
TGCGCGAAGCAGGCAAAACGCTGAACAACGCCATTGCCGAAGTGCGCGAA
GCCGTCGATTTCTGCCGCTACTACGCAAACGAAGCCGAACATACCCTGCC
TCAAGACGCAAAAGCCGTCGGCGCGATTGTCGCCATCAGCCCGTGGAACT
TCCCGCTCGCCATCTTTACCGGCGAAGTCGTTTCCGCATTGGCGGCAGGC
AACACCGTCATCGCCAAACCCGCCGAACAAACCAGCCTGATTGCCGGTTA
TGCCGTTTCCCTCATGCACGAAGCCGGCATCCCGACTTCCGCCCTGCAAC
TCGTCCTCGGCGCAGGCGACGTGGGTGCGGCATTGACCAACGATGCCCGC
ATCGGCGGCGTGATTTTCACCGGCTCGACCGAAGTGGCGCGCCTGATCAA
CAAAGCCCTTGCCAAACGCGGCGACAATCCCGTCCTGATTGCCGAAACCG
GCGGACAAAACGCCATGATTGTCGATTCCACCGCACTTGCCGAGCAAGTC
TGCGCCGACGTATTGAACTCCGCCTTCGACAGCGCGGGACAACGCTGCTC
CGCCCTGCGCATTTTGTGCGTCCAAGAAGACGTTGCCGACCGTATGCTCG
ACATGATCAAAGGCGCTATGGACGAACTCGTCGTCGGCAAACCGATTCAG
CTCACTACCGATGTCGGCCCCGTCATCGATGCCGAAGCACAGCAAAACCT
GTTGAACCACATCAACAAAATGAAAGGTGTTGCCAAGTCCTACCACGAAG
TCAAAACCGCCGCCGATGTCGATTCCAAAAAATCCACGTTCGTTCGCCCC
ATCCTGTTTGAATTGAACAACCTCAACGAACTGCAACGCGAAGTCTTCGG
TCCCGTCCTGCACGTCGTCCGCTACCGCGCCGACGAACTCGACAACGTCA
TCGACCAAATCAACAGCAAAGGCTACGCCCTGACCCACGGCGTACACAGC
CGCATCGAAGGCACGGTACGCCACATCCGCAGCCGCATCGAAGCCGGCAA
CGTTTACGTCAACCGCAACATCGTCGGCGCAGTCGTCGGCGTACAGCCCT
TCGGCGGACACGGTCTGTCCGGCACAGGCCCCAAAGCAGGCGGTTCGTTC
TACCTGCAAAAACTGACCCGCGCCGGCGAATGGGTTGCCCCGACCCTGAG
CCAAATCGGACAGGCGGACGAAGCCGCACTCAAACGCCTCGAAGCACTGG
TTCACAAACTACCGTTCAACGCCGAAGAGAAAAAAGCCGCAGCGGCCGCT
TTGGGACACGCCCGCATCCGCACCCTGCGCCGTGCCGAAACCGTCCTTAC
CGGACCGACCGGCGAGCGCAACAGCATCTCATGGCACGCGCCCAAACGCG
TTTGGATACACGGCGGCAGCACGGTTCAAGCCTTTGCCGCACTGACCGAA
CTTGCCGCCTCCGGCATACAGGCAGTGGTCGAACCCGACAGCCCCTTGGC
TTCCTACACTGCCGACTTGGAAGGTCTGCTGCTGGTCAACGGCAAACCCG
AAACCGCCGGCATCAGCCACGTTGCCGCCCTGTCGCCTTTGGACAGCGCG
CGCAAACAGGAACTTGCCGCCCACGACGGCGCACTCATCCGCATCCTCCC
TTCGGAAAACGGACTCGACATCCTGCAAGTGTTTGAAGAAATCTCTTGCA
GCGTCAACACCACAGCCGCCGGCGGCAACGCCAGCCTGATGGCGGTCGCC
GACTGA
NMB0401 Protein sequence
MFHFAFPAQTALRQAITDAYRRNEIEAVQDMLQRAQMSDEERNAASELAR
RLVTQVRAGRTKAGGVDALMHEFSLSSEEGIALMCLAEALLRIPDNATRD
RLIADKISDGNWKSHLNNSPSLFVNAAAWGLLITGKLTATNDKQMSSALS
RLISKGGAPLIRQGVNYAMRLLGKQFVTGQTIEEALQNGKEREKMGYRFS
FDMLGEAAYTQADADRYYRDYVEAIHAIGKDAAGQGVYEGNGISVKLSAI
HPRYSRTQHGRVMGELLPRLKELFLLGKKYDIGINIDAEEANRLELSLDL
MEALVSDPDLAGYKGIGFVVQAYQKRCPFVIDYLIDLARRNNQKLMIRLV
KGAYWDSEIKWAQVDGLNGYPTYTRKVHTDISYLACARKLLSAQDAVFPQ
FATHNAYTLGAIYQMGKGKDFEHQCLHGMGETLYDQVVGPQNLGRRVRVY
APVGTHETLLAYLVRRLLENGAHSSFVNQIVDENISIDTLIRSPFDTIAE
QGIHLHNALPLPRDLYGKCRLNSQGVDLSNENVLQQLQEQMNKAAAQDFH
AASIVNGKARDVGEAQPIKNPADHDDIVGTVSFADAALAQEAVGAAVAAF
PEWSATPAAERAACLRRFADLLEQHTPALMMLAVREAGKTLNNATAEVRE
AVDFCRYYANEAEHTLPQDAKAVGAIVAISPWNFPLAIFTGEVVSALAAG
NTVIAKPAEQTSLIAGYAVSLMHEAGIPTSALQLVLGAGDVGAALTNDAR
IGGVIFTGSTEVARLINKALAKRGDNPVLIAETGGQNAMTVDSTALAEQV
CADVLNSAFDSAGQRCSALRILCVQEDVADRMLDMIKGAMDELVVGKPIQ
LTTDVGPVTDAEAQQNLLNHINKMKGVAKSYHEVKTAADVDSKKSTFVRP
ILFELNNLNELQREVFGPVLHVVRYRADELDNVIDQINSKGYALTHGVHS
RIEGTVRHIRSRTEAGNVYVNRNIVGAVVGVQPFGGHGLSGTGPKAGGSF
YLQKLTRAGEWVAPTLSQIGQADEAALKRLEALVHKLPFNAEEKKAAAAA
LGHARTRTLRPAETVLTGPTGERNSISWHAPKRVWTHGGSTVQAFAALTE
LAASGIQAVVEPDSPLASYTADLEGLLLVNGKPETAGISHVAALSPLDSA
RKQELAAHDGALTRILPSENCLDTLQVFEEISCSVNTTAAGGNASLMAVA
D

NMB1335 CreA

DNA and Protein sequences given above

NMB1467 PPX DNA sequence
ATGACCACCACCCCCGCAAACGTCCTCGCCTCCGTCGATTTGGGTTCCAA
CAGTTTCCGCCTCCAGATTTGCGAAAACAACAACGGACAATTAAAAGTCA
TCGATTCGTTCAAACAGATGGTGCGCTTCGCCGCCGGACTGGACGAACAG
AAAAATCTGAGTGCCGCTTCCCAAGAACAGGCTTTGGACTGTCTGGCAAA
ATTCGGCGAACGCCTGCGCGGCTTCCGCCCTGAACAGGTACGCGCCGTGG
CAACCAACACATTCCGCGTTGCCAAAAACATCGCAGATTTCCTTCCCAAA
GCCGAAGCGGCATTGGGTTTCCCCATCGAAATCATCGCCGGGCGCGAAGA
GGCGCGGCTGATTTATACCGGCGTGATCCACACCCTCCCCCCGGGCGGCG
GCAAAATGCTGGTTATCGACATCGGCGGCGGTTCGACAGAATTTGTCATC
GGCTCGACGCTGAATCCCGACATTACCGAAAGCCTGCCCTTGGGCTGCGT
AACCTACAGCCTGCGCTTCTTCCAAAACAAAATCACCGCCAAAGACTTCC
AATCTGCCATTTCCGCCGCCCGCAACGAAATCCAGCGTATCAGCAAAAAT
ATGAGGCGCGAAGGTTGGGATTTCGCCGTCGGCACATCGGGTTCGGCAAA
ATCCATCCGCGACGTGCTTGCCGCCGAAATGCCCCAAGAGGCGGACATTA
CCTACAAAGGCATGCGCGCCCTCGCCGAACGCATCATCGAAGCCGGTTCG
GTCAAAAAAGCCAAATTTGAAAACCTGAAACCGGAACGCATCGAAGTTTT
TGCCGGCGGACTTGCCGTGATGATGGCGGCGTTTGAGGAAATGAAACTCG
ACAGGATGACCGTAACCGAAGCCGCCCTGCGCGACGGCGTGTTTTACGAT
TTGATCGGGCGCGGTTTAAACGAAGATATGCGCGGACAAACGGTTGCCGA
GTTCCAACACCGCTACCACGTCAGCCTCAATCAGGCGAAACGCACCGCCG
AGACCGCGCAAACCTTTATGGACAGCCTCTGCCACGCTAAAAACGTTACA
CTTCAAGAGCTTGCCTTGTGGCAACAGTATCTCGGACGCGCCCCCGCGCT
GCACGAAATCGGTTTGGACATCGCCCACACCGGCTATCACAAGCATTCCG
CCTACATCCTCGAAAACGCCGATATGCCGGGTTTCTCACGCAAAGAACAG
ACCATACTTGCCCAACTGGTCATCGGTCATCGCGGCGATATGAAAAAAAT
GAGCGGCATCATCGGCACCAACGAAATGTTGTGGTATGCCGTTTTGTCCC
TGCGCCTTGCCGCACTGTTCTGCCGTTCGCGCCAAGACCTGTCTTTCCCG
AAAAATATGCAGTTGCGCACGGATACGGAAAGCTGCGGCTTCATCCTGCG
TATTGACAGGGAATGGCTGGAACGCCATCCCCTGATTGCCGACGCATTGG
AATATGAAAGCGTCCAATGGCAAAAAATCAATATGCCGTTCAAAGTCGAG
GCCGTCTGA
NMB1467 Protein sequence
MTTTPANVLASVDLGSNSFRLQICENNNGQLKVIDSFKOMVRFAAGLDEQ
KNLSAASQEQALDCLAKFGERLRGFRPEQVPAVATNTFRVAKNIADFLPK
AEAALGFPIEIIAGREEARLIYTGVIHTLPPGGGKMLVIDIGGGSTEFVI
GSTLNPDITESLPLGCVTYSLRFFQNKITAKDFQSAISAARNEIQRISKN
MRREGWDFAVGTSGSAKSIRDVLAAEMPQEADITYKGMRALAFRIIEAGS
VKKAKFENLKPERIEVFAGGLAVMMAAFEEMKLDRMTVTEAALRDGVPYD
LIGRGLNEDMRGQTVAEFQHRYHVSLNQAKRTAETAQTFMDSLCHAKNVT
VQELALWQQYLGRAAALHEIGLDIAHTGYHKHSAYILENADMPGFSRKEQ
TILAQLVIGHRGDMKKMSGIIGTNEMLWYAVLSLRLAALFCRSRQDLSFP
KNMQLRTDTESCGFILRIDREWLERHPLIADALEYESVQWQKINMPFKVE
AV
NMB2056 HemK
ATGAACGGTAAATACTACTACGGCACAGGCCGCCGCAAAAGTTCAGTGGC
TCGTGTATTCCTGATTAAAGGTACAGGTCAAATCATCGTAAACGGTCGTC
CCGTTGACGAATTCTTCGCACGGGAAACCAGCCGAATGGTTGTTCGCCAA
CCCTTGGTTCTGACTGAAAACGCCGAATCTTTCGACATCAAAGTCAATGT
TGTTGGCGGCGGCGAAACCGGCCAGTCCGGCGCAATCCGCCACGGCATTA
CCCGTGCCCTGATCGACTTCGATGCCGCGTTGAAACCCGCCTTGTCTCAA
GCTGGTTTTGTTACCCGCGATGCCCGCGAAGTCGAACGTAAAAAACCGGG
TCTGCGCAAAGCACGCCGTGCAAAACAATTCTCCAAACGTTAA
NMB2056 Protein sequence
MNGKYYYGTGRRKSSVARVFLIKGTGQIIVNGRPVDEFFARETSRMVVRQ
PLVLTENAESFDIKVNVVGGGETGQSGAIRHGITRALIDFDAALKPALSQ
AGFVTRDAREVERKKPGLRKARRAKQFSKR
NMB0808 DNA sequence
ATGTCCGCCCTCCTCCCCATCATCAACCGCCTGATTCTGCAAAGCCCGGA
CAGCCGCTCGGAACTTGCCGCCTTTGCAGGCAAAACACTGACCCTGAACA
TTGCCGGGCTGAAACTGGCGGGACGCATCACGGAAGACGGTTTGCTCTCG
GCGGGAAACGGCTTTGCAGACACCGAAATTACCTTCCGCAACAGCGCGGT
ACAGAAAATCCTCCAAGGAGGCGAACCCGGGGCGGGCGACATCGGGCTCG
AAGGCGACCTCATCCTCGGCATCGCGGTACTGTCCCTGCTCGGCAGCCTG
CGTTCCCGCGCATCGGACGAATTGGCACGGATTTTCGGCACGCAGGCAGA
CATCGGCAGCCGTGCCGCCGACATCGGACACGGCATCAAACAAATCGGCA
GGAACATCGCCGAACAAATCGGCGGATTTTCCCGCGAATCCGAGTCCGCA
AACATCGGCAACGAAGCCCTTGCCGACTGCCTCGACGAAATAAGCAGACT
GCGCGACGGCGTGGAACGCCTCAACGAACGCCTCGACCGGCTCGAACGCG
ACATTTGGATAGACTAA
NMB0808 Protein sequence
MSALLPIINRLILQSPDSRSELAAFAGKTLTLNIAGLKLAGRITEDGLLS
AGNGFADTEITFRNSAVQKILQGGEPGAGDTGLEGDLILGIAVLSLLGSL
RSPASDELARIFGTQADIGSRAADIGHGIKQIGRNIAEQIGGFSRESESA
NIGNEALADCLDEISRLRDGVERLNERLDRLERDIWID
NMB0774 upp DNA sequence
ATGAACGTTAATGTTATCAACCATCCGCTCGTCCGCCACAAATTAACCCT
GATGAGGGAGGCGGATTGCAGCACCTACAAATTCCGGACGCTTGCCACCG
AGCTGGCGCGCCTGATGGCATACGAGGCAAGCCGTGATTTTGAAATCGAA
AAATACCTTATCGACGGATGGTGCGGTCAGATTGAAGGCGACCGCATCAA
GGGCAAAACATTGACCGTCGTTCCCATACTGCGTGCAGGTTTGGGTATGC
TTGACGGTGTGCTCGACCTGATTCCGACTGCCAAAATCAGTGTAGTCGGA
CTGCAGCGCGACGAAGAAACGCTGAAGCCTATTTCCTATTTTGAGAAATT
TGTGGACAGTATGGACGAACGTCCGGCTTTGATTATCGATCCTATGCTGG
CGACAGGCGGTTCGATGGTTGCCACCATCGACCTTTTGAAAGCCAAGGGC
TGCAAAAATATCAAGGCACTGGTGCTGGTTGCCGCGCCCGAGGGTGTGAA
GGCGGTCAACGACGCGCACCCTGACGTTACGATTTACACCGCCGCGCTCG
ACAGCCACTTGAACGAGAACGGCTACATCATCCCCGGCTTGGGCGATGCG
GGCGACAAGATTTTCGGCACGCGCTAA
NMB0774 Protein sequence
MNVNVINHPLVRHKLTLMREADCSTYKFRTLATELARLMAYEASRDFEIE
KYLIDGWCGQIEGDRIKGKTLTVVPILRAGLGMLDGVLDLIPTAKISVVG
LQRDEETLKPISYFEKFVDSMDERPALIIDPMLATGGSMVATIDLLKAKG
CKNIKALVLVAAPEGVKAVNDAHPDVTIYTAALDSHLNENGYIIPGLGDA
GDKIFGTR
NMA0078 putative integral membrance protein DNA
sequence
TTGGCGTTTACTTTAATGCGTCGCGCCATGATACGTAAAATGCCCTATAC
GGAAGATATGCGCCCAGGCGATACCGCTAATCCTTATGGTGCGTCCAAAG
CGATGGTGGAACGGATGTTAACCGACATCCAAAAAGCCGATCCGCGCTGG
AGCATGATTTTGTTGCGTTATTTCAATCCGATTGGCGCGCATGAAAGCGG
CTTGATTGGCGAGCAGCCAAACGGCATCCCGAATAATTTGTTGCCTTATA
TCTGCCAAGTGGCGGCAGGCAAACTGCCGCAATTGGCGGTATTTGGCGAT
GACTACCCTACCCCCGACGGCACGGGGATGCGTGACTATATTCATGTGAT
GGATTTGGCAGAAGGCCATGTCGCGGCTATGCAGGCAAAAAGTAATGTAG
CAGGCACGCATTTGCTGAACTTAGGCTCCGGCCGCGCTTCTTCGGTGTTG
GAAATCATCCGCGCATTTGAAGCAGCTTCGGGTTTGACGATTCCGTATGA
AGTCAAACCGCGCCGTGCCGGTGATTTGGCGTGCTTCTATGCCGACCCTT
CCTATACAAAGGCGCAAATCGGCTGGCAAACCCAGCGTGATTTAACCCAA
ATGATGGAAGACTCATGGCGCTGGGTGAGTAATAATCCGAATGGCTACGA
CGATTAA
NMA0078 Protein sequence
MAFTLMRRAMIRKMPYTEDMRPGDTANPYGASKAMVERMLTDIQKADPRW
SMILLRYFNPIGAHESGLIGEQPNGIPNNLLPYICQVAAGKLPQLAVFGD
DYPTPDGTGMRDYIHVMDLAEGHVAAMQAKSNVAGTHLLNLGSGPASSVL
EIIRAFEAASGLTIPYEVKPRRAGDLACFYADPSYTKAQIGWQTQRDLTQ
MMEDSWRWVSNNPNGYDD
NMB0337 Branched-chain amino acid aminotransferase
DNA sequence
ATGAGCAGACCCGTACCCGCCGTATTCGGCAGCGTTTTTCACAGTCAAAT
GCCCGTCCTCGCCTACCGCGAAGGCAAATGGCAGCCGACCGAATGGCAAT
CTTCCCAAGACCTCTCCCTCGCACCGGGCGCGCACGCCCTGCACTACGGC
AGCGAATGTTTCGAGGGACTGAAAGCCTTCCGTCAGGCAGACGGCAAAAT
CGTGCTGTTCCGTCCGACTGCCAATATCGCGCGTATGCGGCAAAGTGCGG
ACATTTTGCACCTGCCGCGCCCCGAAACCGAAGCTTATCTTGACGCGCTA
ATCAAATTGGTCAAACGTGCCGCCGATGAAATTCCCGATGCGCCTGCCGC
CCTGTACCTGCGTCCGACCTTAATCGGTACCGATCCCGTTATCGGCAAGG
CCGGTTCTCCTTCCGAAACCGCCCTGCTGTATATTTTGGCTTCCCCCGTC
GGCGACTATTTCAAAGTCGGATCGCCCGTCAAAATTTTGGTGGAAACCGA
ACACATCCGCTGCGCCCCGCATATGGGCCGCGTCAAATGCGGCGGCAACT
ACGCTTCCGCCATGCACTGGGTGCTGAAGGCGAAAGCCGAATATGGCGCA
AATCAAGTCCTGTTCTGCCCGAACGGCGACGTGCAGGAAACCGGCGCGTC
CAACTTTATCCTGATTAACGGCGATGAAATCATTACCAAACCGCTGACCG
ACGAGTTTTTGCACGGCGTAACCCGCGATTCCGTACTGACGGTTGCCAAA
GATTTGGGCTATACCGTCAGCGAACGCAATTTCACGGTTGACGAACTCAA
AGCTGCGGTGGAAAACGGTGCGGAAGCCATTTTGACCGGTACGGCAGCCG
TCATCTCGCCCGTTACTTCCTTCGTCATCGGCGGCAAAGAAATCGAAGTG
AAAAGCCAAGAACGCGGCTATGCCATCCGTAAGGCGATTACCGACATCCA
GTATGGTTTGGCGGAAGACAAATACGGCTGGCTGGTTGAAGTGTGCTGA
NMB0337 Protein sequence
MSRPVPAVFGSVFHSQMPVLAYREGKWQPTEWQSSQDLSLAPGAHALHYG
SECFEGLKAFRQADGKIVLFRPTANIARMRQSADILHLPRPETEAYLDAL
IKLVKRPADEIPDAPAALYLRPTLIGTDPVIGKAGSPSETALLYILASPV
GDYFKVGSPVKILVETEHIRCAPHMGRVKCGGNYASAMHWVLKAKAEYGA
NQVLFCPNGDVQETGASNFILINGDEIITKPLTDEFLHGVTRDSVLTVAK
DLGYTVSERNFTVDELKAAVENGAEAILTGTAAVISPVTSFVIGGKEIEV
KSQERGYAIRKAITDIQYGLAEDKYGWLVEVC
NMB0191 ParA family protein DNA sequence
ATGAGTGCGAACATCCTTGCCATCGCCAATCAGAAGGGCGGTGTGGGCAA
AACGACGACGACGGTAAATTTGGCGGCTTCGCTGGCATCGCGCGGCAAAC
GCGTGCTGGTGGTCGATTTGGATCCGCAGGGCAATGCGACGACGGGCAGC
GGCATCGACAAGGCGGGTTTGCAGTCCGGCGTTTATCAGGTCTTATTGGG
CGATGCGGACGTGCAGTCGGCGGCGGTACGCAGCAAAGAGGGCGGATACG
CTGTGTTGGGTGCGAACCGCGCGCTGGCCGGCGCGGAAATCGAACTGGTG
CAGGAAATCGCCCGGGAAGTGCGTTTGAAAAACGCGCTCAAGGCAGTGGA
AGAAGATTACGACTTTATCCTGATCGACTGCCCGCCTTCGCTGACGCTGT
TGACGCTTAACGGGCTGGTGGCGGCGGGCGGCGTGATTGTGCCGATGTTG
TGCGAATATTACGCGCTGGAAGGGATTTCCGATTTGATTGCGACCGTGCG
CAAAATCCGTCAGGCGGTCAATCCCGATTTGGACATCACGGGCATCGTGC
GCACGATGTACGACAGCCGCAGCAGGCTGGTTGCCGAAGTCAGCGAACAG
TTGCGCAGCCATTTCGGGGATTTGCTTTTTGAAACCGTCATCCCGCGCAA
TATCCGCCTTGCGGAAGCGCCGAGCCACGGTATGCCGGTGATGGCTTACG
ACGCGCAGGCAAAGGGTACCAAGGCGTATCTTGCCTTGGCGGACGAGCTG
GCGGCGAGGGTGTCGGGGAAATAG
NMB0191 Protein sequence
MSANILAIANQKGGVGKTTTTVNLAASLASRGKRVLVVDLDPQGNATTGS
GIDKAGLQSGVYQVLLGDADVQSAAVRSKEGGYAVLGANRALAGAEIELV
QEIAREVRLKNALKAVEEDYDFILIDCPPSLTLLTLNGLVAAGGVIVPML
CEYYALEGISDLIATVRKIRQAVNPDLDITGIVRTMYDSRSRLVAEVSEQ
LRSHFGDLLFETVIPRNIRLAEAPSHGMPVMAYDAQAKGTKAYLALADEL
AARVSGK
NMB1710 Glutamate dehydrogenase(gdhA) DNA sequence
ATGACTGACCTGAACACCCTGTTTGCCAACCTCAAACAACGCAATCCCAA
TCAGGAGCCGTTCCATCAGGCGGTTGAAGAAGTCTTCATGAGTCTCGATC
CGTTTTTGGCAAAAAATCCGAAATACACCCAGCAAAGCCTGCTGGAACGC
ATCGTCGAACCCGAACGCGTCGTGATCTTCCGCGTAACCTGGCAGGACGA
TAAAGGGCAAGTCCAAGTCAACCGGGGCTACCGCGTGCAAATGAGTTCCG
CCATCGGTCCTTACAAAGGCGGCCTGCGCTTCCATCCGACCGTCGATTTG
GGCGTATTGAAATTCCTCGCTTTTGAACAAGTGTTCAAAAACGCCTTGAC
CACCCTGCCTATGGGCGGCGGCAAAGGCGGTTCCGACTTCGACCCCAAAG
GCAAATCCGATGCCGAAGTAATGCGCTTCTGCCAAGCCTTTATGACCGAA
CTCTACCGCCACATCGGCGCGGACACCGATGTTCCGGCCGGCGACATCGG
CGTAGGCGGGCGCGAAATCGGCTACCTGTTCGGACAATACAAAAAAATCC
GCAACGAGTTTTCTTCCGTCCTGACCGGCAAAGGTTTGGAATGGGGCGGC
AGCCTCATCCGTCCCGAAGCGACCGGCTACGGCTGCGTCTATTTCGCCCA
AGCGATGCTGCAAACCCGCAACGATAGTTTTGAAGGCAAACGCGTCCTGA
TTTCCGGCTCCGGCAATGTGGCGCAATACGCCGCCGAAAAAGCCATCCAA
CTGGGTGCGAAAGTACTGACCGTTTCCGACTCCAACGGCTTCGTCCTCTT
CCCCGACAGCGGTATGACCGAAGCGCAACTCGCCGCCTTGATCGAATTGA
AAGAAGTCCGCCGCGAACGCGTTGCCACCTACGCCAAAGAGCAAGGTCTG
CAATACTTTGAAAAACAAAAACCGTGGGGCGTCGCCGCCGAAATCGCCCT
GCCCTGCGCGACCCAGAACGAATTGGACGAAGAAGCCGCCAAAACCCTGT
TGGCAAACGGCTGCTACGTCGTTGCCGAAGGTGCGAATATGCCGTCGACT
TTGGGCGCGGTCGAGCAATTTATCAAAGCCGGCATCCTCTACGCCCCGGG
AAAAGCCTCCAATGCCGGCGGCGTGGCAACTTCAGGTTTGGAAATGAGCC
AAAACGCCATCCGCCTGTCTTGGACTCGTGAAGAAGTCGACCAACGCCTG
TTCGGCATCATGCAAAGCATCCACGAATCCTGTCTGAAATACGGCAAAGT
CGGCGACACAGTAAACTACGTCAATGGTGCGAACATTGCCGGTTTCGTCA
AAGTTGCCGATGCGATGCTGGCGCAAGGCTTCTAA
NMB1710 Protein sequence
MTDLNTLFANLKQRNPNQEPFHQAVEEVFMSLDPFLATNPKYTQQSLLER
IVEPERVVMFRVTWQDDKGQVQVNRGYRVQMSSAIGPYKCGLRFHPTVDL
GVLKFLAFEQVFKNALTTLPMGGGKGGSDFDPKGKSDAEVMRFCQAFMTE
LYRHIGADTDVPAGDIGVGGREIGYLFGQYKKIRNEFSSVLTGKGLEWGG
SLIRPEATGYGCVYFAQAMLQTRNDSFEGKRVLISGSGNVAQYAAEKAIQ
LGAKVLTVSDSNGFVLFPDSGMTEAQLAALIELKEVRRERVATYAKEQGL
QYFEKQKPWGVAAEIALPCATQNELDEEAAKTLLANGCYVVAEGANMPST
LGAVEQFIKAGILYAPGKASNAGGVATSGLEMSQNAIRLSWTREEVDQRL
FGIMQSIHESCLKYGKVGDTVNYVNGANIAGFVKVADAMLAQGF
NMB0062 Glucose-1-phosphate thymidylytransferase
(rfbA-1) DNA sequence
ATGAAACGCATCATACTGGCAGGCGGCAGCGGCACGCGCCTCTACCCCAT
CACGCGCGGCGTATCCAAACAGCTCCTGCCCGTGTACGACAAACCGATGA
TTTATTACCCCTTGTCGGTTTTGATGCTGGCGGGAATCCGCGATATTTTG
GTGATTACCGCGCCTGAAGACAACGCCTCTTTCAAACGCCTGCTTGGCGA
CGGCAGCGATTTCGGCATTTCCATCAGTTATGCCGTGCAACCCAGTCCGG
ACGGCTTGGCACAGGCATTTATCATCGGCGAAGAATTTATCGGCAACGAC
AATGTTTGCTTGGTTTTGGGCGACAATATTTTTTACGGTCAGTCGTTTAC
GCAAACATTGAAACAGGCGGCAGCGCAAACGCACGGCGCAACCGTGTTTG
CTTATCAGCTCAAAAACCCCGAACGTTTCGGCGTGGTTGAATTTAACGAA
AACTTCCGCGCCGTTTCCATCGAAGAAAAACCGCAACGGCCCAAATCCGA
TTGGGCGGTAACCGGCTTGTATTTCTACGACAACCGCGCCGTCGAGTTCG
CCAAACAGCTCAAACCGTCCGCACGCGGCGAATTGGAAATTACCGACCTC
AACCGGATGTATTTGGAAGACGGCTCGCTCTCCGTTCAAATATTGGGACG
CGGTTTCGCGTGGCTGGACACCGGCACCCACGAGAGCCTGCACGAAGCCG
CTTCATTCGTCGAAACCGTGCAAAATATCCAAAACCTGCACATCGCCTGC
CTCGAAGAAATCGCTTGGCGCAACGGTTGGCTTTCCGATGAAAAACTGGA
AGAATTGGCGCGCCCGATGGCGAAAAACCAATACGGCCAATATTTGCTGC
GCCTGTTGAAAAAATAA
NMB0062 Protein sequence
MKGIILAGGSGTRLYPITRGVSKQLLPVYDKPMIYYPLSVLMLAGIRDIL
VITAPEDNASFKRLLGDGSDFGISISYAVQPSPDGLAQAFIIGEEFIGND
NVCLVLGDNIFYGQSFTQTLKQAAAQTHGATVFAYQVKNPERFGVVEFNE
NFRAVSIEEKPQRPKSDWAVTGLYFYDNRAVEFAKQLKPSARGELEITDL
NRMYLEDGSLSVQILGRGFAWLDTGTHESLHEAASFVQTVQNIQNLHIAC
LEEIAWRNGWLSDEKLEELARPMAKNQYGQYLLRLLKK
NMB1583 Imidazoleglycerol-phosphate dehydratase
(hisB) DNA sequence
ATGAATTTGACTAAAACACAACGCCAACTGCACAACTTTCTGACCCTCGC
CCAAGAAGCAGGTTCGCTGTCCAAGCTCGCCAAACTCTGCGGCTACCGTA
CCCCCGTCGCACTCTACAAACTCAAACAACGCCTTGAAAAGCAGGCAGAA
GACCCAGATGCACGCGGCATCCGTCCCAGCCTGATGGCAAAACTCGAAAA
ACACACCGGCAAACCCAAAGGCTGGCTCGACAGAAAACACCGCGAACGCA
CTGTCCCCGAAACCGCCGCAGAAAGCACCGGAACTGCCGAAACCCAAATT
GCCGAAACCGCATCTGCTGCCGGCTGCCGCAGCGTTACCGTCAACCGCAA
TACCTGCGAAACCCAAATCACCGTCTCCATCAACCTCGACGGCAGCGGCA
AAAGCAGGCTGGATACCGGCGTACCCTTCCTCGAACACATGATCGATCAA
ATCGCCCGCCACGGCATGATTGACATCGACATCAGCTGCAAAGGCGACCT
GCACATCGACGACCACCACACCGCCGAAGACATCGGCATCACACTCGGAC
AAGCAATCCGGCAGGCACTCGGCGACAAAAAAGGCATCCGCCGTTACGGA
CATTCCTACGTCCCGCTCGACGAAGCCCTCAGCCGCGTCGTCATCGACCT
TTCCGGCCGCCCCGGACTCGTGTACAACATCGAATTTACCCGCGCACTAA
TCGGACGTTTCGATGTCGATTTGTTTGAAGAATTTTTCCACGGCATCGTC
AACCACAGTATGATCACCCTGCACATCGACAACCTCAGCGGCAAAAACGC
CCACCATCAGGCGGAAACCGTATTCAAAGCCTTCGGGCGCGCCCTGCGTA
TGGCAGTCGAACACGACCCGCGCATGGCAGGACAGACCCCCTCGACCAAA
GGCACGGTGACCGCATAA
NMB1583 Protein sequence
MNLTKTQRQLHNFLTLAQEAGSLSKLAKLCGYRTPVALYKLKQRLEKQAE
DPDARGIRPSLMAKLEKHTGKPKGWLDRKHRERTVPETAAESTGTABTQI
AETASAAGCRSVTVNRNTCETQITVSINLDGSGKSRLDTGVPFLEHMIDQ
IARHGMIDIDISCKGDLHIDDHHTAEDIGITLGQAIRQALGDKKGIRRYG
HSYVPLDEALSRVVIDLSGRPGLVYNIEFTRALIGRFDVDLFEEFFHGIV
NHSMMTLHIDNLSGKNAHHQAETVFKAFGRALRMAVEHDPRMAGQTPSTK
GTLTA

Schedule of SEQ ID Nos

| | |
|---|---|
| SEQ ID No | Sequence |

| | |
|---|---|
| 1 | NMB0341 DNA |
| 2 | NMB0341 Protein |
| 3 | NMB0338 DNA |
| 4 | NMB0338 Protein |
| 5 | NMB1345 DNA |
| 6 | NMB1345 Protein |
| 7 | NMB0738 DNA |
| 8 | NMB0738 Protein |
| 9 | NMB0792 DNA |
| 10 | NMB0792 Protein |
| 11 | NMB0279 DNA |
| 12 | NMB0279 Protein |
| 13 | NMB2050 DNA |
| 14 | NMB2050 Protein |
| 15 | NMB1335 DNA |
| 16 | NMB1335 Protein |
| 17 | NMB2035 DNA |
| 18 | NMB2035 Protein |
| 19 | NMB1351 DNA |
| 20 | NMB1351 Protein |
| 21 | NMB1574 DNA |
| 22 | NMB1574 Protein |
| 23 | NMB1298 DNA |
| 24 | NMB1298 Protein |
| 25 | NMB1856 DNA |
| 26 | NMB1856 Protein |
| 27 | NMB0119 DNA |
| 28 | NMB0119 Protein |
| 29 | NMB1705 DNA |
| 30 | NMB1705 Protein |
| 31 | NMB2065 DNA |
| 32 | NMB2065 Protein |
| 33 | NMB0339 DNA |
| 34 | NMB0339 Protein |
| 35 | NMB0401 DNA |
| 36 | NMB0401 Protein |
| 37 | NMB1467 DNA |
| 38 | NMB1467 Protein |
| 39 | NMB2056 DNA |
| 40 | NMB2056 Protein |
| 41 | NMB0808 DNA |
| 42 | NMB0808 Protein |
| 43 | NMB0774 DNA |
| 44 | NMB0774 Protein |
| 45 | NMA0078 DNA |
| 46 | NMA0078 Protein |
| 47 | NMB0337 DNA |
| 48 | NMB0337 Protein |
| 49 | NMB0191 DNA |
| 50 | NMB0191 Protein |
| 51 | NMB1710 DNA |
| 52 | NMB1710 Protein |
| 53 | NMB0062 DNA |
| 54 | NMB0O62 Protein |
| 55 | NMB1583 DNA |
| 56 | NMB1583 Protein |

1. A method for identifying a polypeptide of a microorganism which polypeptide is associated with an immune response in an animal which has been subjected to the microorganism, the method comprising the steps of
(1) providing a plurality of different mutants of the microorganism;
(2) contacting the plurality of mutant microorganisms with antibodies from an animal which has raised an immune response to the microorganism or a part thereof, under conditions whereby if the antibodies bind to the mutant microorganism the mutant microorganism is killed;
(3) selecting surviving mutant microorganisms from step (2);
(4) identifying the gene containing the mutation in any surviving mutant microorganism; and
(5) identifying the polypeptide encoded by the gene.

2. A method according to claim 1 wherein the microorganism is a pathogenic microorganism.

3. A method according to claim 1 wherein the animal subjected to the microorganism is a host of the microorganism.

4. A method according to claim 3 wherein the animal subjected to the microorganism is a human who is or has been infected with the microorganism.

5. A method according to claim 1 wherein the microorganism is a bacterium.

6. A method according to claim 5 wherein the bacterium is Neisseria menigitidis.

7. A method according to claim 1 wherein the mutant microorganisms have insertional mutations.

8. A method according to claim 1 wherein any surviving mutant selected in step (3) is backcrossed into a parental strain of the microorganism and it is determined whether the resulting cross is resistant to killing under conditions as set out in step (2).

9. A method according claim 1 wherein in step (2) complement mediates killing of the microorganisms to which the antibodies are bound.

10. A method of identifying a gene encoding a polypeptide of a microorganism which polypeptide is associated with an immune response in an animal which has been subjected to the microorganism, the method comprising carrying out steps (1) to (4) as defined in claim 1.

11. A method of selecting a microorganism mutated in a gene encoding a polypeptide which polypeptide is associated with an immune response in an animal which has been subjected to the microorganism, the method comprising carrying out steps (1) to (3) as defined in claim 1.

12. A method for making an antigen the method comprising carrying out the method according to claim 1 and synthesising the polypeptide identified in step (5) or an antigenic fragment or variant thereof, or fusion of such polypeptide or fragment or variant.

13. A method according to claim 12 wherein the variant is a homologous polypeptide from a related microorganism.

14. A method for making a vaccine for combating a microorganism the method comprising making an antigen according to the method of claim 12 or polynucleotide encoding said antigen and combining the antigen or polynucleotide, with a suitable carrier.

15. A method according to claim 14 wherein the antigen or polynucleotide is combined with an adjuvant.

16. An antigen obtainable according to the method of claim 12 or a polynucleotide encoding said antigen.

17. A vaccine obtainable by the method of claim 14.

18. An antigen or polynucleotide according to claim 16 for use in a vaccine.

19. A method of vaccinating an individual against a microorganism, the method comprising administering an antigen or polynucleotide according to claim 16, to the individual.

20. (canceled)

21. A method for making a polynucleotide the method comprising carrying out the steps of claim 10 and isolating or synthesising the identified gene or a variant or fragment thereof or a fusion of such gene or variant or fragment.

22. A polynucleotide obtainable by the method of claim 21.

23. A mutant microorganism obtainable by the method of claim 11.

24. (canceled)

25. A polypeptide comprising the amino acid sequence selected from any one of SEQ ID Nos 4, 2, 6, 8, 10, 12, 14, 16, 18, 20, 22, 24, 26, 28, 30, 32, 34, 36, 38, 40, 42, 44, 46, 48, 50, 52, 54, 56; or a or variant thereof or a fusion of such a fragment or variant.

26. A polynucleotide encoding a polypeptide according to claim 25.

27. A polypeptide according to claim 25 or a polynucleotide encoding the polypeptide, each of which is for use in medicine.

28. A polypeptide according to claim 25 or a polynucleotide encoding the polypeptide, each of which is for use in a vaccine.

29. A method for making a polypeptide according to claim 25, the method comprising expressing a polynucleotide encoding the polypeptide in a host cell and isolating said polypeptide.

30. A method for making a polypeptide according to claim 26 comprising chemically synthesising said polypeptide.

31. A method of vaccinating an individual against Neisseria meningitidis, the method comprising administering to the individual a polypeptide according to claim 25 or a polynucleotide encoding the polypeptide.

32. (canceled)

33. (canceled)
